SEQUENCE LISTING (45 sequences)

SEQ ID NO: 1 - DNA, 23 bp, synthetic construct
  source 1..23: mol_type = other DNA
gcaagacagaagcttcacgactc

SEQ ID NO: 2 - DNA, 20 bp, synthetic construct
  source 1..20: mol_type = other DNA
gcaatatggtggaaaataac

SEQ ID NO: 3 - DNA, 20 bp, synthetic construct
  source 1..20: mol_type = other DNA
ccagatgactacctatcctc

SEQ ID NO: 4 - DNA, 20 bp, synthetic construct
  source 1..20: mol_type = other DNA
gagctgcagtggagtaggcg

SEQ ID NO: 5 - DNA, 20 bp, synthetic construct
  source 1..20: mol_type = other DNA
ggttaaacccagcttgacca

SEQ ID NO: 6 - DNA, 20 bp, synthetic construct
  source 1..20: mol_type = other DNA
ggaggcagagacagttggag

SEQ ID NO: 7 - DNA, 24 bp, synthetic construct
  source 1..24: mol_type = other DNA
ccaaagtcgctctgagttgttatc

SEQ ID NO: 8 - DNA, 20 bp, synthetic construct
  source 1..20: mol_type = other DNA
gagcgggagaaatggatatg

SEQ ID NO: 9 - DNA, 19 bp, synthetic construct
  source 1..19: mol_type = other DNA
ccaagagggtcaagttgga

SEQ ID NO: 10 - DNA, 19 bp, synthetic construct
  source 1..19: mol_type = other DNA
ccaagagggtcaagttgga

SEQ ID NO: 11 - DNA, 25 bp, synthetic construct
  source 1..25: mol_type = other DNA
gtcatggcaatgccaaggtcgctag

SEQ ID NO: 12 - DNA, 21 bp, synthetic construct
  source 1..21: mol_type = other DNA
tgttgggacaaacgagcaatc

SEQ ID NO: 13 - DNA, 22 bp, synthetic construct
  source 1..22: mol_type = other DNA
ggtggctggaccaatgtaaata

SEQ ID NO: 14 - DNA, 20 bp, synthetic construct
  source 1..20: mol_type = other DNA
tggatccaggataacggagg

SEQ ID NO: 15 - DNA, 20 bp, synthetic construct
  source 1..20: mol_type = other DNA
tgttgacttcacttgtggcc

SEQ ID NO: 16 - DNA, 24 bp, synthetic construct
  source 1..24: mol_type = other DNA
ctaggccacagaattgaaagatct

SEQ ID NO: 17 - DNA, 25 bp, synthetic construct
  source 1..25: mol_type = other DNA
gtaggtggaaattctagcatcatcc

SEQ ID NO: 18 - DNA, 21 bp, synthetic construct
  source 1..21: mol_type = other DNA
agagagcctcactcccattcc

SEQ ID NO: 19 - DNA, 20 bp, synthetic construct
  source 1..20: mol_type = other DNA
cttcaaacttcatcagatcg

SEQ ID NO: 20 - DNA, 19 bp, synthetic construct
  source 1..19: mol_type = other DNA
tgtctttccccagcacagt

SEQ ID NO: 21 - DNA, 23 bp, synthetic construct
  source 1..23: mol_type = other DNA
ctgcatagtacgctataccctgt

SEQ ID NO: 22 - DNA, 20 bp, synthetic construct
  source 1..20: mol_type = other DNA
gcaggtcgagggacctaata

SEQ ID NO: 23 - DNA, 17 bp, synthetic construct
  source 1..17: mol_type = other DNA
ctggcttctgaggaccg

SEQ ID NO: 24 - DNA, 18 bp, synthetic construct
  source 1..18: mol_type = other DNA
caggacaacgcccacaca

SEQ ID NO: 25 - DNA, 18 bp, synthetic construct
  source 1..18: mol_type = other DNA
agggcgaggagctgttca

SEQ ID NO: 26 - DNA, 19 bp, synthetic construct
  source 1..19: mol_type = other DNA
tgaagtcgatgcccttcag

SEQ ID NO: 27 - DNA, 20 bp, synthetic construct
  source 1..20: mol_type = other DNA
agtgcgtgcagaaggagttt

SEQ ID NO: 28 - DNA, 20 bp, synthetic construct
  source 1..20: mol_type = other DNA
cacaaattctcggcagtcaa

SEQ ID NO: 29 - DNA, 23 bp, synthetic construct
  source 1..23: mol_type = other DNA
acataatacactaaatggagccc

SEQ ID NO: 30 - DNA, 21 bp, synthetic construct
  source 1..21: mol_type = other DNA
atacagtcctggatgatgatg

SEQ ID NO: 31 - DNA, 21 bp, synthetic construct
  source 1..21: mol_type = other DNA
aaagtcgctctgagttgttat

SEQ ID NO: 32 - DNA, 21 bp, synthetic construct
  source 1..21: mol_type = other DNA
ggagcgggagaaatggatatg

SEQ ID NO: 33 - DNA, 21 bp, synthetic construct
  source 1..21: mol_type = other DNA
gcgaagagtttgtcctcaacc

SEQ ID NO: 34 - DNA, 31 bp, synthetic construct
  source 1..31: mol_type = other DNA
gggtctagactctcagccggctccctcaggg

SEQ ID NO: 35 - DNA, 30 bp, synthetic construct
  source 1..30: mol_type = other DNA
gcgaagcttctcacagagcctgtccatcac

SEQ ID NO: 36 - DNA, 30 bp, synthetic construct
  source 1..30: mol_type = other DNA
gcgaagcttgtggagtctgggggaggctta

SEQ ID NO: 37 - DNA, 30 bp, synthetic construct
  source 1..30: mol_type = other DNA
gcgaagcttargcctgggrcttcagtgaag

SEQ ID NO: 38 - DNA, 21 bp, synthetic construct
  source 1..21: mol_type = other DNA
atgcccctcaacgtgaacttc

SEQ ID NO: 39 - DNA, 21 bp, synthetic construct
  source 1..21: mol_type = other DNA
gtcgcagatgaaatagggctg

SEQ ID NO: 40 - DNA, 19 bp, synthetic construct
  source 1..19: mol_type = other DNA
tgctccatgaggagacacc

SEQ ID NO: 41 - DNA, 20 bp, synthetic construct
  source 1..20: mol_type = other DNA
ctctgaccttttgccaggag

SEQ ID NO: 42 - DNA, 22 bp, synthetic construct
  source 1..22: mol_type = other DNA
gcttcgtgttcaccaaggagga

SEQ ID NO: 43 - DNA, 22 bp, synthetic construct
  source 1..22: mol_type = other DNA
gtcctagaccagtgttctgagc

SEQ ID NO: 44 - DNA, 13 bp, synthetic construct
  source 1..13: mol_type = other DNA
ataacttcgtata

SEQ ID NO: 45 - DNA, 13 bp, synthetic construct
  source 1..13: mol_type = other DNA
gaagttcctattc

(19) United States
(12) Patent Application Publication
(10) Pub. No.: US 2026/0026479 A1
(43) Pub. Date: Jan. 29, 2026

(54) GENETICALLY ENGINEERED MICE MODELS FOR MULTIPLE MYELOMA

(71) Applicant: FUNDACIÓN PARA LA INVESTIGACIÓN MÉDICA APLICADA, Pamplona (ES)

(72) Inventors: José Angel MARTÍNEZ CLIMENT, Pamplona (ES); MARTA LARRÁYOZ ILUNDÁIN, Pamplona (ES); María José GARCÍA BARCHINO, Pamplona (ES); JON CELAY LEOZ, Pamplona (ES)

(21) Appl. No.: 19/099,247

(22) PCT Filed: Jul. 28, 2023
(86) PCT No.: PCT/EP2023/071025

§ 371 Date: Jan. 28, 2025

(30) Foreign Application Priority Data
Jul. 29, 2022 (EP) 22382736.1

(51) Int. Cl. A01K 67/0278; C07K 14/47; C12N 9/10; C12N 9/12

(52) U.S. Cl. CPC A01K 67/0278; C07K 14/4747; C12N 9/1007; C12N 9/12; C12N 9/1241; A01K 2217/075; A01K 2227/105; A01K 2267/0331; A01K 2267/0393; C12Y 207/1101

(57) ABSTRACT

The invention relates to genetically engineered mouse models for multiple myeloma (MM) and their uses thereof for the development of multiple myeloma models as well as for the screening of compounds suitable for the treatment of multiple myeloma.

### FIELD OF THE INVENTION

The present invention is comprised within the field of biotechnology. It specifically relates to genetically engineered mouse models for multiple myeloma (MM) and their uses thereof.

### BACKGROUND OF INVENTION

Multiple myeloma (MM) is a disease of clonal malignant plasma cells (PCs) that accumulate in the bone marrow (BM) and secrete a monoclonal immunoglobulin (Ig), which induces progressive multi-organ damage. The disease represents 2% of all cancers and occurs predominantly in the elderly population, although a pre-malignant condition termed monoclonal gammopathy of undetermined significance (MGUS) is present years before clinically evident disease. Transition of MGUS into MM often occurs via an intermediate stage termed asymptomatic (smoldering) MM (SMM), with a risk of progression to symptomatic MM of 10% per year. Genetic heterogeneity is a hallmark of MM, which is thought to be initiated by chromosomal translocations targeting CCND1 (encoding cyclin D1), MMSET or MAF oncogenes occurring at germinal center B lymphocytes or in B lymphoid progenitors. Then, these abnormal B cells acquire secondary genetic alterations in NF-κB, RAS and apoptotic pathways while differentiating into class-switched plasmablasts, which leave the lymphoid follicles to reach the BM. There, they progressively expand as pre-malignant PCs in close interaction with the immune cell microenvironment, eventually originating malignant tumors. Additional genetic changes in MYC and P53 genes are later acquired in clinically aggressive, relapsed and refractory MM. While this complex stepwise path of MM development is widely accepted, clear experimental evidences demonstrating this transformation process are lacking.

Standard-of-care induction therapy in MM consists in bortezomib, lenalidomide, and dexamethasone (VRD regimen), which can also include a monoclonal antibody against the surface cell receptor CD38. Therapy is then followed by autologous stem cell transplantation in eligible patients, and then by maintenance with lenalidomide or bortezomib on the basis of genetic risk factors. At relapse, varied combinations of clinically approved carfilzomib, ixazomib, pomalidomide, panobinostat and/or immunotherapy drugs including daratumumab and elotuzumab can be used. Despite these effective therapies lead to deep remissions and durable clinical responses that extend life for most MM patients, the disease invariably relapses and remains virtually incurable. Therefore, the goal of current clinical trials is to identify novel and more effective immune-based drug combinations with the intention of curing the disease.

Undoubtedly, the design of the next generation clinical trials will enormously benefit from pre-clinical studies in experimental models of MM. However, these studies are seriously hampered by two principal factors: 1) human MGUS and MM primary cells cannot be properly grown in vitro nor in immunocompetent mice, which has largely limited the functional characterization of MM cells and their interaction with the BM immune cells; and 2) the limited availability of experimental mouse models of MM that recapitulate the natural history of the disease. Among the available murine models are the 5TMM myeloma cells which can be transplanted into syngeneic mouse recipients, and the immunodeficient IL6 humanized mouse line that partially allows the growth of human MGUS and MM cells.

Among the genetically modified mice developing MM, only the Vk*MYC model fulfills some of the principal features of human MM, although it is focused on a single MYC genetic abnormality and its use requires syngeneic transplantation experiments to avoid the long latency (18 to 24 months) of disease onset (Chesi et al, Cancer Cell. 2008; 13(2):167-80) Because of recent MM clinical trials with immune-based agents have yielded different clinical outcomes, the use pre-clinical models to anticipate immunologic effects of the varied immunotherapies, to delineate mechanisms of resistance, and to identify optimal combinations would be a major step forward in MM therapy. Thus, the generation of valid experimental models of human-like MM represents an unmet medical need.

### SUMMARY OF THE INVENTION

The authors of the invention have developed genetically engineered mouse models of human-like MM that reflect the key elements in the pathogenesis of the disease: the genetic heterogeneity, the progressive transition of MGUS and SMM states into clinical active disease, and the interaction of tumor cells with the BM immune microenvironment during transformation.

In a first aspect, the invention relates to a genetically engineered mouse comprising a conditionally activatable transgene encoding a constitutively active form of the IKK2 protein and a transgene encoding the human BCL2 protein.

In a second aspect, the invention relates to a genetically engineered mouse comprising:
i) a conditionally activatable transgene encoding a constitutively active form of the IKK2 protein and a conditionally activatable transgene encoding the MYC protein,
ii) a conditionally activatable transgene encoding a constitutively active form of the IKK2 protein and a conditionally activatable transgene encoding the MMSET-II protein, or
iii) a conditionally activatable transgene comprising the MYC protein coding sequence and a conditionally activatable transgene encoding the MMSET-II protein.

In a third aspect, the invention relates to a bicistronic gene construct selected from the group consisting of:
i) a bicistronic gene construct comprising a first cistron that encodes a constitutively active form of the IKK2 protein and a second region that encodes the BCL2 protein,
ii) a bicistronic gene construct comprising a first cistron that encodes a constitutively active form of the IKK2 protein and a second cistron that encodes the MYC protein,
iii) a bicistronic gene construct comprising a first cistron that encodes a constitutively active form of the IKK2 protein and a second cistron that encodes the MMSET-II protein, and
iv) a bicistronic gene construct comprising a first cistron that encodes the MYC protein and a second cistron that encodes the MMSET-II protein; and
   wherein the gene construct is under operative control of a promoter and wherein an internal ribosomal entry site is present between the first and second cistrons.

In a fourth aspect, the invention relates to a vector comprising the bicistronic gene construct according to the third aspect of the invention.

In a fifth aspect, the invention relates to a genetically engineered mouse comprising the bicistronic gene construct of the third aspect of the invention or the vector of the fourth aspect of the invention integrated in its genome.

In a sixth aspect, the invention relates to a genetically engineered mouse according to the invention comprising within its genome the sequence encoding for a recombinase, as mentioned above, suffering from human-like multiple myeloma (MM).

In a seventh aspect, the invention relates to a method for the induction of multiple myeloma (MM) in a mouse according to the invention, comprising within its genome the sequence encoding for a recombinase, said method comprising maintaining the mouse under conditions adequate for the development of multiple myeloma.

In an eighth aspect, the invention relates to a genetically engineered mouse suffering from human-like MM which has been obtained by a method according to the seventh aspect of the invention.

In a ninth aspect, the invention relates to a MM cell population obtained from a mouse as defined in the eighth aspect of the invention.

In a tenth aspect, the invention relates to a method for the screening for a candidate substance for the treatment of a MM, which method comprises the steps of:
(i) providing a genetically engineered mouse according to the first or second aspect of the invention as far as they comprise within its genome a sequence encoding for a recombinase which is specific for the recombinase target sites flanking the transcription terminator sequence within the conditionally activatable transgene or transgenes and/or specific for the recombinase target sites present in the Trp53 gene region which is required for the expression of a functional Trp53 protein, or in a mouse according to fifth aspect of the invention as far as they additionally comprises within its genome a sequence encoding for a recombinase which is specific for the recombinase target sites flanking the transcription terminator sequence within the conditionally activatable cistron, and inducing in said mouse the proliferation of plasma cells until the presence of MM is detected, providing a mouse suffering from MM according to the sixth aspect of the invention or implanting into a mouse the MM cell population as defined in the ninth aspect of the invention under conditions adequate for the engraftment of said MM cell population;
(ii) administering said candidate substance to said mouse;
(iii) determining the effect of the candidate substance on the MM;
wherein an increased effect of the candidate compound on the MM with respect to the effect observed in a mouse treated with a control substance indicates that the candidate compound can be suitable for the treatment of MM.

In an eleventh aspect, the invention relates to the use of a genetically engineered mouse according to the first or second aspect of the invention as far as they comprise within its genome a sequence encoding for a recombinase which is specific for the recombinase target sites flanking the transcription terminator sequence within the conditionally activatable transgene or transgenes and/or specific for the recombinase target sites present in the Trp53 gene region which is required for the expression of a functional Trp53 protein, or in a mouse according to the fifth aspect of the invention as far as they additionally comprises within its genome a sequence encoding for a recombinase which is specific for the recombinase target sites flanking the transcription terminator sequence within the conditionally activatable cistron, a mouse suffering from MM according to the sixth aspect of the invention or implanting into a mouse the MM cell population as defined in the ninth aspect of the invention under conditions adequate for the engraftment of said MM cell population, for the screening a candidate substance for the treatment of a MM.

In a twelfth aspect, the invention relates to genetically engineered mouse comprising a conditionally activatable transgene encoding the human MMSET-II protein, wherein the conditionally activatable transgene comprise a transcription terminator sequence flanked by recombinase target.

### DESCRIPTION OF THE FIGURES

FIG. 1 Genetically heterogeneous mouse models of human-like multiple myeloma.
a) Schematic of the genetic screen strategy, whereby transgenic mice were crossed with cγ1-cre or mb1-cre mice. Among 31 genetically heterogeneous mouse lines generated, MImb1, MIcγ1 and BIcγ1 strains developed MM. GEM, genetically engineered mice; m, months.
b) Kaplan-Meier OS curves of MImb1, MIcγ1, BIcγ1, control (YFPcγ1 and YFPmb1) and Vk*MYC mice.
c) Representative flow cytometry analysis in the BM of BIcγ1 mice at the time of death, which shows an increased number of GFP+CD138+B220-sIgM-MM cells.
d) Giemsa staining of a representative BM sample in BIcγ1 mice revealed human-like PCs with expression of acid phosphatase (AP; left). On the right, immunohistochemical examination in BIcγ1 mice revealed CD138 surface expression by MM cells.
e) MM cells show increased surface expression of Bcma, Slamf7 and Taci according to flow cytometry analyses.
f) Representative electrophoresis of immunoglobulin secretion in serum samples from MImb1, MIcγ1 and BIcγ1 mice shows M spikes corresponding to the gamma fraction.
g) Quantification of immunoglobulin isotypes in serum samples by ELISA in MImb1 (n=3), MIcγ1 (n=2), BIcγ1 (n=4) and YFPcγ1 control (n=9) mice.
h) Kaplan-Meier survival curves of mouse lines that develop MM derived from the BIcγ1 strain with additional KRASG12D mutation, heterozygous Trp53 deletion, or expression of cyclin D1, c-MAF or MMSET.
i) Kaplan-Meier survival curves of mouse lines that develop MM derived from MIcγ1 mice with additional KRASG12D mutation, heterozygous Trp53 deletion, c-MAF expression or BCL2 expression.
j) Kaplan-Meier survival curves in mice with MMSET/NSD2 expression crossed with lines carrying either IKK2NF-κB activation or c-MYC expression, which developed MM at old ages.
k) Flow cytometry analyses in BIcγ1 and MIcγ1 mice revealed that precursor states precede clinically evident MM in genetically heterogeneous mice.
l) Analysis of Igh clonality according to RNA-seq of immunoglobulin gene loci and classification by the presence of explicit clonotypes for each sample. B cell receptor (BCR) repertoires and the most expanded clone groups in control, MGUS and MM samples. Log-rank (Mantel-Cox) test was used.

FIG. 2

Flow cytometry analyses of BM and spleen samples from genetically heterogeneous mice, YFP_{cγ1}/YFP_{mb1} control mice, and VK*MYC mice at 6 months of age. CSR, class-switch recombination. Strains marked with # developed mature B-cell lymphoma or acute lymphoblastic leukemia, as shown in FIG. 3. (*) In Vk*MYC mice, the percentage of plasma cells in the bone marrow and spleen correspond to total CSR cells without GFP or YFP expression.

FIG. 3

Nine strains developed B-cell lymphoma, plasmablastic lymphoma, and acute lymphoblastic leukemia.
a) Kaplan-Meier survival curves in two models that developed B-cell lymphoma: P53ₕₒₘI_{homcγ1} mice and P53ₕₒₘ/Kras-I_{homcγ1} mice.
b) Representative flow cytometry and immunohistochemical analyses in these strains.
c) Kaplan-Meier survival curves of three different models using the mb1-cre mice that developed T-cell acute lymphoblastic leukemia (left). Representative flow cytometry analyses of BM samples are shown on the right.

FIG. 4: Characterization of multiple myeloma in genetically engineered mice.
a) Examination of tumor clonality by genomic PCR and sequencing revealed clonal IgHV gene rearrangements in DNA isolated from BM PCs from MI_{mb1}, MI_{cγ1} and BI_{cγ1} mice. As negative control, peripheral blood CD19⁺ B cells from YFP_{cγ1} mice were included.
b) Representation of the fraction clonotype groups according to the tumor IgHV gene clonality in two samples from BI_{cγ1} and MI_{cγ1} mice at MGUS and MM states, shown on the left. The percentage of samples with clonal and non-clonal IgHV genes in BI_{cγ1}-derived and MI_{cγ1}-derived strains at MGUS and MM states is shown on the right.
c) Representation of CRAB features in MI_{mb1}, MI_{cγ1}, and BI_{cγ1} mice, including hypercalcemia, anemia, bone disease as revealed by reduced bone mineral density (BMD), and renal disease due to Ig light-chain deposits in tubules.
d) Representative examples of individual mice showing the quantification of Ig isotypes in serum samples by ELISA.

FIG. 5: Characterization of BIcγ1 and MIcγ1 models with additional genetic lessions.
a) Characterization of MM development in BI_{cγ1} strains carrying an additional Kras^{G12D} mutation or heterogeneous deletion of P53 (complementary to FIGS. 1h-i).
b) Characterization of MM development in MI_{cγ1} (b) strains carrying an additional Kras^{G12D} mutation or heterogeneous deletion of P53 (complementary to FIGS. 1h-i)
c) Characterization of MM development in MI_{cγ1} mice with additional expression of BCL2 (B-MI_{cγ1} mice) (complementary to FIG. 1i).

FIG. 6: Characterization of BIcγ1 and MIcγ1 mice with additional immunoglobulin chromosomal translocations.
a) Characterization of MM development in BI_{cγ1} mice carrying Ig chromosomal translocations (complementary to FIGS. 1h-i).
b) Characterization of MM development in MI_{cγ1} mice carrying Ig chromosomal translocations (complementary to FIGS. 1h-i).

FIG. 7
a) Characterization of MM development in mice with t(4; 14) crossed with lines carrying IKK2^{NF-κB} activation or MYC expression (complementary to FIG. 1j).
b) Quantification of GFP⁺CD138⁺B220⁻sIgM⁻ PCs by flow cytometry in the BM of BI_{cγ1} and MI_{cγ1} mice at MGUS and MM states, and in YFP_{cγ1} control mice at 6 months of age, are shown (complementary to FIG. 1k).
c) Representative electrophoresis analyses of Ig secretion in serum samples from BI_{cγ1} and MI_{cγ1} mice at MGUS and MM states, and in YFP_{cγ1} control mice, are shown.

FIG. 8

Description of the fifteen genetically heterogeneous mouse models of multiple myeloma. As a reference model, the Vk*MYC mice are included.

FIG. 9. Transcriptional and genomic profiling of multiple myeloma in mice.
a) RNA-seq analyses of typical PC and B cell genes in PCs from mice at MGUS (n=25) and MM (n=40) stages versus control BM PCs (n=6) and GC B cells (n=3). TPM, transcripts per million. Boxes represent the median, upper and lower quartiles and whiskers represent minimum to maximum range.
b) PCA of RNA-seq data from mouse and human MGUS and MM cells compared with control BM PCs. Human PCs were obtained from patients with newly diagnosed MGUS (n=9) and MM (n=41), and from BM aspirates from healthy donors (n=7).
c) PCA of RNAseq data from BIcγ1 and MIcγ1 mice revealed two transcriptional modes of evolution during MM development.
d) Quantitative PCR with reverse transcription (RT-qPCR) of mouse and human MYC gene expression in isolated BM PCs (n=7), MGUS (n=6) and MM (n=12) cells from BIcγ1-derived and MGUS (n=5) and MM (n=14) cells from MIcγ1-derived mice. The mean and s.d. are represented. Kruskal-Wallis test P values adjusted for multiple comparisons by Dunn's test are indicated.
e) GSEA of RNA-seq data shows ‘MYC target genes’ at the top of the MM hallmarks in BIcγ1-related and MIcγ1 mice. NES, normalized enrichment score.
f) Immunohistochemical image of BM sections revealed nuclear MYC protein expression in GFP+ MM cells from BIcγ1 mice (left). Western blot analysis revealed MYC expression in mouse MM-derived cell lines (right).
g) MYC expression from RNA-seq data in samples from patients with MGUS (n=8) or MM (n=39) and in BM PCs (n=7) from healthy donors. The mean±s.d. is represented. Western blot analysis of MYC protein expression in human MM cell lines (right).
h) Representative examples of spectral karyotyping analysis in metaphase cells from two MM-derived cell lines.
i) Copy number variation and WES analyses of primary cells from mice with MGUS and MM and in an MM-derived cell line.
j) WGS mapped the breakpoints in two chromosomal translocations between the Igh or Igl and MYC genes in MM9275 and MM5080 cell lines, respectively.
k) MYC targeting with the MYC inhibitor MYCi975 reduced MYC expression (right) and decreased MM cell viability (left) in mouse and human MM cells. Data corresponding to the mean±s.e.m. from two or three independent experiments are represented for each cell line. *P<0.05; **P<0.01; ***P<0.001.

FIG. 10
a) PCA of murine MM transcriptomes, which separates MM cells (n=38) from GC B cells (n=3) and PCs (n=6). The samples used are those characterized in FIG. 9a.
b) Unsupervised clustering analyses of the RNA-seq data corresponding to these samples, also including MGUS samples (n=24). DEGs, differentially expressed genes.
c) Gene set enrichment analysis (GSEA) showing enrichment of mouse deregulated genes (down and up regulated) in the human MM transcriptome. NES, normalized enrichment score

FIG. 11

Representation of the procedure to establish MM cell lines from primary mouse MM cells.

FIG. 12

CNV in 62 murine samples from the MM stage, 3 samples of pooled PCs from 9 mice at the MGUS stage, and 6 samples from MM-derived cell lines. Six BM samples with YFP⁺ CD138⁺B220⁻ PCs isolated from YFP_{cγ1} mice were also included. In addition, the 5TGM1 and 12598-Vk*MYC cell lines were analyzed.

FIG. 13. A common MAPK-MYC axis dictates multiple myeloma progression.
a) Quantification of the TMB, which corresponds to the total number of somatic mutations per tumor, according to WES analysis (left). Distribution of mutations in genes within signaling and cancer-related pathways in MM (n=62) and MGUS (n=3) primary samples, and in MM-derived cell lines (n=6). Kruskal-Wallis test P values adjusted for multiple comparisons by Dunn's test are indicated.
b) Quantification of copy number variation and TMB according to WES data from MM cells from Trp53-BIcγ1 mice compared with the remaining strains, and in MM patients from the CoMMpass study with and without 17p/TP53 deletion and/or TP53 somatic mutations. Mann-Whitney test two-tailed P values are indicated.
c) Western blot analyses revealed ERK phosphorylation in mouse and human MM cell lines. The mouse cell line 5TGM1 was included as a positive control.
d) The MEK inhibitor trametinib induced a dose-dependent reduction in ERK phosphorylation in mouse and human MM-derived cell lines.
e) Dose-dependent decrease in viability of mouse and human MM cell lines following trametinib treatment. Data corresponding to the mean±s.e.m. from two to ten independent experiments are represented for each cell line.
f) Reduced phosphorylation of MYC at S62 (pMYC-S62) following treatment with trametinib in mouse and human MM cell lines. Quantification of the fold change in expression levels of pMYC-S62 with respect to total MYC protein is shown.

Boxes represent the median, upper and lower quartiles and whiskers represent minimum to maximum range (a and b). **P<0.01; ***P<0.001.

FIG. 14 Immune features of multiple myeloma progression.
a) Distribution of lymphoid cell subpopulations in the BM of mice with MGUS and MM, and in control mice.
b) Two-tailed Pearson correlation analyses between the number of BM PCs in mice at MGUS and MM states with T cells or NK cells in the BM.
c) Classification of MM samples into categories according to the abundance of T and NK lymphoid cells in the BM with respect to that in healthy mice.
d) MM cases with higher number of infiltrating immune cells contained more tumorreactive PD-1+CD8+ T cells and Treg cells. Two-tailed Pearson correlation analysis between CD8+ T cells and Treg cells in the BM (right).
e) Characterization of the BM lymphoid cell composition by flow cytometry in BM samples from patients with MGUS, SMM and MM.
f) Two-tailed Pearson correlation analyses between the percentage of PCs in the BM from MM patients and the percentage of T or NK cells in the BM.
g) Classification of MM patients (n=652) into those with lower and higher number of immune cells in the BM microenvironment with respect to healthy donors (HDs; n=24).
h) Tumors with high immune infiltrates contained more tumor-reactive PD-1+CD8+ T cells and Treg cells in the BM compared with MM cases with a lower number of immune cells. Two-tailed Pearson correlation analysis between the percentages of CD8+ T cells in BM and the percentage of Treg cells (right).
i) MM cases with more abundant immune cells had increased immunoglobulin secretion with respect to the remaining cases.
j) Two-tailed Pearson correlation analyses between the T and NK lymphoid cell infiltrate in BM from mice (n=59) and humans with MM (n=638) and the age.
k) Quantification of the BM lymphoid infiltrates including CD4+, CD8+ and NK cells across genetic subgroups of mouse and human MM. Boxes represent the median, upper and lower quartiles and whiskers represent minimum to maximum range (a, d, e, h, i and k). Kruskal-Wallis test P values adjusted for multiple comparisons by Dunn's test (a, b and k) and Mann-Whitney test P values (d, h and i) are indicated. *P<0.05; **P<0.01; ***P<0.001.

FIG. 15: Immunological characteristics of genetically engineered mice with multiple myeloma.
a) Phenotype of CD8⁺ T cells in the BM during progression in mice, which acquired activated phenotypes and expressed exhaustion markers compared with control age-matched mice (complementary to FIG. 15a).
b) Phenotype of CD4⁺ T cells during MGUS and MM progression in mice. Controls corresponded to YFP_{cg1} mice (complementary to FIG. 14a).
c) Percentage of NK cells with TIGIT and LAG3 expression in BM in YFP_{cg1} control mice and in mice with MGUS and MM (complementary to FIG. 14a).
d) Immune-inflamed tumors contained an increased number of tumor-reactive CD8⁺ T cells that expressed PD1, TIGIT, and LAG3, in contrast to immune-cold MM cases (complementary to FIG. 14d).
e) Pearson correlation analyses between the percentages of NK cells in the murine BM and those of T_{reg} cells in the BM (complementary to FIG. 14d).
f) In MM patients, immune-inflamed tumors contained an increased number of tumor-reactive CD4⁺ T cells and NK cells in the BM compared with immune-cold MM cases (complementary to FIG. 14g).
g) Pearson correlation analyses between the percentages of NK cells in the BM of MM patients and those of T_{reg} cells in the BM (complementary to FIG. 14h).

FIG. 16: Bio-informatic deconvolution of RNAseq data.

Bio-informatic deconvolution of RNA-seq data was applied to a previously reported clinical series of 72 newly diagnosed MM patients (GSE104171), which allowed the definition of the cellular composition of the BM microenvironment34,35. a) These studies confirmed the presence of the MM immunological subgroups, which divided the patients into two immune categories according to the abundance of immune cells in the BM. b) In this clinical series, patients with abundant immune cells MM (28 cases, 39%) presented higher number of PD-1+CD8+ T cells and Treg cells with respect to those patients with low-infiltrating MM cases (44 cases, 61%). c) In addition, the transcriptomic signatures corresponding to CD8+ T cells, Treg cells, NK cells and Thelper type 1 and type 2 cells were increased in the cases with higher number of immune cells with respect to those with less abundant T and NK cells.

FIG. 17: Immunological characterization of genetically heterogeneous mouse and human multiple myeloma.
a) MM with lower frequency of immune cells was more common in patients older than 70 years.
b) Measurement of the tumor mutation burden in MM samples from mice (n=24) and patients (n=24) according to whole-exome sequencing (WES) studies of somatic mutations.
c) Pearson correlation studies of the TMB quantified and the T and NK cells infiltrating the BM in murine MM.
d) Pearson correlation studies of the TMB quantified and the T and NK cells infiltrating the BM in human MM.

FIG. 18. Immunotherapy responses in multiple myeloma
a) Preclinical immunotherapy trial in MIcγ1 mice testing anti-PD-1 or anti-TIGIT monoclonal antibodies with respect to isotype-treated mice. Kaplan-Meier OS curves and mOS values are shown.
b) Preclinical immunotherapy trial in BIcγ1 mice testing anti-PD-1 or anti-TIGIT monoclonal antibodies with respect to isotypetreated mice. Kaplan-Meier OS curves and mOS values are shown.
c) MIcγ1 mice (n=10) exhibited higher numbers of activated PD-1+, TIGIT+ and LAG3+CD8⁺ T lymphocytes in the BM compared with BIcγ1 mice (n=9).
d) The number of PD-1+ Treg cells in the BM of MIcγ1 mice (n=9) was lower than in BIcγ1 mice (n=9) at MGUS stages.
e) The ratio of CD8+ T cells to Treg cells in the BM microenvironment was higher in MIcγ1 mice (n=8) than in BIcγ1 mice (n=9; median value, 22.5 versus 6.1; P=0.019).
f) Representation of the CD8+ T/Treg cell ratio in BM samples from mouse MM and from patients with SMM. Median value of CD8+ T/Treg cell ratios in SMM patients with progression versus those without progression at 2 years from diagnosis (P<0.05; right).
g) Kaplan-Meier PFS curve for patients with untreated SMM (n=69). A high CD8+ T/Treg cell ratio was associated with shorter time to progression with respect to the remaining cases (median PFS at 2 years, 38% versus 88%; P=0.005).
h) In 170 newly diagnosed individuals with clinically active MM, 23(14%) exhibited a high CD8+ T/Treg cell ratio, while the remaining patients (86%) showed lower CD8+ T/Treg cell ratios.
i) Kaplan-Meier PFS curve for 170 MM patients aged >70 years treated with lenalidomide and dexamethasone in the GEM-CLARIDEX clinical trial (NCT02575144). The presence of a high BM CD8⁺ T/Treg cell ratio was associated with a higher rate of progression in comparison with those cases with low values (PFS, 18 months versus not reached; P=0.011). Boxes represent the median, upper and lower quartiles and whiskers represent minimum to maximum range (c-f). Unpaired two-tailed Student's t-test or Mann-Whitney test P values (c-f) are indicated. Log-rank (Mantel-Cox) test was used in a, b, g and i. *P<0.05; **P<0.01; ***P<0.001.
j) Uniform manifold approximation and projection (UMAP) plots of single-cell transcriptomic and TCR genomic profiles from CD8+ T cells and Treg cells in mice and patients at MM states are shown. In mice and humans, cells with a clonotypic TCR were identified preferentially among the CD8+ T cell subset.
k) In vivo depletion of CD4+ or CD8+ T cells in the MM5080 syngeneic transplantation model is shown. The Kaplan-Meier OS curve included two experiments. The mOS and the number of mice in each treatment cohort are shown. In vivo genetic depletion of Treg cells in Foxp3-GFP-DTR mice with transplanted MM5080 cells. The mOS and the number of mice in each treatment cohort are shown.
l) Enhancing CD8+ T cell cytotoxicity by TIGIT co-inhibition dictates anti-PD-1 responses. The mOS and the number of mice in each treatment cohort are shown.
m) Depletion of Treg cells with a mouse anti-CD25 monoclonal antibody delayed MM onset and increased anti-PD-1 responses. The mOS and the number of mice in each treatment cohort are shown. Log-rank (Mantel-Cox) test was used. *P<0.05; **P<0.01; ***P<0.001.

FIG. 19
a) Comparison of the BM immune phenotypes including the number of activated PD1⁺TIGIT⁺LAG3⁺CD8⁺ T lymphocytes at MM stages in MI_{cγ1} vs. BI_{cγ1} mice (complementary to FIG. 18c).
b) Comparison of the number of PD1⁺ T_{reg} cells in the BM of MI_{cγ1} mice and in BI_{cγ1} mice (complementary to FIG. 18c).

FIG. 20
a) Kaplan-Meier survival curves in MI_{cγ1} mice undergoing depletion of CD4⁺ and CD8⁺ T cells. Monoclonal antibodies were administered by i.p. injection when MI_{cγ1} and BI_{cγ1} mice were 4 and 6 months of age, respectively. Mice received 100 μg of anti-CD4, anti-CD8, or rat IgG control antibodies, administered on days +1, +4, and +8 and then weekly for 8 weeks. Median overall survival, mOS. The number of mice included on each cohort is represented.
b) Similar study was conducted in BIcγ1 mice. In the figure, Kaplan-Meier survival curves are shown.

FIG. 21: Immunological characteristics of mouse and human multiple myeloma.

Bulk RNA-seq and microarray data analyses in mouse and human MM. The composition of the BM microenvironment was next investigated in the clinical study of MM patients by applying bio-informatic TME reconstruction to RNA-seq and microarray data from BM samples.

Those patients with a TME overlapping with that of MIcγ1 mice exhibited superior outcome under lenalidomide/thalidomide-containing regimens compared with the remaining cases (median PFS, 85 months vs. 62 months; p=0.0005) (complementary to FIG. 18i).

FIG. 22: Functional evaluation of immunological features in mouse models of multiple myeloma.

Single-cell RNA-seq and TCR-seq analyses in mouse and human samples at MGUS stage, and in healthy mice and humans (complementary to FIG. 18j). Uniform manifold approximation and projection (UMAP) plots of single-cell transcriptomic and TCR genomic profiles are shown.

FIG. 23: Modulating CD8+ T/Treg cell ratio enhances immunotherapy outcomes a) scRNA-seq/TCR-seq analyses of 60,858 CD3+ T cells isolated from the BM of MIcγ1 and BIcγ1 mice, and from YFPcγ1 controls. Three mice from each subgroup at MGUS and MM states were included. In patients, scRNA-seq/TCR-seq analyses of 50,154 CD3+ T cells isolated from the BM of newly diagnosed MM (n=7) and MGUS (n=4), and from the BM of healthy adults (n=6), were performed.
b) Quantification of the expression of selected markers in CD8⁺ T cells in MIcγ1 and BIcγ1 mice at different disease states (left). Quantification of the expression of markers in CD4+CD25+Foxp3+ T cells in MIcγ1 and BIcγ1 mice and in MM patients at MGUS and MM stages (right).

FIG. 24: Syngeneic mouse models of multiple myeloma.

Characterization of the BM immune phenotype in syngeneic transplants in C57BL/6 mice generated from the MM5080 cell line established from P53-BIcγ1 mice.
a) Flow cytometry analysis in BM samples obtained at the time of mouse death (from day +20 to +35 post i.v. injection of tumor cells). Quantification of MM cells, CD4⁺ and CD8⁺ T lymphocytes, and NK cells in syngeneic and control C57BL/6 mice is shown.
b) Syngeneic transplants showed higher number of immunosuppressive PD-1⁺ T_{reg} cells with respect to control C57BL/6 mice.
c) Characterization of CD4⁺ T lymphocytes in syngeneic transplants vs. control C57BL/6 mice.
d) Characterization of CD8⁺ T lymphocytes in syngeneic transplants vs. control C57BL/6 mice.

FIG. 25

Syngeneic transplants from the MM5080 cell line were refractory to therapies with moAbs that inhibit PD-1, PD-L1 and TIGIT. Monoclonal antibodies (200 μg) were administered by i.p. injection twice weekly for 3 weeks starting on day +1 post-transplantation. Mice received 200 μg of anti-PD1, anti-PD-L1, anti-TIGIT, or rat IgG control antibody. In the control group, 100 μg of rat IgG antibodies were similarly administered. Therapy responses were determined by comparing median overall (mOS) in Kaplan-Meier survival curves. The number of mice included on each cohort is represented.

FIG. 26

Simultaneous inhibition of PD-L1 and TIGIT increased survival in a fraction of treated mice. 200 μg of anti-TIGIT, anti-PDL1, and both moAbs were i.p. injected twice weekly for 3 weeks starting on day +1 post-transplantation. In the control group, 100 μg of rat IgG antibodies were similarly administered. The number of mice included on each cohort is represented. Therapy responses were estimated by Kaplan-Meier survival curves, which were compared using the log-rank test.

FIG. 27

Depletion of T_{reg} cells with the anti-CD25 moAb combined with inhibition of PD-L1 efficacy decreased MM growth in the subcutaneous MM8273 syngeneic model. Therapy was started when tumors reached 400 mm³. 200 μg of anti-PDL1 moAb were i.p. injected twice weekly for 3 weeks starting on day +1 post-injection. Anti-CD25 antibody was administered by i.p. injection starting on day +1 post-injection (75 μg/mouse) and continued weekly for three consecutive weeks. Tumor growth was monitored every two days by measuring tumor size in two orthogonal dimensions using a caliper. Tumor volume was calculated using the formula V=(L²×W)/2.

FIG. 28. Mouse Model of MM with Humanized CRBN
a) Splenocytes isolated from wild-type and humanized-hCRBN^{I319V} mice were incubated with different lenalidomide concentrations ex vivo for 24 h. Quantification of Ikaros expression was performed by Western blot.
b) CD3 T cells were purified from wild-type and hCRBN^{I319V} mice and cultured for 24 h in the presence of lenalidomide or pomalidomide. Ikaros degradation and IL2 production were evaluated by Western blot and ELISA, respectively, at the indicated doses.
c) Schematic representation of MI_{cγ1} mice and of MI_{cγ1} with the humanized CRBN transgene.
d) MI_{cγ1}^{hCRBN} mice progressively developed an expansion of tumor plasma cells in bone marrow that remodeled the local microenvironment. In the figure, measurement of the percentage of bone plasma cells, CD4 and CD8 T cells, and NK cells in MI_{cγ1} and MI_{cγ1}^{hCRBN} mice, which exhibited comparable results.

FIG. 29. Treatment of humanized CRBN mice with immunomodulatory drugs.
a) MI_{cγ1}^{hCRBN+/-} mouse was treated for 4 weeks with lenalidomide (30 mg/kg); splenocytes and bone marrow cells from sacrificed mice showed Ikaros degradation.
b) Kaplan-Meier overall survival curve of MI_{cγ1}^{hCRBN+/+} mice, which received lenalidomide (R) or the combination of lenalidomide plus bortezomib and dexamethasone (RVd) during eight weeks. A major increase in the survival rate of the two treatment arms is observed in comparison to control mice (p<0.001). The number of mice included in each cohort is shown.
c) Representative analysis of serum electrophoresis in control, R and RVd treated mice at different timepoints during therapy.
d) Quantification of serum immunoglobulins in MI_{cγ1}^{hCRBN+/+} in response to drug administration compare to control arm (left). Waterfall plot showing the M-spike fold change eight weeks after therapy initiation (right).

FIG. 30. Key resource table that includes all the reagents and materials used in this study.

FIG. 31. Schematic representation of the bi-cistronic vectors engineered for the generation of mouse models of multiple myeloma.

### DETAILED DESCRIPTION OF THE INVENTION

### Mouse Models of Human Myeloma

In first aspect the invention relates to a genetically engineered mouse comprising a conditionally activatable transgene encoding a constitutively active form of the IKK2 protein and a transgene encoding the human BCL2 protein.

In a second aspect, the invention relates to a genetically engineered mouse comprising:
i) a conditionally activatable transgene encoding a constitutively active form of the IKK2 protein and a conditionally activatable transgene encoding the MYC protein,
ii) a conditionally activatable transgene encoding a constitutively active form of the IKK2 protein and a conditionally activatable transgene encoding the MMSET-II protein, or
iii) a conditionally activatable transgene comprising the MYC protein coding sequence and a conditionally activatable transgene encoding the MMSET-II protein.

The term “genetically engineered mouse” refers to mice whose genome has been altered by genetic engineering, or their offspring. Typically, a genetically engineered may be either a transgenic mouse or a chimeric mouse.

The term “transgenic mouse” is defined as a mouse at least some of whose germ cells contain genetic material, originally derived from another mouse, other than an ancestor of said mouse, as a result of human intervention. So defined, it includes progeny of a transgenic mouse which retain the transgenic genotype. It is not necessary that all cells of the mouse contain the transgene.

The reference to human intervention is intended to exclude genetic modification as a result of unintentional infection with a virus.

The term “chimeric mouse” is defined, as a mouse which is not necessarily a transgenic mouse, but at least some of whose somatic cells contain genetic information, originally derived from another mouse other than an ancestor of said mouse, as a result of human intervention.

Note that mice produced by conventional artificial insemination techniques are not considered to be genetically engineered, the donors of sperm and egg being considered parents of the mouse, unless one or more ancestors of the mouse was genetically engineered and the descendant mouse retains the engineered genotype. Moreover, the transplantation of cells from one mouse to another is not considered genetic engineering.

The term “conditionally activatable transgene” or “conditionally active transgene”, in accordance with the present invention, refers to a nucleic acid sequence that has been introduced into the genome in such a way that it is not expressed constitutively. In an embodiment, only upon recombinase-mediated removal of the selection marker cassette, which is located 5′ of the transgene, does the transgene become expressed by means of the endogenous promoter. It is therefore possible to activate transgene expression in a time- and tissue-specific manner, for example by introducing the recombinase into the genome of the mouse comprising the conditionally activatable transgene by means of genetic engineering or by crossing a transgenic mouse comprising the conditionally activatable transgene with a second transgenic mouse expressing the respective recombinase in the tissue and/or at the time of interest.

“Promoter” means a minimal sequence sufficient to direct transcription. Also included in the invention are those promoter elements which are sufficient to render promoter-dependent gene expression controllable for cell-type specific or tissue-specific regulators; or inducible by external signals or agents; such elements may be located in the 5′ or 3′ regions of the native gene. A promoter element may be positioned for expression if it is positioned adjacent to a DNA sequence so it can direct transcription of the sequence.

The term “transgene”, in accordance with the present invention, refers to a nucleic acid sequence that is introduced into the genome of the cell. In non-limiting examples, the transgene can consist of an exogenous gene not normally present in the target sequence, such as for example a gene from one species that is introduced into a cell derived from another species. In a further non-limiting example, the transgene can be essentially identical to the part of the genome but carrying a disease-causing mutation or, alternatively, the transgene can be a gene compensating for the lack of a gene.

A number of different strategies for the modification of the genome, and in particular the mouse genome, are used so far. One exemplary aspect is the introduction of disease-mediating mutations or entire disease-mediating genes into the genome of a mouse model. Most of the methods for achieving this involve the introduction of transgenes into the genome as well as the use of homologous recombination (HR) techniques for targeted gene modifications or the use of non-targeted gene trapping.

For the generation of traditional transgenic mice, genes responsible for particular traits or disease susceptibilities are chosen and extracted and are injected into fertilized mouse eggs. Embryos are implanted in the uterus of a surrogate mother and the selected genes will be expressed by some of the offspring. These conventional transgenic approaches offer the advantage that they are relatively straightforward and inexpensive. In addition, high levels of target gene expression can be achieved, and transgenic overexpressing mice, such as for example mice, often demonstrate obvious phenotypes. However, the site of integration as well as the copy number of the transgene in the genome can seriously affect tissue specificity and levels of transgene expression (Schonig et al., 2002). In particular, the site of integration is generally random, thus not allowing for a targeted modification of the genome.

Gene modification via HR is based on the targeted insertion of a selectable marker (often the neomycin phosphotransferase gene, neo) into an exon of the target gene, the replacement of one or more exons or, alternatively, the insertion of additional nucleic acid sequences into a target locus. The mutant allele is initially assembled in a specifically designed gene targeting vector such that the sequence to be inserted is flanked at both sides with genomic segments of the target gene that serve as homology regions to initiate homologous recombination. Upon the isolation of recombinant embryonic stem (ES) cell clones, modified ES cells are injected into blastocysts to transmit the mutant allele through the germ line of chimeras and to establish a mutant strain. (Hasty P, Abuin A, Bradley A., 2000, In Gene Targeting: a practical approach, ed. AL Joyner, pp. 1-35. Oxford: Oxford University Press; Nagy A, Gertsenstein M, Vintersten K, Behringer R., 2003. Manipulating the Mouse Embryo. Cold Spring Harbour, New York: Cold Spring Harbour Laboratory Press).

To study gene function only in specific cell types, conditional gene targeting schemes allow gene inactivation in specific cell types or developmental stages. In conditional mutants, gene inactivation may be achieved by the insertion of two recombinase recognition sites (RRS) for a site-specific DNA recombinase into introns of the target gene such that recombination results in the deletion of the RRS-flanked exons. As mentioned above, conditional mutants may require the generation of two mouse strains: one strain harboring a RRS flanked gene segment obtained by gene targeting in ES cells and a second, transgenic strain expressing the corresponding recombinase in one or several cell types, thus, wherein the conditional mutant is generated by crossing these two strains such that target gene inactivation occurs in a spatial and temporal restricted manner, according to the pattern of recombinase expression in the second transgenic strain. Or alternatively, the recombinase may be inserted and therefore expressed in the same mouse harboring the RRS flanked gene segment.

The methods for evaluating the presence of the introduced transgene as well as its expression are readily available and well-known in the art. Such methods include, but are not limited to, DNA (Southern) hybridization to detect the exogenous DNA, polymerase chain reaction (PCR), polyacrylamide gel electrophoresis (PAGE) and blots to detect DNA, RNA or protein.

The term “constitutive” or “constitutively active” includes a state in which a gene product is produced in a living cell in all or most of the physiological conditions of the cell.

For the generation of the genetically engineered mouse, it is first required to produce a cell comprising a conditionally active transgene in its genome, thus, a targeting vector needs to be inserted into genome of the cell.

In accordance with the present invention, the cell is selected from the group consisting of an embryonic stem cell, an induced pluripotent stem cell (iPS), a primordial germ cell or a somatic cell.

The term “embryonic stem cells”, as used throughout the present invention, refers to stem cells derived from the inner cell mass of an early stage embryo known as a blastocyst. ES cells are pluripotent, i.e. they are able to differentiate into all derivatives of the three primary germ layers: ectoderm, endoderm, and mesoderm. Recent advances in embryonic stem cell research have led to the possibility of creating new embryonic stem cell lines without destroying embryos, for example by using a single-cell biopsy similar to that used in preimplantation genetic diagnosis, which does not interfere with the embryo's developmental potential (Klimanskaya et al. (2006)). Furthermore, a large number of established embryonic stem cell lines are available in the art (according to the U.S. National Institutes of Health, 21 lines are currently available for distribution to researchers), thus making it possible to work with embryonic stem cells without the necessity to destroy an embryo. In a preferred embodiment, the embryonic stem cells are non-human embryonic stem cells.

“Induced pluripotent stem (iPS) cells”, in accordance with the present invention, are pluripotent stem cell derived from a non-pluripotent cell, typically an adult somatic cell, by inducing a “forced” expression of certain genes. Induced pluripotent stem cells are identical to natural pluripotent stem cells, such as embryonic stem cells in many respects, such as the expression of certain stem cell genes and proteins, chromatin methylation patterns, doubling time, embryoid body formation, teratoma formation, viable chimera formation, and potency and differentiability. Induced pluripotent stem cells are an important advancement in stem cell research, as they allow researchers to obtain pluripotent stem cells without the use of embryos (Nishikawa et al. (2008)). The induced pluripotent stem cells may be obtained from any adult somatic cells, preferably from fibroblasts, e.g. from skin tissue biopsies.

The term “primordial germ cells”, as used herein, refers to precursor germ cells which have not yet reached the gonads where they mature into sperm or ova as well as to mature spermatozoa and ova. Methods for the culturing of primordial germ cells including suitable media are well established in the art. For example, primordial germ cells may be differentiated in vitro from ES cells, such as for example the above recited ES cells and established ES cell lines. Also, iPS cells can be used as a starting cell for the differentiation of primordial germ cells (Park et al. 2009). In a preferred embodiment, the primordial germ cell is a non-human primordial germ cell.

The term “somatic cells”, as used herein, refers to any cell type in the mammalian body apart from germ cells and undifferentiated or partially differentiated stem cells.

Preferably, the somatic cell is a cell from which induced pluripotent stem cells can be derived.

A conditional transgenic mouse, in accordance with the present invention, refers to a mouse carrying a transgene in its genome, wherein the expression of the transgene can be activated in a tissue- and time-dependent manner.

The terms “nucleic acid molecules” as well as “nucleic acid sequences”, as used throughout the present description, are used according to the definitions provided in the art and include DNA, such as cDNA or genomic DNA, and RNA, such as mRNA.

The terms “polypeptide” and “peptide” are used interchangeably herein to refer to polymers of amino acids of any length. The polymer can be linear or branched, it can comprise modified amino acids, and it can be interrupted by non-amino acids.

The term “amino acid” refers to naturally occurring and synthetic amino acids, as well as amino acid analogs and amino acid mimetics that function in a manner similar to the naturally occurring amino acids. Furthermore, the term “amino acid” includes both D- and L-amino acids (stereoisomers).

“Encoding” includes the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if, for example, transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “IKK2”, also known as “IKK-β”, “I-Kappa-B-Kinase Beta” or “I-Kappa-B-Kinase 2” is used to refer to an inhibitor of nuclear factor kappa-B kinase subunit beta. IKK-β is an enzyme that serves as a protein subunit of IκB kinase, which is a component of the cytokine-activated intracellular signaling pathway involved in triggering immune responses. In a particular embodiment, the IKK2 of the invention is any of the disclosed in Calado D P et al., Cancer Cell. 2010 Dec. 14; 18(6):580-9.

The expression “constitutively active form of the IKK2 protein” refers to an IKK2 enzyme which is continually activated regardless of whether there are activation signals or stimuli. In a preferred embodiment, the active form of the IKK2 is a mutant protein comprising two serine to glutamate substitutions (S177E, S181E) in the activation loop of the kinase domain.

MYC, also known as Transcription Factor P64 or Proto-Oncogene C-Myc protein, is as defined in Sander S, et al., Cancer Cell. 2012 Aug. 14; 22(2):167-79. doi: 10.1016/j.ccr.2012.06.012. PMID: 22897848

The term “BCL2 protein”, also known as “PPP1R50” or “2, apoptosis regulator”, refers to an integral outer mitochondrial membrane protein that blocks the apoptotic death of some cells such as lymphocytes. Constitutive expression of BCL2, such as in the case of translocation of BCL2 to Ig heavy chain locus, is thought to be the cause of follicular lymphoma. Alternative splicing results in multiple transcript variants, which may all be considered as included within the present invention. In a particular embodiment the BCL2 protein is the human BCL2 protein, which gene is shown in the Ensembl database under accession number ENSG00000171791 (Ensemble release 106, April 2022). In a particular embodiment, the BCL2 protein is the one as disclosed in Strasser A, et al., Proc Natl Acad Sci USA. 1991 Oct. 1; 88(19):8661-5.

The MMSET protein, also known as WHS, WHSC1, NSD2 (Probable histone-lysine N-methyltransferase), WHSC2 or KMT3G, refers to an enzyme that contains four domains present in other developmental proteins: a PWWP domain, an HMG box, a SET domain, and a PHD-type zinc finger.

In a particular embodiment, the MMSET protein is the human MMSET protein, more particularly the protein encoded by the gene shown in the Ensembl database under accession number ENSG00000109685 (Ensemble release 107, July 2022). In a more particular embodiment, the MMSET protein is the long isoform II.

In a particular embodiment, when the genetically engineered mouse comprises a conditionally activatable transgene encoding a constitutively active form of the IKK2 protein and a conditionally activatable transgene encoding the MYC protein, then the genetically engineered mouse further comprises at least one of the following:
a conditionally activatable transgene encoding the K-Ras^{G12D} protein,
a inTrp53 gene,
a transgene encoding the BCL2 protein, or
a transgene encoding the MAF protein.

As used herein, the term “K-Ras protein” also known as “Kras protein” or “Kirsten rat sarcoma virus protein” is protein that is a member of the small GTPase superfamily. K-Ras acts as a molecular on/off switch, using protein dynamics. The amino acid positions that account for the overwhelming majority of these mutations are G12, G13 and Q61. A single amino acid substitution is responsible for an activating mutation. The transforming protein that results is implicated in various malignancies, including lung adenocarcinoma, mucinous adenoma, ductal carcinoma of the pancreas and colorectal carcinoma. Alternative splicing leads to variants encoding two isoforms that differ in the C-terminal region.

The term “K-Ras^{G12D}” refers to the K-Ras protein in which the glycine (G) at position 12 had been substituted with an aspartic acid (D). This mutation triggers structural, conformational and dynamic changes that result in constitutive activation of the protein (Vatansever S el al., Sci Rep. 2019 Aug. 13; 9(1):11730).

The term Trp53 gene, as used herein, refers to a gene encoding the tumor suppressor protein p53 which contains transcriptional activation, DNA binding, and oligomerization domains. The “tumor suppressor protein p53”, also known as “cellular tumor antigen p53” (UniProt name), or “transformation-related protein 53 (TRP53)”, is any isoform of a protein encoded by homologous genes in various organisms, such as TP53 (humans) and Trp53 (mice). This homolog (originally thought to be, and often spoken of as, a single protein) is crucial in vertebrates, where it prevents cancer formation. In particular embodiment, the Trp53 protein is the one disclosed in Marino S, et al., Genes Dev. 2000 Apr. 15; 14(8):994-1004.

The term “conditionally inactivatable” means that the gene function can be inactivated only in certain tissues or at certain times.

Different methodologies are known in the art for inactivating the gene expression in a mouse. For example, by altering the genome at specific gene loci through homologous recombination in mouse embryonic stem (ES) cells. The recombination may lead to conditional gene deletion. The conditional gene deletion may be achieved by the Cre-loxP system in which the first step is to prepare a mouse model in which either the whole gene of interest or a critical gene segment is flanked by two loxP sites. The conditional mouse lines containing two loxP sites are usually designed to be wild-type mice. Because of such requirements, the selection of the sites to be inserted by loxP sequences is important. Usually, large introns with fewer regulatory elements are preferred; non-coding sequences that are conserved among species should be left intact (Sharma S, Zhu J. Immunologic applications of conditional gene modification technology in the mouse. Curr Protoc Immunol. 2014; 105:10.34.1-10.34.13. Published 2014 Apr. 2).

The term MAF, also known as Proto-oncogene c-Maf, Transcription factor Maf-2 or V-maf musculoaponeurotic fibrosarcoma oncogene homologprotein refers to a DNA-binding, leucine zipper-containing transcription factor that acts as a homodimer or as a heterodimer. In particular embodiment, the MAF protein is the one disclosed in Morito N, et al., Cancer Res. 2011 Jan. 15; 71(2):339-48.

In another embodiment, when the genetically engineered mouse comprises a conditionally activatable transgene encoding a constitutively active form of the IKK2 protein and a transgene encoding the human BCL2 protein, then it further comprises at least one of the following:
a conditionally activatable transgene encoding the K-Ras^{G12D} protein,
a conditionally inactivatable Trp53 gene,
a transgene encoding the Cyclin-D1 protein,
a transgene encoding the MAF protein,
a conditionally activatable transgene encoding the MMSET-II protein, or
a conditionally activatable transgene encoding the MYC protein.

The term “Cyclin-D1”, also known as CCND1, BCL1, D11S287E, PRAD1 or U21B31 is a protein which belongs to the highly conserved cyclin family, whose members are characterized by a dramatic periodicity in protein abundance throughout the cell cycle. Cyclins function as regulators of CDK kinases.

In a particular embodiment, the Cyclin-D1 protein is the one disclosed in Katz S G, et al., Blood. 2014 Feb. 6; 123(6):884-93.

In a preferred embodiment, the constitutively active form of the IKK2 protein contains the S177E and S181E mutations.

In another embodiment, the transgene encoding the constitutively active form of the IKK2 protein is inserted in the Rosa26 locus.

By “ROSA26 locus” is used herein to refer to a locus located in mouse chromosome 6 and having the sequence corresponding to NCBI accession number FW565793.1 (Dec. 27, 2010). The ROSA26 produces three transcripts (FIG. 1): two transcripts originate from a common promoter share identical 5′ ends (exon 1 and exon 2 start), but neither contains a significant ORF. And a third one originated from the reverse strand. The Rosa26 locus is expressed in ES cells and many derivative tissues both in vitro and in vivo and new genetic material can be easily introduced into it through homologous recombination. WO2008088863A2.

In another embodiment, the genetically engineered mouse according to the invention contains a single copy of the transgene encoding a constitutively active form of the IKK2.

The terms “a single copy” or “low copy” as used herein are in reference to the number of identical copies of a transgene that are present in a transgenic event. An event with a single copy number or low copy has only one complete copy of the IKK2 transgene or introduced polynucleotide of interest incorporated into its genome, with one and only one copy of each transcriptional cassette comprising the IKK″ gene or introduced polynucleotide of interest and no partial copies. The expression “single copy” or “low copy” will be used throughout the present application in relation to other transgenes.

In a preferred embodiment, the transgene encoding a constitutively active form of the IKK2 protein is under the operative control of the Rosa26 promoter.

The term “Rosa26” or “Rosa26 promoter” refers to the murine promoter described in Zambrowicz et al., Proc. Nat. Acad. Sci. 94:3789-94(1997), and functional portions thereof. Functional portions refers to a fragment or the Rosa26 promoter that drives expression of an operably linked nucleic acid to a level that is at least about 75%, 80%, 85%, 90%, 95%, or 100% of the level of expression that would result if the nucleic acid were operably linked to a Rosa26 promoter. The term “Rosa26 promoter” will be used throughout the present document with the same meaning in relation to a series of different proteins or transgenes.

“Operably linked” means that a gene and a regulatory sequence(s) are connected in such a way as to permit gene expression when the appropriate molecules (e.g., transcriptional activator proteins) are bound to the regulatory sequence(s).

In another embodiment, the MYC protein is the human MYC protein. The term “human MYC protein” refers to the protein encoded by the gene shown in the Ensembl database under accession number ENSG00000136997 (Ensemble release 107, July 2022).

In another embodiment, the MYC protein is under the control of the CAG promoter.

The term “CAG promoter” refers herein to a promoter that is a fusion promoter and generally contains the Cytomegalovirus (CMV) Immediate Early Enhancer, two parts of the chicken beta-actin promoter and part of the rabbit beta1-globin promoter (see FIG. 1). More specifically, in the 1132 bp long promoter constructs, nucleotide region 15-380 corresponds to the CMV Immediate Early Enhancer, nucleotide regions 381-861 and 861-1014 correspond to the two parts of the chicken beta-actin promoter sequences, and nucleotide region 1023-1126 represents the rabbit beta1-globin promoter sequence. Other short sequences on the construct represent linker sequences (Niwa, H et al., Gene 1991, 108:193).

In a preferred embodiment the conditionally activatable transgene encoding the MYC protein is inserted in the Rosa26 locus.

In another particular embodiment, the genetically engineered mouse of the invention comprises a single copy of the conditionally activatable transgene encoding the MYC protein.

In another embodiment, the conditionally activatable transgene encoding the K-Ras^{G12D} protein is located in the endogenous Kras locus replacing the wild-type Kras gene and under the operative control of the Kras promoter.

The core promoter region of kras is encompassed within the region from +50 bp through −510 bp, in relation to the transcriptonal start site (TSS). The DNA within this region is highly G/C-rich (˜75%), putatively capable of forming higher order non-B-DNA structures, and contains two nuclease hypersensitivity elements.

In a preferred embodiment, the genetically engineered mouse contains a single copy of the conditionally activatable transgene encoding the K-Ras^{G12D} protein.

In another embodiment, the genetically engineered mouse contains a single copy of the of the conditionally inactivatable Trp53 gene.

In a preferred embodiment, the conditionally inactivatable Trp53 gene is replacing the endogenous Trp53 gene, wherein the conditionally inactivatable Trp53 gene comprises recombinase target sites flanking a region in the gene which is required for the expression of a functional Trp53 protein and wherein conditionally inactivatable Trp53 gene is found under the operative control of the Trp53 promoter.

The term “recombinase target site” or “recombinase recognition site (RRS)” is used according to the definitions provided in the art. Thus, it refers to a short nucleic acid site or sequence, that is recognized by a site-specific recombinase and which becomes the crossover region during a site-specific recombination event. Non-limiting examples of recombinase recognition site include lox sites, att sites and frt sites. The term “lox site” as used herein refers to a nucleotide sequence at which the product of the cre gene of bacteriophage P1, the Cre recombinase, can catalyze a site-specific recombination event. A variety of lox sites are known in the art, including the naturally occurring loxP, loxB, loxL and loxR, as well as a number of mutant, or variant, lox sites, such as loxP511, lox5171, loxP514, loxA86, loxA117, loxC2, loxP2, loxP3 and lox P23. The term “frt site” as used herein refers to a nucleotide sequence at which the product of the flp gene of the yeast 2 micron plasmid, FLP recombinase, can catalyze site-specific recombination. Frt sites include the naturally occurring “FRT” as well as the “F3” and “F5” site. The orientation of the recombinase recognition sites dictates one of three types of site specific recombination reactions: (i) excision, if two identical recombinase recognition sites are present in the same direction; (ii) inversion, if two identical recombinase recognition site are present in opposite direction and (iii) exchange, if two different (heterotypic) recombinase recognition sites are present in opposite or identical direction.

Also the term “recombinase” is used in accordance with the definitions provided in the art. Thus, it refers to a genetic recombination enzyme that mediates site-specific recombination in cells. Site specific recombinases are naturally occurring only in prokaryotes and lower eukaryotes. There are two classes of site-specific recombinases, tyrosine recombinases (integrases) and serine recombinases (invertases/resolvases) . . . . Non-limiting examples of tyrosine recombinases include Cre recombinase from E. coli phage P1, FLP recombinase from yeast 2m episome, I integrase from E. coli I phage and XerC/XerD recombinase from E. coli. Non-limiting examples include Hin recombinase from Salmonella flagella antigen switch, gamma-delta resolvase from the Tn 1000 transposon and ΦC31 integrase from Streptomyces phage (large serine subclass).

The requirement that a recombinase recognition site is specifically recognised by a particular recombinase (such as for example the first, second, third and/or fourth recombinase) means that said recombinase recognition site is only recognised by said particular recombinase. For example, a loxP site is only recognised by Cre recombinase but not by FLP recombinase. Thus, a loxP recombinase recognition site is specifically recognised by Cre recombinase. While one recombinase recognition site can only be recognised by one recombinase it is nonetheless possible that one recombinase can recognise multiple recombinase recognition sites. For example, Cre recombinase not only recognises loxP, but also the above recited recombinase recognition sites loxB, loxL, loxR, loxP511, loxP514, loxA86, loxA117, loxC2, loxP2, loxP3 and lox P23. As site-specific recombination only occurs between matching recombinase recognition sites, the use of different recombinase recognition sites within the transgene cassette ensures that recombination only occurs between the genome and the cassette but not within the cassette.

Thus, in the present case, as mentioned above, the conditional Trp53 gene deletion is achieved by preparing a mouse model in which either the whole gene of interest or a critical gene segment is flanked by two recombinase target sites. In an embodiment, the recombinase target sites are two loxP sites. In another embodiment, the recombinase target sites are inserted into a wild-type mouse. In another embodiment, the conditional mouse lines containing two loxP sites are usually designed to be wild-type mice. The mouse model is crossed with transgenic strain expressing the corresponding recombinase in one or several cell types, thus, wherein the conditional mutant is generated by crossing these two strains such that target gene inactivation occurs in a spatial and temporal restricted manner, according to the pattern of recombinase expression in the second transgenic strain. Alternatively, the recombinase may be inserted and therefore expressed in the same mouse harbouring the RRS flanked gene segment.

In a preferred embodiment, the conditionally inactivatable Trp53 gene is characterized in that it comprises recombinase target sites which flank exons 2 to 10 of the Trp53 gene.

In another embodiment, the transgene encoding the BCL2 protein is under the control of the Eμ immunoglobulin heavy chain enhancer and the SV40 promoter.

The term “enhancer” is used according to its art-recognized meaning. It is intended to mean a sequence found in eukaryotes and certain eukaryotic viruses, which can increase transcription from a gene when located (in either orientation) up to several kilobases from the gene being studied. These sequences usually act as enhancers when on the 5′ side (upstream) of the gene in question. However, some enhancers are active when placed on the 3′ side (downstream) of the gene. In some cases, enhancer elements can activate transcription from a gene with no (known) promoter.

The Eμ enhancer is a major control element in the IgH locus located downstream of the J_{H} segments. It is an intronic region of DNA (40 to 1500 bp in length) within the 700-bp intron between the J heavy chain segment and the C mu(μ) segment of the immunoglobulin heavy chain gene locus. It can bind an activator protein to increase or activate transcription of the heavy chain gene. It is capable of directing expression in B cells of the mouse wherein the enhancer sequence is operably linked to a nucleic acid encoding the protein of interest. In a particular embodiment, the Eμ enhancer is the one disclosed in S D Gillies, et al., Cell. 1983 July; 33(3):717-28 or in J Banerji, et al., Cell. 1983 July; 33(3):729-40.

The SV40 promoter, also called simian vacuolating virus 40 promoter or simian virus 40 promoter, contains three elements, i) the TATA box, located approximately 20 base-pairs upstream from the transcriptional start site; ii) 21 base-pair repeats containing six GC boxes which is the site that determines the direction of transcription; and iii) 72 base-pair repeats which are transcriptional enhancers.

In a particular embodiment, the complete sequence of the SV40 promoter is the one disclosed in W Fiers, et al., Nature. 273(5658):113-20(1978) or in V B Reddy, Science. 200(4341):494-502(1978). In another embodiment, the promoter region in SV40 is as one disclosed in C Benoist & P Chambon, Nature 290, 304-310(1981) or in B J Byrne, Proc Natl Acad Sci USA. 80(3):721-725(1983). In another embodiment, the enhancer region in SV40 is the one disclosed in Banerji J, et al., Cell 27:299-308(1981) or in Moreau P, et al., Nucl Acids Res 9:6047-6068(1981).

In another embodiment, the transgene encoding the MAF protein is under the operative control of the Eμ immunoglobulin heavy chain enhancer and the V_{H} promoter.

In the context of the present specification, the term “V_{H} promoter” refers to the promoter of the “V_{H} gene”. The “V_{H} gene” refers to the gene encoding the immunoglobulin heavy chain variable region (V_{H}).

In another embodiment, the transgene encoding the Cyclin-D1 protein is under the operative control of the Eμ immunoglobulin heavy chain enhancer and the V_{H} promoter.

In another embodiment, the conditionally activatable transgene encoding the MMSET-II protein is under the operative control of the CAG promoter.

In a preferred embodiment, the conditionally activatable transgene encoding the MMSET-II protein is inserted in the Rosa26 locus.

In another embodiment, the genetically engineered mouse contains a single copy of the conditionally activatable transgene encoding the MMSET-II protein.

In another embodiment, the one or more transgenes that are conditionally activatable comprise a region comprising a transcription terminator site flanked by recombinase target sites, said region being located at a position which causes premature termination of the transcription.

As used herein, the phrase “transcription termination site” or “transcription termination sequence” refers to a nucleic acid sequence (or base) that marks the end of transcription of a gene. It will be appreciated that the transcription termination site may mark the end of a gene, or may be a premature site present in the 5′ UTR of a gene (i.e. a premature transcription termination site).

Examples or transcription termination sites include, without limitation, the cleavage of mRNA precursor in a site-specific manner in the 3′-untranslated region, followed by polyadenylation of the upstream cleavage product dependent on dependent on CPF, CFIA and CFIB complexes; the transcription termination at genes encoding snRNAs and snoRNAs by the Nrd1-Nab3-Sen1 (NNS) complex or the Rnt1 endonuclease; or the transcription termination associated with Pol II pausing.

In a particular embodiment, the transcription termination sequence comprises a polyadenylation signal, referred to as polyadenylation/termination sequence. In another embodiment, the termination sequence is derived from SV40 virus.

In a particular embodiment, one or more of the conditionally activatable transgenes further comprise a reporter gene that is co-expressed upon activation of the transgene

By the term “reporter gene(s),” it is used herein in a broad sense and is meant to define a gene which encodes a polypeptide, whose expression can be detected in a variety of known assays and wherein the level of the detected signal indicates the presence of said report. Such genes include, without limitation, green fluorescent protein (GFP), glucuronidase (GUS), luciferase, chloramphenicol transacetylase (CAT), and β-galactosidase.

In a preferred embodiment, the reporter gene that is co-expressed upon activation of the transgene is a gene encoding a fluorescent protein or the human CD2 gene.

The human CD2 gene, as used herein, refers to a surface antigen found on all peripheral blood T-cells and which can be used as a surface marker using antibodies which are capable of binding to the extracellular domain. In one embodiment, the CD2 marker is as disclosed in Sander S, et al., Cancer Cell. 2012 Aug. 14; 22(2):167-79.

In another particular embodiment, the conditionally activatable transgene and the reporter gene are found within the same gene construct under the control of the same promoter and an IRES is inserted between the conditionally activatable transgene and the reporter gene.

IRES stands for a nucleic acid sequence encoding an “internal ribosome entry site” sequence. IRES sequences are often used in molecular biology to co-express several genes under the control of the same promoter, thereby mimicking a polycistronic mRNA. IRES is short for internal ribosome entry site, which is a nucleotide sequence that allows for translation initiation in the middle of a messenger RNA (mRNA) sequence as part of the greater process of protein synthesis. Usually, in eukaryotes, translation can only be initiated at the 5′ end of the mRNA molecule, since 5′ cap recognition is required for the assembly of the initiation complex. IRES mimics the 5′ cap structure, and is recognized by the 4OS pre-initiation complex. When an IRES segment is located between two reporter open reading frames in a eukaryotic mRNA molecule (a bicistronic mRNA), it can drive translation of the downstream protein coding region independently of the 5′-cap structure bound to the 5′ end of the mRNA molecule. In such a setup both proteins are produced in the cell. The first protein located in the first cistron is synthesized by the cap-dependent initiation approach while translation initiation of the second protein is directed by the IRES segment located in the intercistronic spacer region between the two reporter protein coding regions.

In some embodiments, the IRES element is placed at the 5′UTR sequences of the corresponding two genes to be translated.

In another embodiment, the genetically engineered mouse according to the invention, additionally comprises within its genome a sequence encoding for a recombinase which is specific for the recombinase target sites flanking the transcription terminator sequence within the conditionally activatable transgene or transgenes and/or specific for the recombinase target sites present in the Trp53 gene region which is required for the expression of a functional Trp53 protein.

In a particular embodiment, the recombinase is the Cre recombinase and the recombinase target sites are loxP sites.

Cre recombinase is a 38-kDa integrase encoded by bacteriophage P1 and mediates site-specific recombination between 34-bp sequences referred to as loxP (locus of crossover (x) in P1 bacteriophage) sites (reviewed in Sauer, 1998; Nagy, 2000). A loxP site is composed of a nonpalindromic 8-bp sequence (GCATACAT or ATGTATGC) flanked on either side by 13-bp inverted repeats (ATAACTTCGTATA; Hamilton and Abremski, 1984; Hoess et al., 1982) (SEQ ID NO: 44). Cre-based recombination between the two loxP sites leads to a reciprocal exchange of DNA strands. Cre-mediated recombination requires a minimum of 82 bp between two loxP sites for efficient recombination, though there is no upper limit

Other similar systems such as FLP-FRT may be used in genome engineering in ES cells and transgenic mice (Dymecki et al., 1996a,b). FLP is a 423-amino acid monomeric peptide encoded within the 2-μm yeast plasmid of Saccharomyces cerevisiae that uses phosphotyrosine for energy, whereas FRT is composed of an 8-bp asymmetric spacer (TCTAGAAA or TTTCTAGA) surrounded by 13-bp repeats (GAAGTTCCTATTC (SEQ ID NO: 45)). The asymmetric region dictates whether excision or inversion occurs after recombination. Both Cre and FLP recombinases belong to the tyrosine site-specific recombinase class; thus, they act similarly. However, FLP-mediated gene deletion is less inefficient. Therefore, an enhanced form of FLP, FLPe, has been developed, which makes the FLPe-FRT system an alternative to the Cre-loxP system (Rodriguez et al., 2000). also, the Cre-loxP and FLPe-FRT systems may be combined for preparing targeting constructs in such a way that FLPe-FRT system is responsible for removing the selection marker, whereas the Cre-loxP system takes care of the DNA fragment under study. The International Knockout Mouse Consortium (IKMC) often utilizes such a combined strategy for generating new mouse lines.

Besides the Cre-loxP and FLPe-FRT systems, Dre-rox, a related recombinase-DNA pair, has been reported to work in mice (Anastassiadis et al., 2009). Cre mutants and chimeric Cre/FLP may offer another alternative strategy (Hartung and Kisters-Woike, 1998; Shaikh and Sadowski, 2000). In addition, besides inducing recombination between two loxP sites, Cre can induce specific recombination between two lox511 sites; the lox511 site is an alternative recognition site for Cre that has a different spacer sequence compared to the loxP site (Soukharev et al., 1999). Since recombination between loxP and lox511 is very inefficient, the combination of loxP and lox511 pairs has been used for site-specific gene insertion. There are other integrases, such as phiC31 and phiBT1, that belong to the serine site-specific recombinase class and induce directional rather than reversible recombination. These have been reported to function in eukaryotic genome engineering, such as in yeast, Drosophila, and mammalian cells (Thyagarajan et al., 2001; Groth et al., 2004; Keravala and Calos, 2008; Xu et al., 2008).

In another embodiment, the sequence encoding the recombinase is placed under the operative control of a promoter specific of B lymphocytes, for immature pre-B or mature germinal center (GC) B lymphocytes; preferably a promoter specific for mature GC B lymphocytes. In a particular embodiment the promoter for pre-B is the mb1-cre. In another embodiment, the promoter of the GC B lymphocytes is cgamma1-cre.

In a preferred embodiment, the placing of the recombinase coding sequence under the control of a promoter specific of B lymphocytes is achieved by inserting the recombinase coding sequence into the cγ1 locus or into the mb1 locus, or wherein the recombinase coding sequence is under the control of the Cgamma1 protein (Cγ1-cre) promoter or of the Cd79a protein (mb1-cre) promoter. Examples of Mb1-cre and cgamma1-cre promoters can be found in Hobeika E, et al., Proc Natl Acad Sci USA. 2006 Sep. 12; 103(37):13789-94 and in Casola S, et al., Proc Natl Acad Sci USA. 2006 May 9; 103(19):7396-401 respectively.

The expression “cγ1” refers to the Ig γ1 constant region gene segment.

The expression “Cd79a” also known as B lymphocyte-specific MB1 Protein, B Cell antigen receptor complex-Associated Protein alpha chain, CD79a Molecule immunoglobulin Associated alpha, Ig-alpha, IGA, IgM-alpha, Immunoglobulin-Associated alpha, Ly54, MB-1 Membrane GlycoProtein or Membrane-Bound Immunoglobulin-Associated Protein

In a preferred embodiment, the genetically engineered mouse contains a single copy of the gene encoding the recombinase.

In another particular embodiment, the genetically engineered mouse according to the invention is selected from the group consisting of:
(i) a genetically engineered mouse that comprises a conditionally activatable transgene encoding a constitutively active form of the IKK2 protein and a transgene encoding the human BCL2 protein,
(ii) a genetically engineered mouse that comprises a conditionally activatable transgene encoding a constitutively active form of the IKK2 protein, a transgene encoding the human BCL2 protein and a conditionally activatable transgene encoding the K-Ras^{G12D} protein,
(iii) a genetically engineered mouse that comprises a conditionally activatable transgene encoding a constitutively active form of the IKK2 protein, a transgene encoding the human BCL2 protein and a transgene comprising a conditionally inactivatable sequence encoding the p53 polypeptide,
(iv) a genetically engineered mouse that comprises a conditionally activatable transgene encoding a constitutively active form of the IKK2 protein, a transgene encoding the human BCL2 protein, a transgene comprising a conditionally inactivatable sequence encoding the p53 polypeptide and a conditionally activatable transgene encoding the K-Ras^{G12D} protein,
(v) a genetically engineered mouse that comprises a conditionally activatable transgene encoding a constitutively active form of the IKK2 protein, a transgene encoding the human BCL2 protein and a transgene encoding the Cyclin-D1 protein,
(vi) a genetically engineered mouse that comprises conditionally activatable transgene encoding a constitutively active form of the IKK2 protein, a transgene encoding the human BCL2 protein and a transgene encoding the MAF protein.
(vii) a genetically engineered mouse that comprises a conditionally activatable transgene encoding a constitutively active form of the IKK2, a transgene encoding the human BCL2 protein and a conditionally activatable transgene encoding the MMSET-II protein
(viii) a genetically engineered mouse that comprises conditionally activatable transgene encoding a constitutively active form of the IKK2 protein and a transgene encoding the MYC protein.
(ix) a genetically engineered mouse that comprises conditionally activatable transgene encoding a constitutively active form of the IKK2 protein, a transgene encoding the MYC protein and a conditionally activatable transgene encoding the K-Ras^{G12D} protein,
(x) a genetically engineered mouse that comprises conditionally activatable transgene encoding a constitutively active form of the IKK2 protein, a transgene encoding the MYC protein and a transgene comprising a conditionally inactivatable sequence encoding the p53 polypeptide,
(xi) a genetically engineered mouse that comprises conditionally activatable transgene encoding a constitutively active form of the IKK2 protein, a transgene encoding the MYC protein and a transgene encoding the human BCL2 protein,
(xii) a genetically engineered mouse that comprises conditionally activatable transgene encoding a constitutively active form of the IKK2 protein, a transgene encoding the MYC protein and a transgene encoding the MAF protein,
(xiii) a genetically engineered mouse that comprises conditionally activatable transgene encoding a constitutively active form of the IKK2 protein and a conditionally activatable transgene encoding the MMSET-II protein, and
(xiv) a genetically engineered mouse that comprises a transgene encoding the MYC protein and conditionally activatable transgene encoding the MMSET-II protein.

In another preferred embodiment, the genetically engineered mouse of items (i) to (xiv) additionally comprise within its genome a sequence encoding for a recombinase which is specific for the recombinase target sites flanking the transcription terminator sequence within the conditionally activatable transgene.

In another embodiment, the sequence encoding the recombinase is placed under the operative control of a promoter specific for immature pre-B cells or mature germinal center (GC) B lymphocytes. In a still more preferred embodiment, the sequence encoding for a recombinase is under operative control of the Cgamma1 protein (Cγ1-cre) promoter or under the control of the Cd79a gene promoter.

In another embodiment, the genetically engineered mouse according to any of the preceding aspects or particulars, further comprises a transgene encoding a human or humanized protein which confers sensitivity to an anti-cell blood cancer therapy, said transgene being inserted in the locus of the mouse orthologue.

A “humanized” protein is typically a chimeric mammalian-type protein which is partially comprised of a human-derived protein sequence.

In a particular embodiment, genetically engineered mouse comprising the transgene encoding a human or humanized protein is heterozygous or homozygous for said transgene.

In another embodiment, the protein which confers sensitivity to an anti-cell blood cancer therapy is Crbn^{I139V}.

The protein CRBN, also known as protein Cereblon, is a 442-amino acid protein with multifunction, locates in the cytoplasm, nucleus, and peripheral membrane of the human brain and other tissues. The 139V Crbn is known to confer sensitivity to thalidomide and its derivatives, such as lenalidomide and pomalidomide.

In another embodiment, the protein which confers sensitivity to an anti-cell blood cancer therapy is a protein which is expressed in the surface of malignant plasma cells or in the surface of cells of the immune system. For instance, in tumors derived from B-lymphocytes such as chronic lymphocytic leukemia, diffuse large B-cell lymphoma, or lymphoplasmacytic lymphoma, therapies targeting surface receptor CD20 are effective by having an effect against the protein expressed by the malignant cells; this anti-CD20 therapy (i.e. Rituximab) has been approved to treat patients with these malignancies in combination with chemotherapy (i.e. CHOP-cyclophosphamide, adriamycin-hydroxydaunorubicin, vincristine-oncovin and prednisone). Similarly, in multiple myeloma, antibodies targeting the CD38 surface receptor (i.e. Daratumumab) are in clinical use in combination with chemo-immunotherapy (i.e. VRD, bortezomib-velcade, lenalidomide-revlimid and dexamethasone), as these inhibit CD38 and downstream signaling in myeloma cells that leads to tumor cell death.

In a particular embodiment, the protein which is expressed in the surface of malignant plasma cells is BCMA, SLAMF7, CD38, FcFR5 or GPRC5D or the protein which is expressed in the surface of the cells of the immune system is CD3, CD28 or CD137. In another embodiment, the protein which confers sensitivity to an anti-cell blood cancer therapy is an immune checkpoint and the anti-cell blood cancer therapy is a therapy based on an immune checkpoint inhibitor.

The term “checkpoint inhibitor”, as used herein, relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint or checkpoint regulators. They act as molecular “gatekeepers” that allow extracellular information to dictate whether cell cycle progression and other intracellular signaling processes should proceed.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR, and various B7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-HI; MEDI4736), MK-3475 (PD-1 blocker), Nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PDL1 antibody), and ipilimumab (anti-CTLA-4 checkpoint inhibitor). Checkpoint protein ligands include, but are not limited to PD-LI, PD-L2, B7-H3, B7-H4, CD28, CD86 and TIM-3.

In a particular embodiment, the immune checkpoint is 4-1BB, PD-1 and PD-L1.

The protein 4-1BB, also known as Tumor Necrosis Factor Receptor Superfamily Member 9 (TNFRSF9) is a member of the TNF-receptor superfamily. This receptor contributes to the clonal expansion, survival, and development of T cells. It can also induce proliferation in peripheral monocytes, enhance T cell apoptosis induced by TCR/CD3 triggered activation, and regulate CD28 co-stimulation to promote Th1 cell responses. The expression of this receptor is induced by lymphocyte activation.

Programmed cell death protein 1, also known as PD-1 and CD279 (cluster of differentiation 279), is a protein on the surface of T and B cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells.

PD-L1, also known as Programmed Cell Death 1 Ligand 1 is an immune inhibitory receptor ligand that is expressed by hematopoietic and non-hematopoietic cells, such as T cells and B cells and various types of tumor cells. This protein is a type I transmembrane protein that has immunoglobulin V-like and C-like domains. Interaction of this ligand with its receptor inhibits T-cell activation and cytokine production. During infection or inflammation of normal tissue, this interaction is important for preventing autoimmunity by maintaining homeostasis of the immune response. In tumor microenvironments, this interaction provides an immune escape for tumor cells through cytotoxic T-cell inactivation. Alternative splicing of the gene encoding the PD-L1 protein results in multiple transcript variants.

Non-limiting examples of “rodents” are mice, rats, squirrels, chipmunks, gophers, porcupines, beavers, hamsters, gerbils, guinea pigs, degus, chinchillas, prairie dogs, and groundhogs. Preferably, the rodents are selected from the group consisting of mice and rats.

### Bicistronic Gene Constructs Suitable for the Generation of Mouse Models of Human Myeloma and Mouse Models Obtained Using the Bicistronic Gene Constructs

In a third aspect, the invention relates to a bicistronic gene construct selected from the group consisting of:
i) a bicistronic gene construct comprising a first cistron which is conditionally activatable and which encodes a constitutively active form of the IKK2 protein and a second cistron that encodes the BCL2 protein,
ii) a bicistronic gene construct comprising a first cistron which is conditionally activatable and that encodes a constitutively active form of the IKK2 protein and a second cistron which is conditionally activatable and that encodes the MYC protein,
iii) a bicistronic gene construct comprising a first cistron which is conditionally activatable and that encodes a constitutively active form of the IKK2 protein and a second cistron which is conditionally activatable and that encodes the MMSET-II protein, and
iv) a bicistronic gene construct comprising a first cistron which is conditionally activatable and that encodes the MYC protein and a second cistron which is conditionally activatable and that encodes the MMSET-II protein; and
wherein the gene construct is under operative control of a promoter and wherein an internal ribosomal entry site is present between the first and second cistrons.

The term “bicistronic gene,” is typically defined as a gene capable of providing a RNA molecule that encodes two proteins/polypeptides.

The term “cistron” is herein used as alternative term for “gene”.

Preferably, the protein expression unit comprises a multicistronic gene. Units comprising two cistrons can be transcribed as a single mRNA. Translation of the second coding regions present on that RNA can be achieved in various ways, including the use of translation reinitiation sites or internal ribosome entry sites (IRES), the latter of which is preferred. Advantages of bi-cistronic units are plurifold and include easy selection of clones expressing a protein of interest, for instance, by placing the nucleic acid encoding a dominant selectable marker protein downstream of nucleic acid encoding a protein or polypeptide of interest. Any type of promoter may be used in the present invention as long as it is operable for allowing transcription in the protein expression unit at a certain time point, or continuously.

As mentioned above, internal ribosome entry sites (IRES) are cis-acting RNA regions that promote internal initiation of protein synthesis by recruiting the 40S ribosomal subunit through cap-independent mechanisms. IRES elements are generally located within the 5′ UTR, although a few examples of viral and cellular IRES elements placed within the coding gene sequence have been described. Within the context of the present invention, different IRES are placed at 5′ UTR sequences of the corresponding two genes to be translated.

In a particular embodiment, the constitutively active form of the IKK2 protein contains the S177E and S181E mutations.

In another embodiment, the cistron encoding a constitutively active form of the IKK2 protein is under the operative control of the Rosa26 promoter.

In another embodiment, cistron encoding the BCL2 protein is under the control of the Eμ immunoglobulin heavy chain enhancer and the SV40 promoter.

In another embodiment, the MYC protein is the human MYC protein.

In a preferred embodiment, the conditionally activatable cistron encoding the MYC protein is under the control of the CAG promoter.

In another embodiment, wherein the conditionally activatable cistron encoding the MMSET-II protein is under the operative control of the CAG promoter.

In a particular embodiment, the construct is adapted to be inserted into the Rosa26 locus or into the Hprt locus.

The Hprt protein refers to the Mus musculus hypoxanthine guanine phosphoribosyl transferase (Hprt), which mRNA is disclosed in RefSeq NM_013556). This protein catalyzes conversion of hypoxanthine to inosine monophosphate and guanine to guanosine monophosphate via transfer of the 5-phosphoribosyl group from 5-phosphoribosyl 1-pyrophosphate. This enzyme plays a central role in the generation of purine nucleotides through the purine salvage pathway. Reference to the Hprt locus can be is provided by RefSeq, September 2015, Gencode Transcript: ENSMUST00000026723.9 and Gencode Gene: ENSMUSG00000025630.9. Transcript (Including UTRs) Position: mm39 chrX: 52,077,014-52,110,536 Size: 33,523 Total Exon Count: 9 Strand: +. Coding Region Position: mm39 chrX: 52,077,101-52, 109,991 Size: 32,891 Coding Exon Count: 9.

In another preferred embodiment, the cistrons which are conditionally activatable comprise a transcription terminator sequence flanked by recombinase target sites between the promoter and the transcriptional start site of the first cistron.

In a fourth aspect, the invention relates to a vector comprising the bicistronic construct according to the invention.

By “vector” is meant a nucleic acid molecule, preferably a DNA molecule derived, for example, from a plasmid, bacteriophage or virus, into which a nucleic acid sequence may be inserted or cloned. Non-limiting examples of vectors include plasmids, phages, cosmids, phagemids, yeast artificial chromosomes (YAC), bacterial artificial chromosomes (BAC), human artificial chromosomes (HAC), viral vectors such as adenoviral vectors or retroviral vectors, and other DNA sequences which are conventionally used in genetic engineering and/or able to convey a desired DNA sequence to a desired location within a host cell.

A vector preferably contains one or more restriction sites and may be capable of autonomous replication in a defined host cell including a target cell or tissue or a progenitor cell or tissue thereof, or be partially or entirely integrable with the genome of the defined host such that the cloned sequence is reproducible. Accordingly, the vector may be an autonomously replicating vector, i.e. a vector that exists as an extrachromosomal entity, the replication of which is independent of chromosomal replication, e.g. a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome/s into which it has been integrated. The choice of the vector will typically depend on the compatibility of the vector with the host cell into which the vector is to be introduced.

The vector may further comprise one or more nucleic acid sequences encoding selectable marker such as auxotrophic markers (e.g., LEU2, URA3, TRP 1 or HIS3), detectable labels such as fluorescent or luminescent proteins (e.g., GFP, eGFP, DsRed, CFP), or protein conferring resistance to a chemical/toxic compound (e.g., MGMT gene conferring resistance to temozolomide). These markers can be used to select or detect host cells comprising the vector and can be easily chosen by the skilled person according to the host cell.

The vector of the invention is preferably a viral genome vector including any element required to establish the expression of the recombinant nucleic acid molecule of the invention in a host cell such as, for example, a promoter, an ITR, a ribosome binding element, terminator, enhancer, selection marker, intron, polyA signal, and/or origin of replication.

In some embodiments, the vector is a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

In particular embodiments, the vector is a retroviral vector, preferably a lentiviral vector or a non-pathogenic parvovirus.

As known in the art, depending on the specific viral vector considered for use, suitable sequences should be introduced in the vector of the invention for obtaining a functional viral vector, such as AAV ITRs for an AAV vector, or LTRs for lentiviral vectors.

The recombinant nucleic acid molecule or expression cassette of the invention may be introduced into the vector by any method known by the skilled person.

All the embodiments of the recombinant nucleic acid molecule and expression cassette of the invention are also contemplated in this aspect.

The vector of the invention may be packaged into a virus capsid to generate a “viral particle”. Thus, in a further aspect, the present invention also relates to a viral particle comprising a vector of the invention.

All the embodiments of the recombinant nucleic acid molecule, the expression cassette or the vector of the invention are also contemplated in this aspect.

Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e. g., translation initiation codon, promoters, enhancers, and/or insulators), T2A, P2A or similar sequences (Smyczak et al., 2004), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, the nucleic acid molecules of the invention are operatively linked to such expression control sequences allowing expression in cells.

Possible examples for regulatory elements ensuring the initiation of transcription comprise the cytomegalovirus (CMV) promoter, SV40-promoter, RSV-promoter (Rous sarcome virus), the lacZ promoter, the gai10 promoter, human elongation factor 1a-promoter, CMV enhancer, CaM-kinase promoter, the Autographa californica multiple nuclear polyhedrosis virus (AcMNPV) polyhedral promoter or the SV40-enhancer. Examples for further regulatory elements in prokaryotes and eukaryotic cells comprise transcription termination signals, such as SV40-poly-A site or the tk-poly-A site or the SV40, lacZ and AcMNPV polyhedral polyadenylation signals, downstream of the polynucleotide.

An expression vector according to this invention is capable of directing the replication, and the expression, of the nucleic acid molecule and encoded enzyme. Suitable expression vectors which comprise the described regulatory elements are known in the art such as Okayama-Berg cDNA expression vector pcDV1 (Pharmacia), pRc/CMV, pcDNA1, pcDNA3 (In-Vitrogene, as used, inter alia in the appended examples), pSPORT1 (GIBCO BRL) or pGEMHE (Promega), or prokaryotic expression vectors, such as lambda gt11, pJOE, the pBBR1-MCS-series, pJB861, pBSMuL, pBC2, pUCPKS, pTACT1 or, preferably, the pET vector (Novagen).

In a fifth aspect, the invention relates to a genetically engineered mouse comprising a bicistronic gene construct of the third aspect of the invention or a vector of the fourth aspect of the invention integrated in its genome.

As mentioned above, mice comprising the bi-cistronic construct or vector of the invention can be produced by any suitable method known in the art, such as manipulation of embryos, embryonic stem cells, etc. Genetically engineered mice may be made through homologous recombination, where the endogenous locus is altered. Alternatively, a nucleic acid construct is randomly integrated into the genome. Vectors for stable integration include plasmids, retroviruses and other animal viruses, YACs, and the like.

Numerous methods for preparing transgenic mice are now known and others will likely be developed. See, e.g., U.S. Pat. Nos. 6,252,131, 6,455,757, 6,028,245, and 5,766,879. Any method that produces a transgenic mouse is suitable for use in the practice of the present invention. The microinjection technique described is particularly useful for incorporating transgenes into the genome without the accompanying removal of other genes.

In particular, the method of generating a transgenic organism may comprise:
introducing an bi-cistronic cassette or a vector of the invention in a non-human embryonic stem cell; —obtaining a transgenic embryonic stem cell wherein the recombinant nucleic acid molecule of the invention is inserted into the genome, preferably by homologous recombination;
injecting said transgenic embryonic stem cell into a blastocyst of a mouse to form chimeras; and
reimplanting said injected blastocyst into a foster mother.

Embryonic stem (ES) cell are typically obtained from pre-implantation embryos cultured in vitro. Preferably, the cassette or vector of the invention is transfected into said ES cell by electroporation. The ES cells are cultured and prepared for transfection using methods known in the related art. The ES cells that will be transfected with the cassette or vector of the invention are derived from embryo or blastocyst of the same species as the developing embryo or blastocyst into which they are to be introduced. ES cells are typically selected for their ability to integrate into the inner cell mass and contribute to the germ line of an individual when introduced into the mouse in an embryo at the blastocyst stage of development. In one embodiment, the ES cells are isolated from the mouse blastocysts.

After transfection into the ES cells, the recombinant nucleic acid molecule of the invention integrates with the genomic DNA of the cell in order to produce an antibody of the invention as defined below.

After transfection, the ES cells are cultured under suitable condition to detect transfected cells. For example, when the cassette or vector comprises a marker gene, e.g. an antibiotic resistant marker, e.g. neomycin resistant gene, the cells are cultured in that antibiotic. The DNA and/or protein expression of the surviving ES cells may be analyzed using Southern Blot technology in order to verify the proper integration of the cassette.

The selected ES cells are then injected into a blastocyst of a mouse to form chimeras. The mouse is preferably a mouse, a hamster, a rat or a rabbit. More preferably, the mouse is a mouse.

In particular, the ES cells may be inserted into an early embryo using microinjection. The injected blastocysts are re-implanted into a foster mother. When the progenies are born, they are screened for the presence of the recombinant nucleic acid molecule, expression cassette or vector of the invention, e.g. using Southern Blot and/or PCR technique. The heterozygotes are identified and are then crossed with each other to generate homozygous mice.

In another embodiment, the method of generating a transgenic organism may comprise:
introducing in a non-human fertilized egg (i) an expression cassette or vector of the invention and (ii) a nuclease system used to target the cassette or vector at the correct locus by homologous recombination;
obtaining a transgenic fertilized egg wherein the bicistronic cassette or vector of the invention is inserted into the genome by homologous recombination; and
reimplanting said injected fertilized egg into a foster mother.

The nuclease system used to target the cassette or vector at the correct locus may be any suitable system known by the skilled person, such as systems involving ZFN, TALE or CRISPR/Cas9 nucleases

Preferably, the nuclease system is a CRISPR/Cas9 system. To use Cas 9 to modify genomic sequences, the protein can be delivered directly to a cell. Alternatively, an mRNA that encodes Cas 9 can be delivered to a cell, or a gene that provides for expression of an mRNA that encodes Cas 9 can be delivered to a cell. In addition, either target specific crRNA and a tracrRNA or target specific gRNA(s) can be delivered to the cell (these RNAs can alternatively be produced by a gene constructed to express these RNAs). Selection of target sites and designed of crRNA/gRNA are well known in the art.

In a preferred embodiment, the engineered mouse comprising a bicistronic gene construct or a vector according to the invention integrated in its genome further comprises within its genome a sequence encoding for a recombinase which is specific for the recombinase target sites flanking the transcription terminator sequence within the conditionally activatable cistron. Recombinase target sites suitable for their use in the engineered mouse comprising a bicistronic gene construct or a vector according to the invention are as explained above in the context of the first and second aspects of the invention.

In some embodiments, recombinase is the Cre recombinase and the recombinase target sites are loxP sites. In yet another embodiment, the sequence encoding the recombinase is placed under the operative control of a promoter specific for immature pre-B cells or mature germinal center (GC) B lymphocytes. In another embodiment, the placing of the recombinase coding sequence under the control of a promoter specific of B lymphocytes is achieved by inserting the recombinase coding sequence into the cγ1 locus or into the mb1 locus, or wherein the recombinase coding sequence is under the control of the Cgamma1 protein (Cγ1-cre) promoter or of the Cd79a protein (mb1-cre) promoter. In further embodiments, the genome of the engineered mouse comprising a bicistronic gene construct or a vector according to the invention integrated in its genome contains a single copy of the sequence encoding the recombinase.

In a particular embodiment, the genetically engineered mouse comprising a bicistronic gene construct or a vector according to the invention integrated in its genome is a mouse.

In another embodiment, the genetically engineered mouse comprising a bicistronic gene construct or a vector according to the invention integrated in its genome comprises the bicistronic gene construct is inserted into the Rosa26 locus.

Mouse Models Suffering from Multiple Myeloma

Within the context of the present invention, the genetically engineered mice according to the invention, as a result of the activation of the conditionally activatable genetic lesions, may develop human-like multiple myeloma (MM).

Thus, in a sixth aspect, the invention relates to a genetically engineered mouse according to the invention comprising within its genome the sequence encoding for a recombinase, as mentioned above, suffering from human-like multiple myeloma (MM).

By “multiple myeloma” (MM) it is meant any type of B-cell malignancy characterized by the accumulation of clonal terminally differentiated B-cells (plasma cells) in the bone marrow that secrete monoclonal immunoglobulins to serum. According to at least some embodiments, the multiple myeloma is selected from the group consisting of multiple myeloma cancers which produce light chains of kappa-type or light chains of lambda-type; and/or aggressive multiple myeloma, including primary plasma cell leukemia (PCL); and/or optionally including benign plasma cell disorders such as MGUS (monoclonal gammopathy of undetermined significance) which may proceed to multiple myeloma; and/or smoldering multiple myeloma (SMM), and/or indolent multiple myeloma, premalignant forms of multiple myeloma which may also proceed to multiple myeloma; and/or primary amyloidosis. With regard to premalignant or benign forms of the disease, optionally the compositions and methods thereof may be applied for prevention, in addition to or in place of treatment, for example optionally to halt the progression of the disease to a malignant form of multiple myeloma.

Within the context of the present invention, the genetically engineered mice of the invention will spontaneously develop MM. The evaluation of the presence of the disease in the mouse can be evaluated by the skilled in the art based on the general knowledge in the field.

In a particular embodiment, the appearance of the MM is detected by:
(i) detection of the expression of the reporter gene in plasma cells, and/or
(ii) detection of CD138 positive and CD19 negative plasma cells, and/or
(iii) detection of gamma fraction of the immunoglobulins IgG, IgA and/or IgM into serum, and/or
(iv) detection of at least one clinical feature characteristic of MM.

The term “detection” is used herein to refer to the identification of the presence of a feature or characteristic. When referred to the detection of a marker or expression level of a gene, is used to refer to a measurable quantity of a gene product or the reporter gene in a sample of a subject, wherein the gene product can be a transcriptional product or a translational product. As understood by the person skilled in the art, the gene expression level can be quantified by measuring the messenger RNA levels of said gene or of the protein encoded by said gene.

The level of a protein can be determined by any method known in the art suitable for the determination and quantification of a protein in a sample. By way of a non-limiting illustration, the level of a protein can be determined by means of a technique which comprises the use of antibodies with the capacity for binding specifically to the assayed protein (or to fragments thereof containing the antigenic determinants) and subsequent quantification of the resulting antigen-antibody complexes, or alternatively by means of a technique which does not comprise the use of antibodies such as, for example, by techniques based on mass spectroscopy. The antibodies can be monoclonal, polyclonal or fragment thereof, Fv, Fab, Fab′ and F(ab′)2, scFv, diabodies, triabodies, tetrabodies and humanized antibodies. Similarly, the antibodies may be labelled. Illustrative, but non-exclusive, examples of markers that can be herein used include radioactive isotopes, enzymes, fluorophores, chemoluminescent reagents, enzyme cofactors or substrates, enzyme inhibitors, particles, or dyes. There is a wide variety of known tests that can be used according to the present invention, such as combined application of non-labelled antibodies (primary antibodies) and labelled antibodies (secondary antibodies), Western blot or immunoblot, ELISA (enzyme-linked immunosorbent assay), RIA (radioimmunoassay), competitive EIA (enzyme immunoassay), DAS-ELISA (double antibody sandwich ELISA), two-dimensional gel electrophoresis, capillary electrophoresis, immunocytochemical and immunohistochemical techniques, immunoturbidimetry, immunofluorescence, techniques based on the use of biochips or protein microarrays including specific antibodies or assays based on the colloidal precipitation in formats such as reagent strips and assays based on antibody-linked quantum dots. Other forms of detecting and quantifying proteins include, for instance, affinity chromatography techniques or ligand-binding assays.

The expression CD138, also known as syndecan 1 or SYND1 refers to a transmembrane (type I) heparn sulfate proteoglycan which mediates cell binding, cell signaling and cytoskeletal organization. It functions as an integral membrane protein and participates in cell proliferation, cell migration and cell cell-matrix interactions via its receptor for extracellular matrix proteins.

The term CD19, also known and B-Lymphocyte Surface Antigen B4 or B-Lymphocyte Antigen CD19 makes reference to a surface protein of B cell lymphocytes. The protein has two N-terminal extracellular Ig-like domains separated by a non-lg-like domain, a hydrophobic transmembrane domain, and a large C-terminal cytoplasmic domain. This protein forms a complex with several membrane proteins including complement receptor type 2 (CD21) and tetraspanin (CD81) and this complex reduces the threshold for antigen-initiated B cell activation.

The expression plasma cells refers to plasma B cells or plasmocytes which develop from mature B lymphocytes (B cells), and are normally involved in secreting antibodies in order to fight foreign elements in the body (e.g., bacteria or virus infections).

The expression “clinical features characteristic of MM” include, without limitation bone pain with bone lytic lesions, an increased total serum protein concentration and/or the presence of a monoclonal protein in the urine or serum, systemic signs or symptoms suggestive of malignancy, such as unexplained anemia or hypercalcemia.

In a particular embodiment, appearance of the MM is additionally detected by the detection of at least one marker characteristic of MM selected from acid phosphatase, BCMA, SLAMF7 and TACI, and/or wherein the clinical feature characteristic of MM is a CRAB-like feature.

The term “acid phosphatase”, also known as “plasma cell acid phosphatase (PCAP)” is identifiable as the isoenzyme 3a, which is also detected in the cytoplasm of lymphocytes and platelets, where it shows a remarkable affinity for Naphtol AS-BI phosphate as substrate.

BCMA, also known as TNF Receptor Superfamily Member 17, is a receptor preferentially expressed in mature B lymphocytes, and may be important for B cell development and autoimmune response. This receptor has been shown to specifically bind to the tumor necrosis factor (ligand) superfamily, member 13b (TNFSF13B/TALL-1/BAFF), and to lead to NF-kappaB and MAPK8/JNK activation. This receptor also binds to various TRAF family members, and thus may transduce signals for cell survival and proliferation.

SLAMF7, also known as Membrane Protein FOAP-12, CD2-Like Receptor Activating Cytotoxic Cells or CD319, is a self-ligand receptor of the signaling lymphocytic activation molecule (SLAM) family. SLAM receptors triggered by homo- or heterotypic cell-cell interactions are modulating the activation and differentiation of a wide variety of immune cells and thus are involved in the regulation and interconnection of both innate and adaptive immune response.

The term TACI relates to a lymphocyte-specific member of the tumor necrosis factor (TNF) receptor superfamily. It interacts with calcium-modulator and cyclophilin ligand (CAML). The protein induces activation of the transcription factors NFAT, AP1, and NF-kappa-B and plays a crucial role in humoral immunity by interacting with a TNF ligand.

The term CRAB or CRAB-like feature refers to a series of symptoms associated with MM. C.R.A.B symptoms are signs of multiple myeloma. C.R.A.B is an acronym for Calcium elevation, Renal insufficiency, Anemia and Bone abnormalities.

Calcium elevation, or hypercalcemia, is when an individual has high levels of calcium in their blood. About 28% of people with multiple myeloma have hypercalcemia. Common symptoms include: nausea, vomiting, poor appetite, constipation, increased thirst, muscle weakness or twitching, fatigue, mental confusion and bone pain.

Renal insufficiency refers to poor kidney function, which may happen as a result of reduced blood flow to the kidneys. Some symptoms of poor kidney function include: fatigue, difficulty concentrating and sleeping, dry and itchy skin, frequent urination, blood in the urine, foamy urine, puffy eyes, swollen ankles and feet, poor appetite, muscle cramps, decreased urine output, dark urine.

Anemia is the term for a low red blood cell (RBC) count. It affects about 73% of people with multiple myeloma. These blood cells transport oxygen around the body. If a person's RBC count is too low, their tissues and organs do not receive enough oxygen. Symptoms of anemia include: feeling cold, dizziness or lightheadedness, fatigue, irritability, shortness of breath, frequent headaches, chest pain, pallor, which will be more apparent in people with light skin.

Bone abnormalities may include lesions or areas of damage. They can lead to symptoms such as: bone pain, particularly in the back and hips, bone weakness, osteoporosis, frequent bone fractures.

### Methods for the Induction of Multiple Myeloma in the Mice of the Invention

In a seventh aspect, the invention relates to a method for the induction of multiple myeloma (MM) in a mouse according to the invention, comprising within its genome the sequence encoding for a recombinase, said method comprising maintaining the mouse under conditions adequate for the development of multiple myeloma.

The method of the invention is performed in:
mice according to the invention comprising within its genome a sequence encoding for a recombinase which is specific for the recombinase target sites flanking the transcription terminator sequence within the conditionally activatable transgene or transgenes,
genetically engineered mice comprising the bicistronic gene construct or cassette or the vector of the invention within its genome and further comprising a sequence encoding for a recombinase which is specific for the recombinase target sites flanking the transcription terminator sequence within the conditionally activatable cistron.
Mice which have been administered a population of MM cells isolated from mice of the invention suffering MM and, optionally expanded and selected for the presence of the genetic lesions.

In a preferred embodiment, the conditions adequate for the development of multiple myeloma comprise the administration of an agent adequate for the induction of the proliferation of plasma cells.

Agents that induce proliferation of myeloma cells include, without limitation, anti-IgM molecule, soluble IL6 or IL10, and monoclonal antibodies against CD40 and CD40L.

In an embodiment, the administration of an agent adequate for the induction of the proliferation of plasma cells results in T cell-mediated immunization.

In a preferred embodiment, the T-cell mediated immunization is carried out using sheep red blood cells.

In another preferred embodiment, the T-cell mediated immunization comprises a priming immunization and at least one boost immunization.

Most vaccines are administered in two or more doses, the first one necessary for priming the immune system and generating cells able to fight the infection, such as plasma cells releasing antibodies, and the second one that boosts the primary response increasing the quality and the magnitude of the pathogen-specific immune response. This approach gives rise to the concept of prime-boost that is important not only in terms of improving the magnitude and duration of the response but also the quality.

Methods of immunization are known in the art. As an example, to induce the formation of GFP⁺ transgenic PCs in mice housed under specific pathogen-free conditions, mice are subjected to T cell-mediated immunization with sheep red blood cells (SRBCs), which are prepared in a 1×10¹⁰/ml solution of 100% stock solution (Fitzerald) diluted in Dulbecco's phosphate-buffered saline (DPBS). Mice are intraperitoneally (i.p.) administered 100 μl of the SRBC solution at eight weeks of age and are injected again every 21 days for 4 months. After immunization, a fraction of six-month-old mice from each cohort (n=4-6) are necropsied and analyzed to determine the presence and characteristics of B cells and PCs in spleen and bone marrow.

As mentioned above in relation to the genetically engineered mouse suffering from human-like multiple myeloma (MM), the appearance of the MM is detected by:
(i) detection of the expression of the reporter gene in plasma cells, and/or
(ii) detection of CD138 positive and CD19 negative plasma cells, and/or
(iii) detection of gamma fraction of the immunoglobulins IgG, IgA, and/or IgM into serum, and/or
(iv) detection of at least one clinical feature characteristic of MM.

In a particular embodiment, the method according to claim 6465 wherein the appearance of the MM is additionally detected by the detection of at least one marker characteristic of MM selected from acid phosphatase, BCMA, SLAMF7 and TACI, and/or wherein the clinical feature characteristic of MM is a CRAB-like feature.

In an eighth aspect, the invention relates to a genetically engineered mouse suffering from human-like MM which has been obtained by a method according to the seventh aspect of the invention.

In a ninth aspect, the invention relates to a MM cell population obtained from a mouse as defined in the eighth aspect of the invention.

Methods for the screening of candidate substance for the treatment of a MM using the mouse models according to the invention

In a tenth aspect, the invention relates to a method for the screening for a candidate substance for the treatment of a MM, which method comprises the steps of:
(i) providing a genetically engineered mouse according to the first or second aspect of the invention as far as they comprise within its genome a sequence encoding for a recombinase which is specific for the recombinase target sites flanking the transcription terminator sequence within the conditionally activatable transgene or transgenes, or in a mouse according to the fifth aspect of the invention as far as it additionally comprises within its genome a sequence encoding for a recombinase which is specific for the recombinase target sites flanking the transcription terminator sequence within the conditionally activatable cistron, and inducing in said mouse the proliferation of plasma cells until the presence of MM is detected, providing a mouse suffering from MM according to the sixth aspect of the invention or implanting into a mouse the MM cell population as defined in the ninth aspect of the invention under conditions adequate for the engraftment of said MM cell population;
(ii) administering said candidate substance to said mouse;
(iii) determining the effect of the candidate substance on the MM;
wherein an increased effect of the candidate compound on the MM with respect to the effect observed in a mouse treated with a control substance indicates that the candidate compound is suitable for the treatment of MM.
Accordingly, the method according to the tenth aspect of the invention can be carried out in:
   mice according to the invention comprising within its genome a sequence encoding for a recombinase which is specific for the recombinase target sites flanking the transcription terminator sequence within the conditionally activatable transgene or transgenes,
   genetically engineered mice comprising the bicistronic gene construct or cassette or the vector of the invention within its genome and further comprising a sequence encoding for a recombinase which is specific for the recombinase target sites flanking the transcription terminator sequence within the conditionally activatable cistron.

In these cases, mice need to be treated under conditions adequate for the proliferation of plasma cells until the presence of MM is detected

The method can also be performed in an suffering from MM according to the invention as well as in mice which have been administered a population of MM cells isolated from the mice of the invention suffering MM and, optionally expanded and selected for the presence of the genetic lesions. In this case, the mice already suffer from MM and thus, the step of inducing the appearance of MM is not strictly required.

In a particular embodiment, the effect of the candidate compound on the MM is measured by detecting changes in one or more of:
(i) the number of plasma cells with the expression of the reporter gene in the bone marrow or the peripheral blood, and/or
(ii) the levels of at least one marker characteristic of MM, and/or
(iii) the level of at least one clinical feature characteristic of MM

In another embodiment, the marker characteristic of MM is acid phosphatase, BCMA, SLAMF7, TACI, secreted Igs or content of the Ig γ globulin protein fraction (M-spikes) in serum and/or wherein the clinical feature characteristic of MM is a CRAB-like feature.

M-spikes refer to M are antibodies that are present in the blood. People with multiple myeloma or another plasma cell disorder can have high levels of clonal M proteins in the blood. M proteins are known by several different name monoclonal immunoglobin, paraproteins, monoclonal proteins or M spike. M proteins can be heavy chains or light chains. M proteins can be made up of one type of heavy chain and one type of light chain. Heavy chains comprising: immunoglobulin A (IgA), immunoglobulin M (IgM), immunoglobulin E (IgD), immunoglobulin G (IgG) and immunoglobulin D (IgE); and light chains comprising kappa and lambda chains.

M proteins always produce the same types of cells. So, if a person has IgA heavy chains and kappa light chains, all of their proteins will be IgA kappa.

In a particular embodiment, the effect of the candidate compound on the MM is measured by an MM end-point marker.

In a preferred embodiment, the said end-point is the median overall survival (OS) of said genetically engineered mouse.

The median overall survival (OS) refers to the length of time from either the date of diagnosis or the start of treatment for a disease, such as cancer, that half of the patients in a group of patients diagnosed with the disease are still alive.

In an eleventh aspect, the invention relates to the use of a genetically engineered mouse according to the first or second aspect of the invention as far as they comprise within its genome a sequence encoding for a recombinase which is specific for the recombinase target sites flanking the transcription terminator sequence within the conditionally activatable transgene or transgenes and/or specific for the recombinase target sites present in the Trp53 gene region which is required for the expression of a functional Trp53 protein, or in a mouse according to the fifth aspect of the invention as far as they additionally comprises within its genome a sequence encoding for a recombinase which is specific for the recombinase target sites flanking the transcription terminator sequence within the conditionally activatable cistron, a mouse suffering from MM according to the sixth aspect of the invention or implanting into a mouse the MM cell population as defined in the ninth aspect of the invention under conditions adequate for the engraftment of said MM cell population, for the screening a candidate substance for the treatment of a MM.

### Genetically Engineered Mouse Comprising a Conditionally Activatable Transgene Encoding the Human MMSET-II Protein

In a twelfth aspect, the invention relates to genetically engineered mouse comprising a conditionally activatable transgene encoding the human MMSET-II protein, wherein the conditionally activatable transgene comprise a transcription terminator sequence flanked by recombinase target.

In another embodiment, the transcription terminator sequence is a polyA sequence.

In another embodiment, the transgene is under operative control of a CAG promoter.

In a particular embodiment, the conditionally activatable transgene encoding the MMSET-II protein is inserted in the Rosa26 locus.

In another embodiment, the genetically engineered mouse according the twelfth aspect of the invention contains a single copy of the conditionally activatable transgene encoding the MMSET-II protein.

In a preferred embodiment, the conditionally activatable transgene further comprises a reporter gene that is co-expressed upon activation of the transgene.

In another embodiment, reporter gene that is co-expressed upon activation of the transgene is a gene encoding a fluorescent protein or the human CD2 gene.

In another embodiment, the conditionally activatable transgene and the reporter gene are found within the same gene construct under the control of the same promoter and wherein an IRES is inserted between the conditionally activatable transgene and the reporter gene.

In another embodiment, the genetically engineered mouse according to twelfth aspect of the invention additionally comprises within its genome a sequence encoding for a recombinase which is specific for the recombinase target sites flanking the transcription terminator sequence within the conditionally activatable transgene or transgenes.

In another embodiment, the recombinase is the Cre recombinase and the recombinase target sites are loxP sites.

The invention will be described by way of the following examples which are to be considered as merely illustrative and not limitative of the scope of the invention.

### Examples

### Materials and Methods

### Mouse Strains

Eight transgenic mouse strains carrying common MM genetic changes were used. Five were obtained from The Jackson Laboratory (Bar Harbor, ME, USA): B6(Cg)-Gt(ROSA)26Sor^{tm4(lkbkb)Rskyl}J mice with constitutively active NF-κB signaling by IKK2 expression and a green fluorescent protein (GFP) reporter; 129S/Sv-Kras^{tm4Tyj}/J mice with the Kras^{G12D} mutation; B6.Cg-Tg(BCL2)22Wehi/J mice with BCL2 expression; C57BL/6N-Gt(ROSA)26Sor^{tm13(CAG-MYC,-CD2*)Rsky}/J mice with c-MYC expression and a truncated human CD2 reporter; and B6.129P2-Trp53^{tm1Brn}/J mice with p53 deletion. The two previously reported mouse strains Cg-Tg (Eu-CCND1) and B6.Cg-Tg (Eu-C-MAF), which represent t(11; 14) and t(14; 16), respectively, were also used. Finally, Rosa26-hMMSET-IIStop-Floxed mice were generated as a model of t(4; 14). To establish this model, a construct encoding human MMSET-II cDNA preceded by a loxP-flanked STOP cassette was integrated into the mouse Rosa26 locus (using Addgene plasmid 15912). Consequently, transgene transcription is controlled by a CAG promoter, and its expression can be detected by GFP expression, which is placed under control of an internal ribosomal entry site (IRES) downstream of the cDNAs. The linearized targeting vector was transfected into mouse ES cells, and targeted clones were isolated using positive (NeoR) selection. Correct integration was verified by Southern blot of EcoRI-digested genomic DNA from mouse ES cells and founder mouse tails using a Rosa26-specific probe (external Rosa probe A) and by PCR. Transgenic activation was obtained by crossing mice with two cre-recombinase mouse lines: mb1-cre mice, kindly provided by Prof. Michael Reth (University of Freiburg), and cγ1-cre mice (B6. 129P2(Cg)-Ighg1^{tm1(cre)Cgn}/J) obtained from the Jackson laboratory. As controls, mb1-cre or cγ1-cre mice crossed to B6.129X1-Gt(ROSA)26Sor^{tm1(EYFP)Cos}/J mice (The Jackson Laboratory), which carry a yellow fluorescent protein (YFP) reporter, were generated. The Vk*MYC mice, which die of human-like MM at late age, were also included as a positive disease control. Strains were intercrossed by conventional breeding to obtain the corresponding compound mice with heterozygous or homozygous alleles, which were maintained in a hybrid C57BL6/129Sv genetic background. Mice were kept under specific pathogen free conditions in the animal facilities of the Center for Applied Medical Research CIMA at the University of Navarra. Animal experimentation was approved by the Ethical Committee of Animal Experimentation of the University of Navarra and by the Health Department of the Navarra Government.

### Genetic Screens and Immunization Protocol

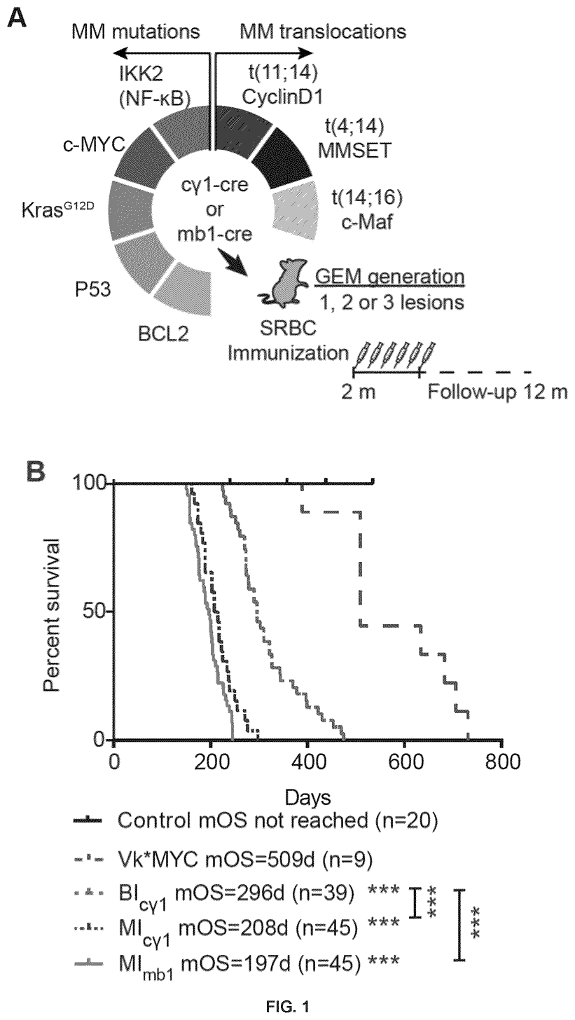

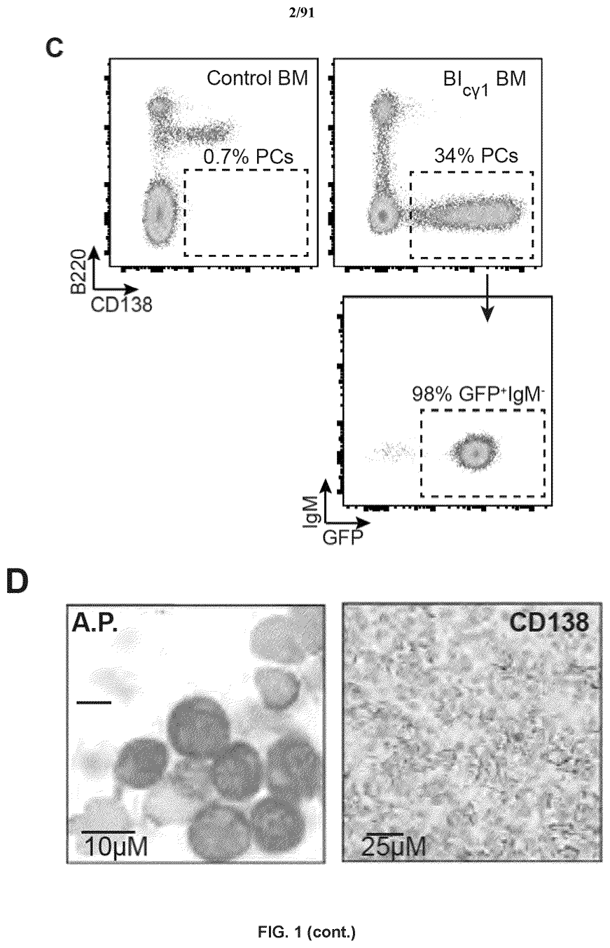

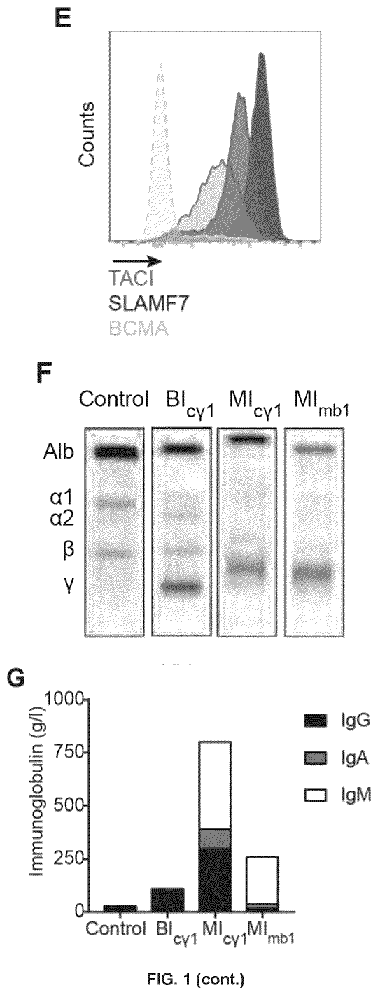

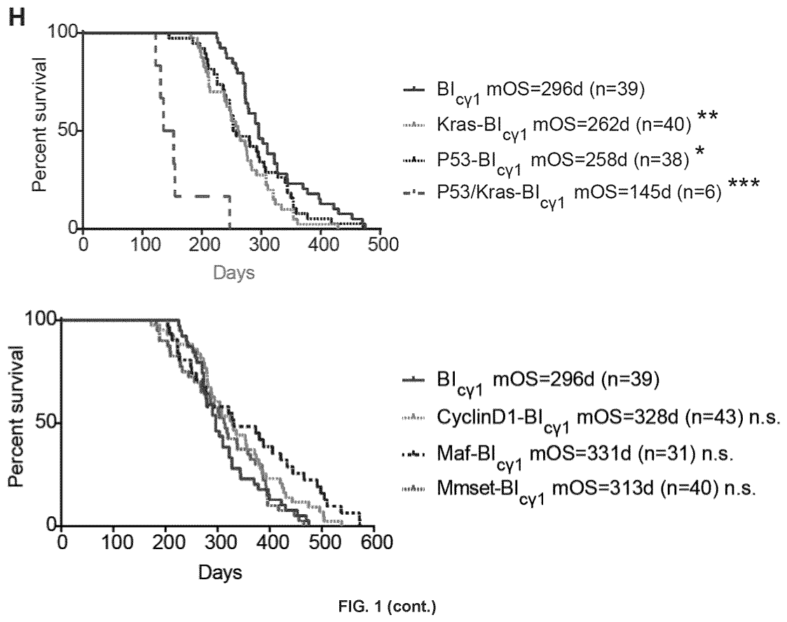

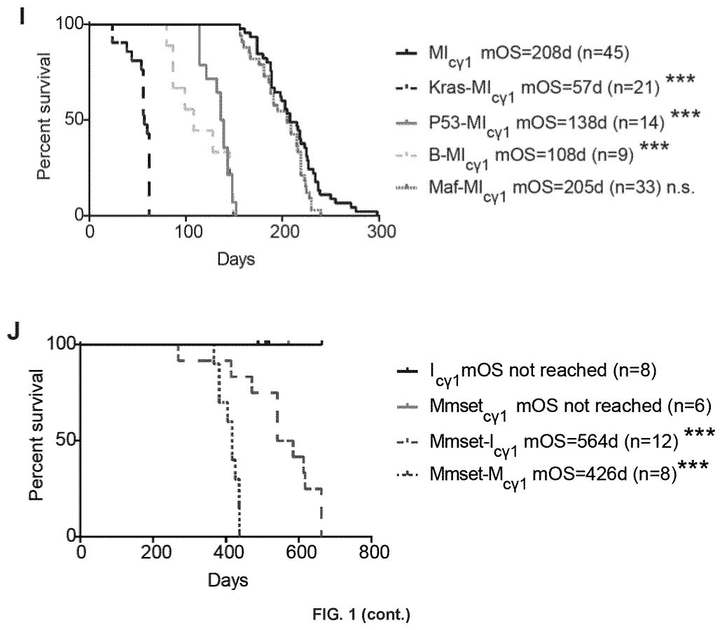

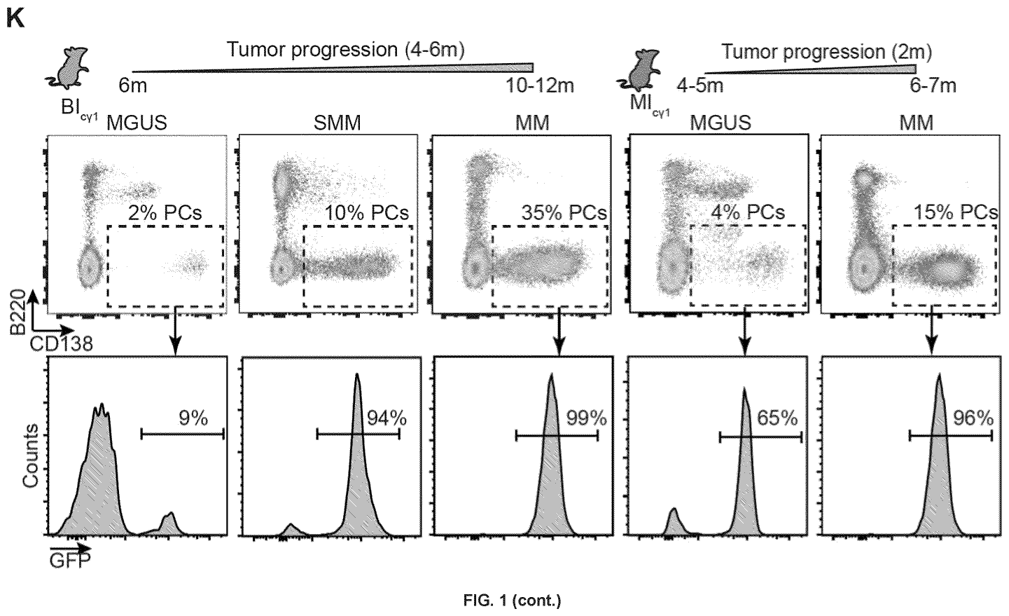

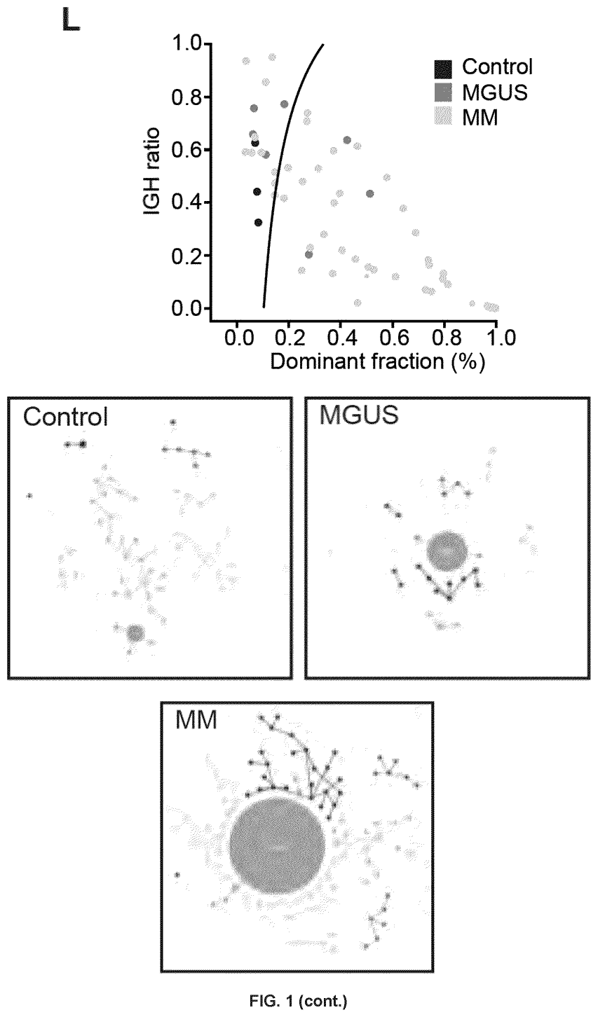

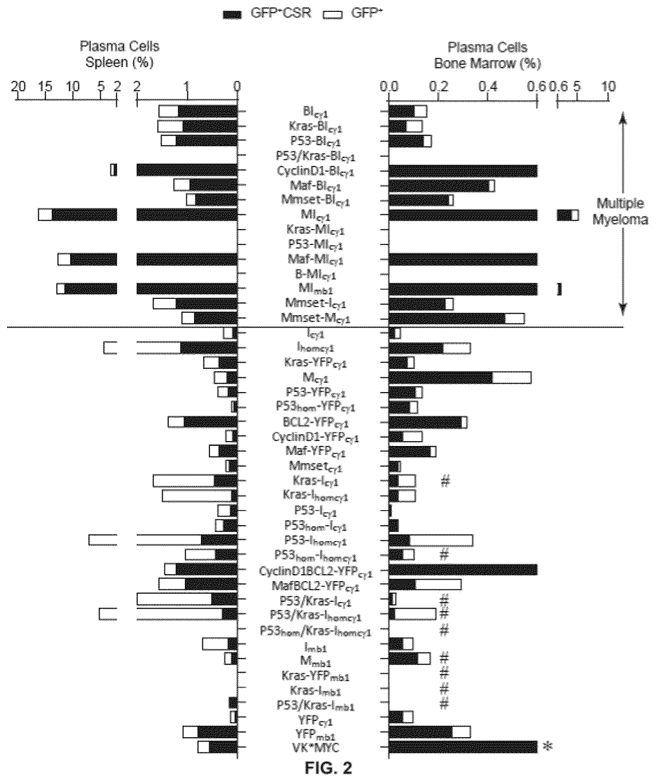

To model MM genetic heterogeneity, the eight strains of transgenic mice carrying MM genetic drivers were bred to engineer strains with single, double, or triple genetic alterations. Genetic abnormalities were triggered in immature pre-B or mature GC B lymphocytes using mb1-cre or cγ1-cre mice, respectively. To induce the formation of GFP⁺ transgenic PCs in mice housed under specific pathogen free conditions, animals were subjected to T cell-mediated immunization with sheep red blood cells (SRBCs), which were prepared in a 1×10¹⁰/ml solution of 100% stock solution (Fitzerald) diluted in Dulbecco's phosphate-buffered saline (DPBS). Mice were intraperitoneally (i.p.) administered 100 μl of the SRBC solution at eight weeks of age and were injected again every 21 days for 4 months. After immunization, a fraction of six-month-old mice from each cohort (n=4-6) were necropsied and analyzed to determine the presence and characteristics of B cells and PCs in spleen and bone marrow (FIG. 2). The remaining mice from each cohort were monitored for tumor development up to 12 months of age. Similarly immunized YFPmb1 or YFPcγ1mice and Vk*MYC mice were characterized as controls. Survival rates of these diverse mouse strains were 48 estimated using Kaplan-Meier overall survival curves.

### Flow Cytometry Analyses and Cell Sorting

Cell suspensions from spleen (obtained by mechanical disruption) and bone marrow (flushed from femurs with DPBS) were filtered through a 70-μm cell strainer (Falcon) and treated with ACK lysis buffer to remove red blood cells. Then, cells were washed in DPBS and filtered a second time before they were labeled with antibodies for flow cytometric analysis. Mouse antibody panels, which are listed in the Key Resources Table, were used to detect tumor and immune cell subpopulations. Data acquisition was performed in a FACS Cantoll flow cytometer (BD Biosciences) and analyzed using FlowJo™ 56 V10.7.1 software. For cell sorting, stained cells were separated using a FACS Aria sorter instrument (BD Biosciences). Characterization of the BM microenvironment was performed by flow cytometry in 17 control, 31 MGUS and 59 MM mice representing the different genetic subgroups. Immune infiltration of the BM was evaluated according to the percentages of T cells (CD4 plus CD8) and NK cells present in the non-tumor fraction. Mice presenting an immune infiltration similar to that of control age-matched mice were considered immune-cold cases (cut off value, 1.8 times the mean value in the control group), while tumors with higher percentages of T and NK cells were classified as having an immune-inflamed microenvironment.

### Serum Protein Electrophoresis and Enzyme-Linked Immunosorbent Assay (ELISA)

Sera were extracted from blood obtained by puncture of the submandibular vein and collected in a Microvette Z gel tube (Sarstedt). A 5-μl fraction was applied to an agarose gel (HYDRAGEL 30 Protein), which was analyzed in a semi-automated Hydrasys 2 device; this device quantified the serum protein components that were separated into five fractions by size and electrical charge. The gamma-globulin (γ) fraction in diseased mice was measured and compared with that in control aged-matched mice. In selected samples, an isotyping multiplex assay was used to simultaneously quantify Ig isotypes in serum using the MILLIPLEX® Mouse Immunoglobulin Isotyping kit on the Luminex® xMAP® platform.

### Laboratory Analyses

Hemogram tests were performed with 50 μl of blood collected in a Microvette EDTA tube (Sarstedt) using an Element HT5 (CMV Diagnóstico Laboratorio) instrument. Creatinine, calcium, and urea levels were detected by standard laboratory methods in a Cobas 8000 analyzer (Roche Diagnostics) at the Biochemistry Laboratory of the Clinic University of Navarra.

### Examination of Bone Lesions

Long mouse bones were examined using three-dimensional tomographic images acquired by X-ray micro-CT (Quantum-GX, Perkin Elmer). The 3D tomographic images contained 512 slices with an isotropic 50-μm voxel size and a resolution of 512×512 pixels per slice. To perform the bone histomorphometry analysis, a region of interest (ROI) containing the bone diaphysis and epiphysis (15×15×15 mm) was reconstructed from the original scan at a resolution of 30 μm/voxel using Quantum 3.0 software. Bone mineral density analysis in each ROI was performed using a plugin developed for Fiji/ImageJ. Studies were performed at the Imaging Platform at the Center for Applied Medical Research of the University of Navarra.

### IgVH Gene Clonality

Two different strategies were used. First, IgHV gene rearrangements were amplified by PCR in genomic DNA isolated from GFP⁺-sorted MM cells and peripheral blood CD19⁺ B lymphocytes from healthy mice using specific VHA, VHE, and VHB forward primers and a reverse primer for JH4, which are listed in the Key Resources Table. Individual fragments were purified from gel or directly from the PCR reaction mixture using NucleoSpin Gel and PCR Clean-up (Macherey-Nagel), sequenced, and blasted against the ImMunoGeneTics information System® using the tool found at http://www.imgt.org/IMGT_vquest to determine VDJ usage. The second strategy consisted of the analysis of IgH gene clonality from the RNA-seq analysis in YFP-sorted BM PCs from control YFPcγ1 mice and in GFP+BM tumor cells from mice in the MGUS and MM states, through B-cell receptor (BCR) reconstruction using Mixcr tool. Briefly, raw fastq data were analyzed by mixcr v3.0.12 to reconstruct the BCR clonality based on the CDR3 clonotypes frequencies separately in IGH, IGK, and IGL chains according to previously reported methods.

### Immunohistochemistry (IHC)

Spleen, bone, and kidney tissues were fixed in 4% (wt/vol) paraformaldehyde (Panreac) for 48-72 h and washed in 70% ethanol before paraffin embedding. Tissue sections were stained with hematoxylin & eosin and with specific monoclonal antibodies (listed in the Key Resources Table). An automated immunostaining platform (Discovery XT-ULTRA, Ventana-Roche) was used. Briefly, sections stained with rat anti-CD138 (clone 281-2; 1/20,000) were incubated with rabbit anti-rat secondary antibody (BA4001, 1/100). Then, the sections were incubated with goat anti-rabbit-labeled polymer using the EnVisionTM+ System (Dako), and peroxidase activity was revealed using DAB+ (Dako). For stains with monoclonal anti-c-MYC (Y69, 1/100) or anti-GFP (D5.1, 1/100), slides were incubated with the visualization systems (OmniMap anti-Rabbit) conjugated to horseradish peroxidase. IHC reactions were developed using 30-diaminobenzidine tetrahydrochloride (DAB) (ChromoMap DAB, Ventana, Roche) and purple chromogen (Discovery Purple Kit, Ventana, Roche). Finally, nuclei were counterstained in Hematoxylin II. In selected BM samples, Giemsa or alkaline phosphatase staining was performed according to standard procedures.

### Quantitative RT-PCR

A total of 1 μg of total RNA from GFP⁺-sorted cells was isolated with a NucleoSpin RNA kit (Macherey-Nagel) and reverse transcribed into cDNA using MMLV enzyme technology (Invitrogen). PCR was performed on an ABI Viia7 instrument using SYBR green fluorophore and primers designed to amplify specific mouse or human genes. Specific primers are listed in the Key Resources Table.

### MM Patient Samples

Clinical BM aspirate samples from patients with newly diagnosed MGUS (n=108), SMM (n=167), or MM (n=652) were analyzed by multi-parametric flow cytometry. In addition, 9 MGUS and 41 MM samples from newly diagnosed patients were characterized by RNA seq. BM aspirates from 24 adult donors, ranging from younger to older ages (51 to 84 years; median age, 72.5 years), were included as controls. All samples were obtained from the University of Navarra Biobank. A series of 170 samples from patients with newly diagnosed MM enrolled in the PETHEMA/GEM-CLARIDEX clinical trial (NCT02575144) were characterized by multi-parametric flow cytometry. A series of patients with 69 newly diagnosed MM was included. This study was performed in accordance with the regulations of the Institutional Review Board of the University of Navarra and was conducted according to the principles of the Declaration of Helsinki. Informed consent was obtained from all patients.

### Flow Cytometry Analysis and Cell Sorting in Patient Samples

Characterization of patient samples was performed using the EuroFlow lyse-wash-and-stain using a standard sample preparation protocol adjusted to 10⁶ BM-derived nucleated cells, together with the 8-color combination of the monoclonal antibodies (mAb) CD138-BV421, CD27-BV510, CD38-FITC, CD56-PE, CD45-PerCPCy5.5, CD19-PECy7, CD117-APC, and CD81-APCH7 (BD Biosciences). Data acquisition was performed in a FACS Cantoll flow cytometer (BD Biosciences). Samples were analyzed using the Infinicyt software (Cytognos SL) and the semi-automated pipeline “FlowCT”, based on the analysis of multiple files by automated cell clustering. Cell sorting was performed in a FACS Aria sorter instrument. Classification of BM samples according to immune cell infiltration was calculated similar to that in the mouse samples. The maximum percentages of T and NK cells present in the BM from healthy control individuals (cut off 20%) were used to divide patients with MM into immune-cold and immune-inflamed cases.

### Human MM Cell Lines

Ten cell lines derived from MM patients (RPMI8226, KMS12, KMS26, KMS11, MM1S, U266, K620, JJN3, H929, and MOLP2) were included in this study. All have been validated according to the AmpFLSTR® Identifiler®, were tested for Mycoplasma sp.

Generation of MM-Derived Cell Lines from Primary Samples

Cell suspensions from BM and/or spleen samples from mice exhibiting MM development were injected through the tail vein of Rag2^{-/-}IL2γc^{-/-} immunodeficient mice (The Jackson Laboratory). Mice were monitored twice weekly for signs of disease and were then sacrificed. Upon serial transplantations, cases that predominantly exhibited GFP⁺CD138⁺B220⁻IgM⁻ PCs cells were selected, and the cells were expanded in vitro. The samples that were able to grow for weeks ex vivo were tested for the presence of the original transgenic lesions and then characterized. Eight MM-derived cells lines established from mice carrying different lesions. In vitro therapy assays. For viability assays, murine or human MM cells were seeded in 96-well black culture plates and treated with different drugs alone or in combination for 48 hours. Cell viability was quantified using a Deep Blue Cell Viability™ Kit (Biolegend) and analyzed with a Skanit Varioskan Flash 2.4.3 (Thermo Scientific) fluorometer. Treatments were administered to cells at a density of 0.3×10⁶ cells/ml, and all tests were performed in triplicate. After treatment, cells were subjected to qRT-PCR or Western blot analyses, as indicated, according to previously reported methods.

### Statistical Analysis

Statistical analyses were performed using GraphPad Prism 9.0. Two-tailed Student t tests or Mann-Whitney U tests were used to evaluate the statistical significance of differences in gene expression values and/or other quantifications. Mouse survival was estimated using Kaplan-Meier curves and compared using the log-rank test. Statistical values are indicated throughout the manuscript as follows: n.s. not significant, *p<0.05, **p<0.01, and ***p<0.001.

### RNA Sequencing (RNA-seg)

RNA-seq was performed in isolated BM GFP⁺CD138⁺B220⁻ PCs from mice at the MGUS (n=24) and MM (n=38) stages and in BM CD38⁺CD138⁺ PCs from patients with newly diagnosed MGUS (n=9) and MM (n=41). BM GFP⁺CD138⁺B220⁻IgM⁻ PCs (n=6) and spleen B220⁺CD38⁻FAS⁺ germinal center B cells (n=3) were isolated from T cell-immunized 6-month-old YFPcγ1 mice, and used as controls. In addition, human CD38⁺CD138⁺ PCs were isolated from BM aspirates from adult healthy donors (n=7). RNA-seq was performed on 20,000 cells per sample using a reported MARS-seq protocol adapted for bulk RNA-seq with minor modifications. Libraries were sequenced in an Illumina NextSeq 500 at a sequence depth of 10 million reads per sample. A second RNA-seq study was conducted in GFP⁺CD138⁺B220⁻ PCs isolated from 20 BM samples obtained from mice at the MGUS (n=1) and MM (n=19) stages. RNA was extracted from fresh-frozen samples maintained in TRIzol (Invitrogen), and libraries (PE 50 or 100 bp) were prepared using the TruSeq RNA sample kit and validated using an Agilent Technologies 2100 Bioanalyzer. Library preparation, sequencing, and post-processing of the raw data were performed on an Illumina HiSeq2500.

### Spectral Karyotyping and Fluorescence In Situ Hybridization (FISH)

Murine MM cells were cultured, harvested, and fixed according to standard cytogenetic protocols. Metaphase spreads from fixed cells were hybridized with the HiSKY probe (FPRPR0030). Slides were prepared for imaging using a CAD antibody kit (FPRPR0033, Applied Spectral Imaging) and counterstained with DAPI. Twenty metaphase spreads were then captured and analyzed using HiSKY software (Applied Spectral Imaging). Interphase FISH was performed according to standard protocols to determine chromosome 1q genomic amplification.

### Whole-Exome Sequencing (WES)

WES was performed in 71 BM samples isolated from GFP⁺CD138⁺B220⁻ PCs (purity, >99%), including 62 samples from the MM stage, 3 samples of pooled PCs from nine mice at the MGUS stage, and 6 samples from MM-derived cell lines. Six BM samples with YFP⁺CD138⁺B220⁻ PCs (purity, >99%) isolated from YFPcγ1 mice were also included. Genomic DNA was purified using a NucleoSpin® Tissue kit (Macherey-Nagel). DNA quality and concentration were evaluated using an Agilent 4200 Tape Station (Agilent) and a Qubit System (Invitrogen, Carlsbad, CA), respectively. Exome capture libraries were prepared according to the SureSelectXT mouse all exon target enrichment system (Agilent Technologies) and were sequenced using a 151 base pair paired-end read protocol by Macrogen on an Illumina Novaseq6000. Sequencing resulted in a mean read-depth of 112× (range 33-216×). The resulting FASTQ file analysis was performed with the Genome One platform (Dreamgenics, S. L.). Raw FASTQ files were evaluated using the FastQC and Trimmomatic quality controls. Each FASTQ was aligned with the GRCm38/mm10 version of the mouse genome reference with BWA-mem. Ordered BAM file generation was performed with SAMtools, and optic and PCR duplicate deletion was performed with Sambamba. Single nucleotide variants (SNVs) and indels were identified with the combination of VarScan 2, and Dreamgenics S.L developed an algorithm for variant calling. Variants were annotated with Ensembl functional information, mice population allelic frequencies from dbSNP, and an adaptation of the MGP database that did not include the wt mouse strain. Furthermore, a new database was generated with the variants identified in control mice. For potentially somatic preliminary variant selection in each sample, the following filters were applied: a) Variants with total coverage of the affected position >20X, reads/variant ≥6, and allelic frequency >0.1. b) Absence of variants in dbSNP, MGP, and the control database. c) Functional prediction of effects on protein. d) Absence of fault summary annotations. e) Frequency <0.05 of the variant in the sample. f) Number of reads with the variant <5. Potential CNV identification analysis was performed with a MoCaSeq adaptation of CopywriteR.

### Whole-Genome Sequencing (WGS)

WGS was performed in the two murine cell lines MM5080 and MM9275 and the corresponding matched germline DNAs. Briefly, genomic DNA was purified using a NucleoSpin® Tissue kit (Macherey-Nagel). DNA quality and concentration were evaluated with a Qubit System (Invitrogen). Next-generation sequencing (NGS) capture libraries were prepared according to the Truseq Nano DNA Library (Illumina) and were sequenced using a 150 base pair paired-end read protocol by Macrogen on an Illumina Novaseq6000. The resulting FASTQ file analysis was performed by the Genome One platform (Dreamgenics, S. L.) using the HMMcopy adaptation of CopywriteR.

### Bulk RNA-Seg and Bio-Informatic Deconvolution

These studies were performed following reported methods. Total BM samples from genetically diverse mice at MGUS (n=6) and MM (n=28) stages were included, along with six BM samples from healthy YFPcγ1 mice: Total BM samples from genetically diverse mice at MGUS (n=6) and MM (n=28) stages were included, along with six BM samples from healthy YFPcγ1 mice. For patient sample analyses, public RNA-seq and microarray datasets corresponding to total or CD138-depleted BM samples from newly diagnosed MM patients (n=426) in two clinical series were included: GSE13632421 and GSE10417122222.

Single-Cell RNA Plus TCR Sequencing (scRNA/TCRseq)

T cells were isolated by FACS based on expression levels of CD19, CD56, CD30e and CD3 for human cells and B220, CD3 and NK1.1 for mouse cells. scRNA/TCRseq was performed using 10× Genomics Single Cell 5′ Solution, version 2, according to the manufacturer's instructions (10× Genomics). Libraries were sequenced on NextSeq500 (Illumina) and analyzed using the Cell Ranger version 3.0.0 software (10× Genomics). Quality control metrics were used to select cells with mitochondrial genes representing <10% of total genes and with at least 200 genes. The final number of T cells characterized was as follows: 12, 197 T cells from BIcγ1 mice with MM (n=3); 11,124 T cells from BIcγ1 mice with MGUS (n=3); 5,331 T cells from the BM of 6 month-old YFPcγ1 mice (n=2); 32,988 T cells from newly diagnosed MM patients (n=7); 15,870 T cells from patients with MGUS (n=4); and 29,011 T cells from the BM of healthy adults (n=6). Samples were analyzed using Seurat (https://satijalab.org/seurat/). Clonotypic TCRs were defined based on their presence in 10 or more cells. To integrate different SCRNA/TCRseq samples we used a normalization and variance stabilization of molecular count data based on regularized negative binomial regression with a sctransfrom function. Results were shown by uniform manifold approximation and projection (UMAP) plots of single-cell transcriptomic and TCR genomic profiles.

### Pre-Clinical In Vivo Therapy Trials

These were performed in MIcγ1 and BIcγ1 mice. Before therapy initiation, tumor burdens were estimated by measuring the Ig γ fraction (M-spikes) in serum by electrophoresis. Animals of both sexes with similar tumor burdens were separated into experimental groups. Depletion studies or immunotherapy pre-clinical trials were initiated when MIcγ1 and BIcγ1 mice were 4 and 6 months of age, respectively. Monoclonal antibodies were administered by i.p. injection once weekly for 8 weeks. Mice received 200 μg of anti-PD1, anti-PD-L1, anti-TIGIT, or rat IgG control antibody. For depletion studies, 100 μg of anti-CD4, anti-CD8, or rat IgG control antibody was administered on days +1, +4, and +8 and then weekly for 8 weeks. Therapy responses were determined by comparing serum M-spikes at day 0 with those at 4 and 8 weeks after treatment initiation, and by median overall survival (OS). All therapeutic regimens were well tolerated, with no significant body weight loss or overt signs of toxicity other than those attributable to the tumor itself. Animals were monitored twice weekly to detect any signs of discomfort and/or disease, which included hunching, ruffled fur, belabored breathing, low body temperature, low mobility, and/or >20% weight loss from the time of study initiation. Survival was estimated by Kaplan-Meier curves and was compared using the log-rank test.

### MM-Derived Syngeneic Transplants and In Vivo Therapy

Establishment of syngeneic transplants was performed by injecting 5×10⁶ cells from established MM cell lines in DPBS into the tail veins of 8- to 10-week-old C57BL/6 mice. The MM8273 syngeneic model was established by subcutaneous injection of 10×10⁶ cells in DPBS in the flank of 8- to 10-week-old C57BL/6 mice. Upon injection of MM cells, animals of both sexes were randomly divided into experimental groups. Anti-CD4, anti-CD8, or rat IgG control antibodies (100 μg each) were administered at days +1, +4, +8, and +16 post-injection. To genetically deplete Treg cells, B6.129 FoxP3 DTR mice (The Jackson Laboratory) were injected with 250 ng of diphtheria toxin (DT) weekly for three weeks starting on day +1 post-injection. For immunotherapy studies, 200 μg of anti-PD1, anti-TIGIT, or anti-PDL1 monoclonal antibodies was i.p. injected twice weekly for 3 weeks starting on day +1 post-injection. Anti-CD25 antibody (clone 7D4 (CD25 NIB), moIgG2a isotype) was administered by i.p. injection starting on day +1 post-injection (75 μg/mouse) and continued weekly for three consecutive weeks. Therapy responses were estimated by Kaplan-Meier survival curves, which were compared using the log-rank test. In the subcutaneous MM8273 syngeneic models, therapy was started when tumors reached 400 mm3. Tumor growth was monitored every two days by measuring tumor size in two orthogonal dimensions using a caliper. Tumor volume was calculated using the formula V=(L2×W)/2.

### Results

### Modeling Genetic Heterogeneity of Human MM in Mice

To establish pre-clinical models of genetically heterogeneous MM, transgenic mice carrying eight MM genetic drivers that recapitulate the most common changes observed in human MM were bred to engineer strains with single, double, and triple genetic alterations. These included NF-κB signaling activation by IKK2 expression, a KrasG12D mutation, anti-apoptotic BCL2 expression, c-MYC expression, P53 deletion, and constitutive expression of Cyclin-D1, c-MAF, and MMSET mimicking Ig translocations t(11;14), t(16;14), and t(4;14), respectively. These changes were triggered in immature pre-B or mature germinal center (GC) B lymphocytes, which are the two developmental stages proposed to be the origin of the disease, using mb1-cre or cγ1-cre recombinase mice, respectively. Young mice were immunized with sheep red blood cells to induce the formation of PCs labeled with a green fluorescent protein (GFP) reporter; mice were then monitored for MM development up to 12 months of age (FIG. 1a and FIG. 2). Vk*MYC mice were included as a reference model of MM development at a late age, driven by single MYC expression in GC B lymphocytes.

The following Table shows the outcome of the genetic screen performed by activating different groups of two, three and four different cancer genetic lesions resulting from the combination of an initial set of eight single lesions in combination with two mouse B cell stages.

| | | No of | | Median OS | No of | |
|---|---|---|---|---|---|---|
| Mouse model | Genetic lesions | lesions | Cre-rec | (days) | mice | Disease |
| BI_{cγ1} | BCL2Eμ Ikk2stopF/WT | 2 | cγ1-cre | 296 | 39 | Multiple myeloma |
| MI_{cγ1} | MYCstopF/WT Ikk2stopF/stopF | 2 | cγ1-cre | 208 | 45 | Multiple myeloma |
| MMSET-Icγ1 | MMSETstopF/WT Ikk2stopF/WT | 2 | cγ1-cre | 564 | 12 | Multiple myeloma |
| MMSET-Mcγ1 | MMSETstopF/WT MYCstopF/WT | 2 | cγ1-cre | 426 | 12 | Multiple myeloma |
| MI_{mb1} | MYCstopF/WT Ikk2stopF/WT | 2 | mb1-cre | 197 | 45 | Multiple myeloma IgM |
| Kras-I_{cγ1} | KrasG12D/WT Ikk2stopF/WT | 2 | cγ1-cre | 365 | 4 | Not observed |
| Kras-I_{homcγ1} | KrasG12D/WT Ikk2stopF/stopF | 2 | cγ1-cre | 336 | 20 | B-cell lymphoma/T-ALL |
| Trp53-Icγ1 | Trp53F/WT Ikk2stopF/WT | 2 | cγ1-cre | >365 | 10 | Not observed |
| Trp53hom-Icγ1 | Trp53F/F Ikk2stopF/WT | 2 | cγ1-cre | not | — | Not observed |
| | | | | evaluated | | |
| Trp53-Ihomcγ1 | Trp53F/WT Ikk2stopF/stopF | 2 | cγ1-cre | 291 | 9 | not observed |
| Trp53hom-Ihomcγ1 | Trp53F/F Ikk2stopF/stopF | 2 | cγ1-cre | 230 | 25 | B-cell lymphoma |
| CyclinD1B-YFP_{cγ1} | CyclinD1^{Eμ} BCL2^{Eμ} YFP^{stopF/WT} | 2 | cγ1-cre | >365 | 6 | Not observed |
| MafB-YFP_{cγ1} | MAF^{Eμ} BCL2^{Eμ} YFP^{stopF/WT} | 2 | cγ1-cre | not | — | Not observed |
| | | | | evaluated | | |
| Kras-I_{mb1} | KrasG12D/WT Ikk2stopF/WT | 2 | mb1-cre | 105 | 20 | T-ALL |
| Kras-Blcγ1 | KrasG12D/WT BCL2Eμ Ikk2stopF/WT | 3 | cγ1-cre | 262 | 40 | Multiple myeloma |
| Trp53-Blcγ1 | Trp53F/WT BCL2Eμ Ikk2stopF/WT | 3 | cγ1-cre | 258 | 38 | Multiple myeloma |
| CyclinD1-Blcγ1 | CyclinD1Eμ BCL2Eμ lkk2stopF/WT | 3 | cγ1-cre | 328 | 43 | Multiple myeloma |
| Maf-Blcγ1 | MAFEμ BCL2Eμ lkk2stopF/WT | 3 | cγ1-cre | 331 | 31 | Multiple myeloma |
| MMSET-BIcγ1 | MMSETstopF/WT BCL2Eμ Ikk2stopF/WT | 3 | cγ1-cre | 313 | 40 | Multiple myeloma |
| Kras-MI_{cγ1} | KrasG12D/WT MYCstopF/WT Ikk2stopF/WT | 3 | cγ1-cre | 57 | 21 | Multiple myeloma |
| Trp53-MIcγ1 | Trp53F/WT MYCstopF/WT Ikk2stopF/WT | 3 | cγ1-cre | 138 | 14 | Multiple myeloma |
| Maf-Ml_{cγ1} | MAFEμ MYCstopF/WT Ikk2stopF/WT | 3 | cγ1-cre | 205 | 33 | Multiple myeloma |
| B-MI_{cγ1} | BCL2Eμ MYCstopF/WT Ikk2stopF/WT | 3 | cγ1-cre | 108 | 9 | Multiple myeloma |
| Trp53/Kras-Icγ1 | KrasG12D/WT Trp53F/WT Ikk2stopF/WT | 3 | cγ1-cre | 169 | 22 | B-cell lymphoma/T-ALL |
| Trp53/Kras-Ihomcγ1 | KrasG12D/WT Trp53F/WT Ikk2stopF/stopF | 3 | cγ1-cre | 179 | 8 | B-cell lymphoma/T-ALL |
| Trp53hom/Kras- | KrasG12D/WT Trp53F/F Ikk2stopF/stopF | 3 | cγ1-cre | 130 | 47 | Plasmablastic lymphoma |
| Ihomcγ1 | | | | | | |
| KrasTrp53-Imb1 | KrasG12D/WT Trp53F/WT Ikk2stopF/WT | 3 | mb1-cre | 88 | 23 | T-ALL |
| Kras/Trp53-Blcγ1 | KrasG12D/WT Trp53F/WT BCL2Eμ | 4 | cγ1-cre | 145 | 6 | Multiple myeloma |
| | Ikk2stopF/WT | | | | | |

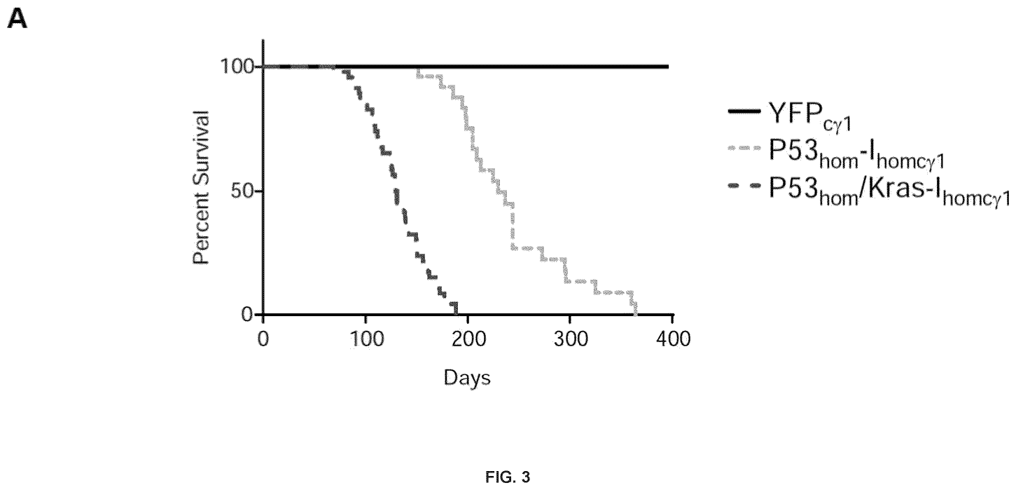

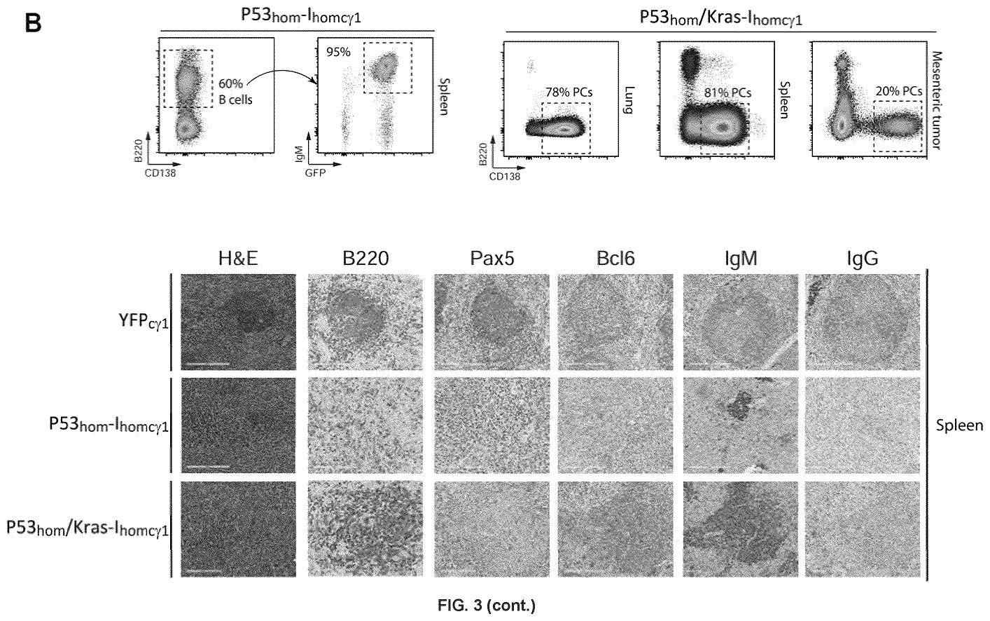

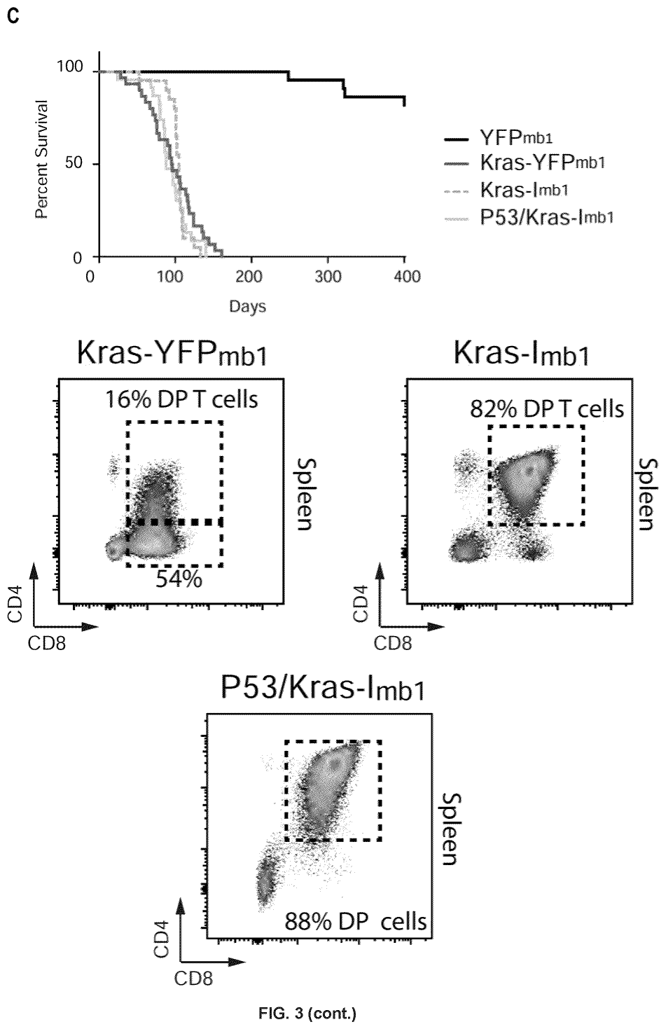

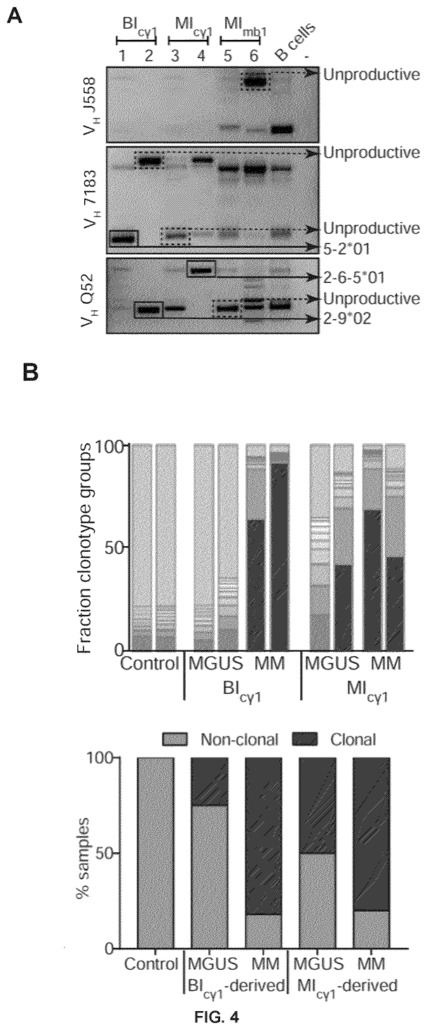

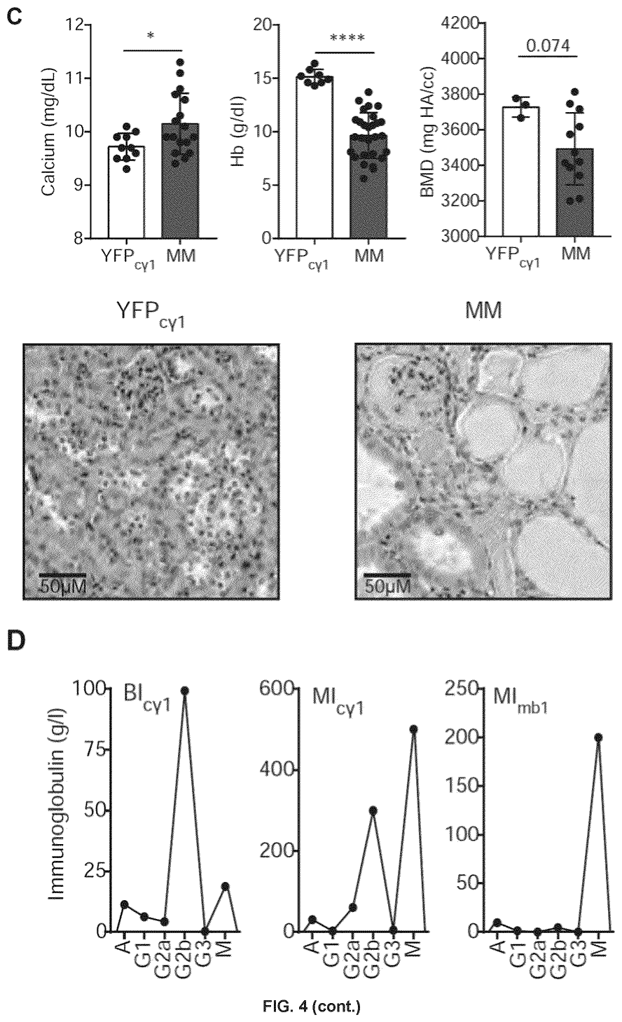

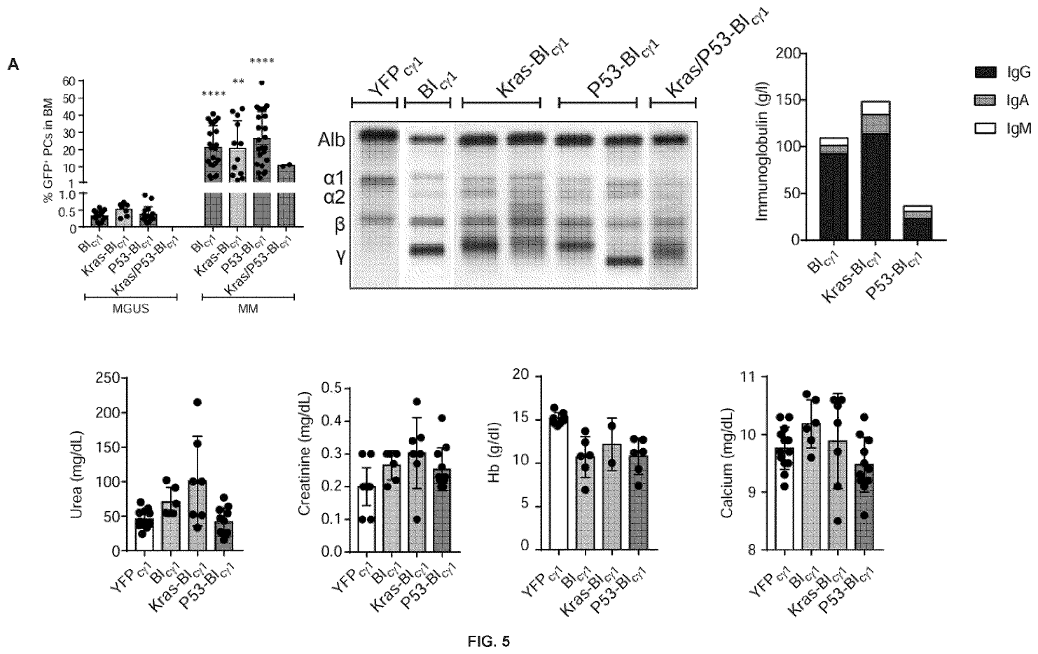

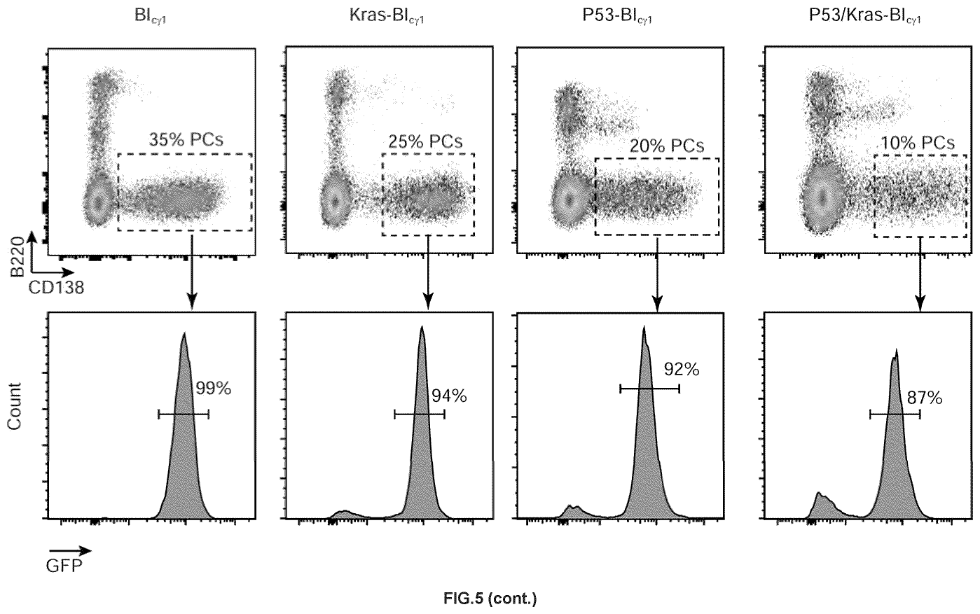

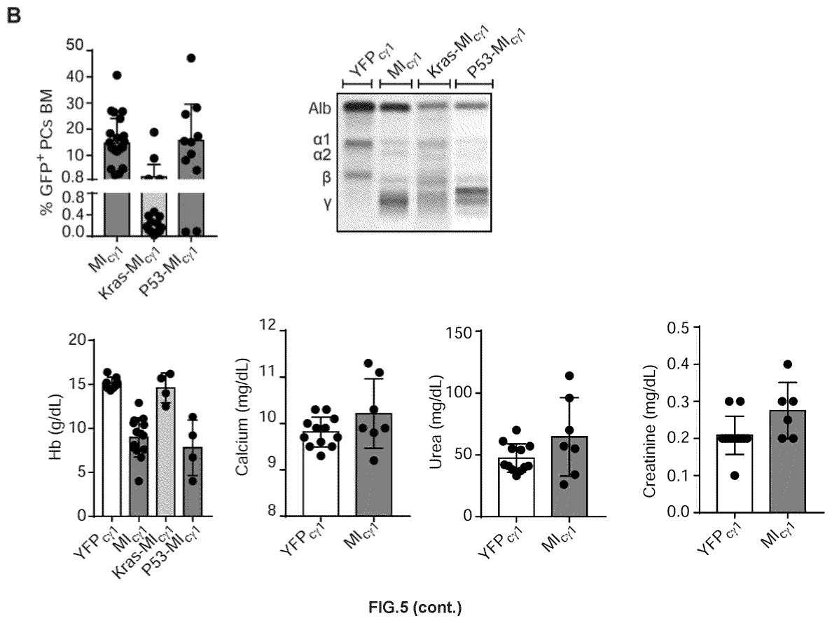

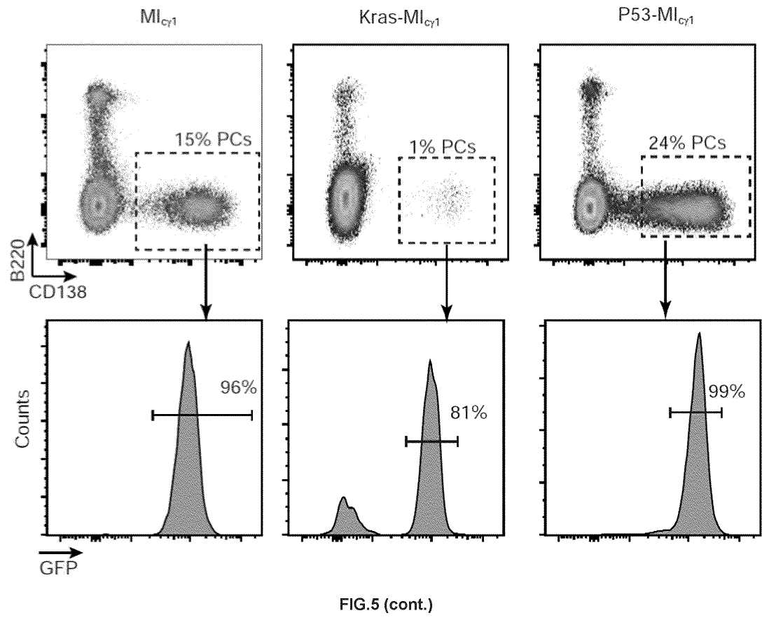

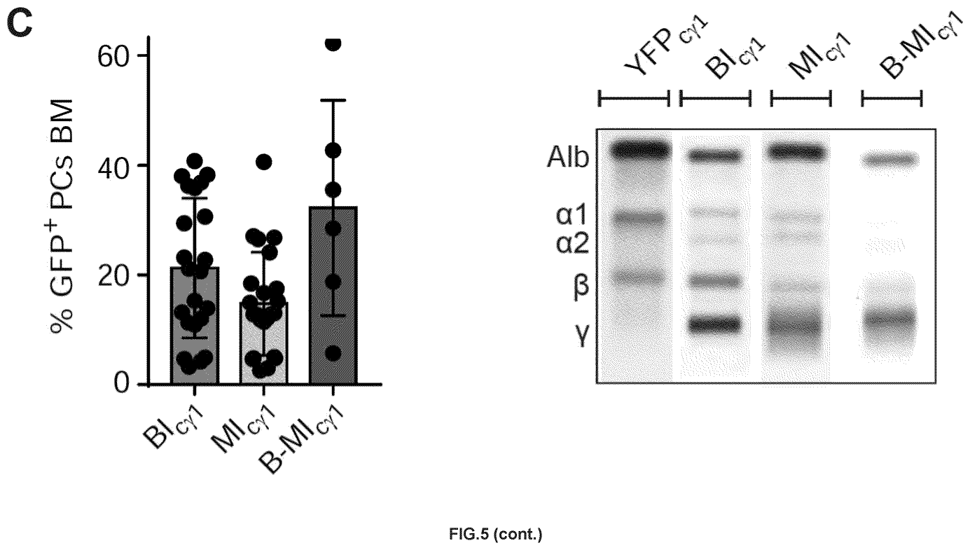

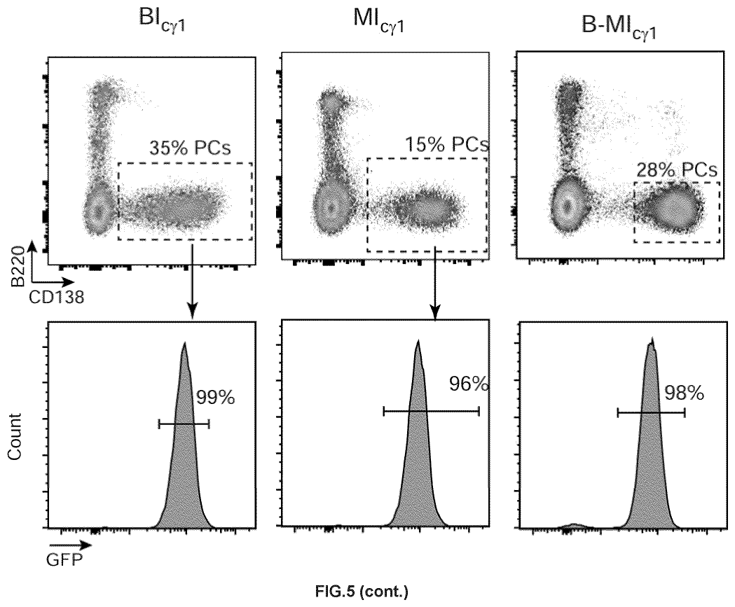

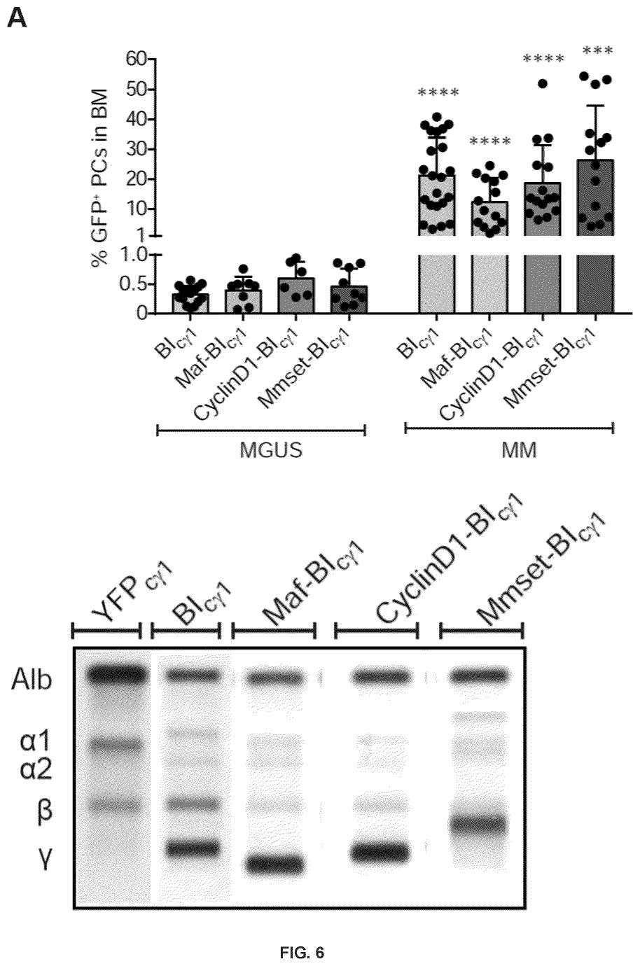

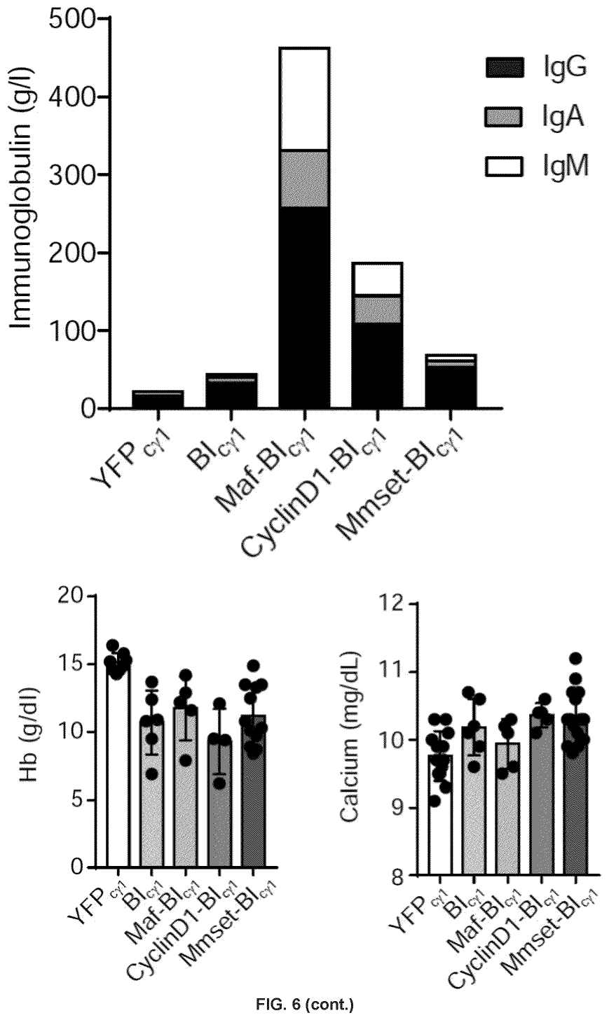

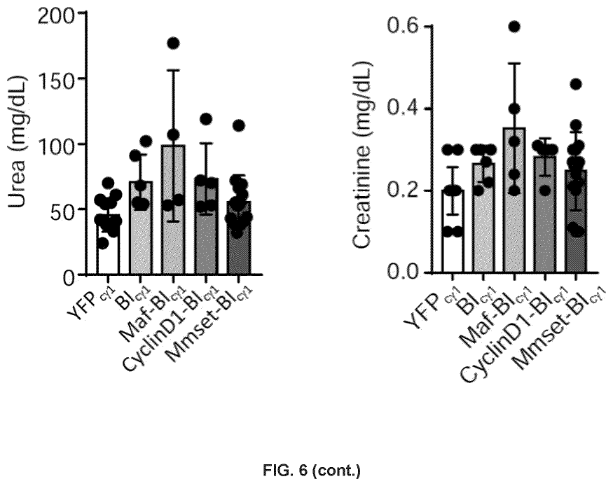

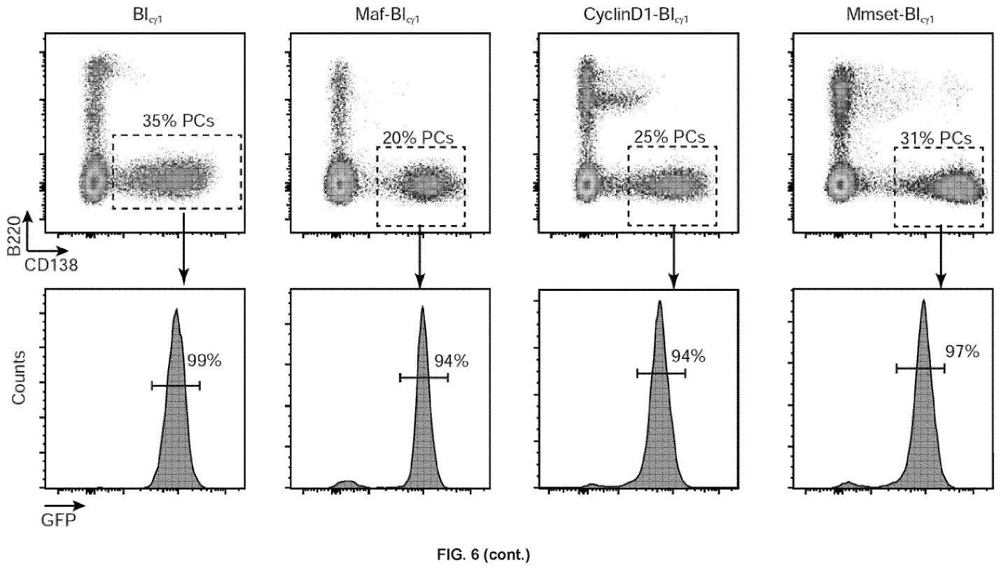

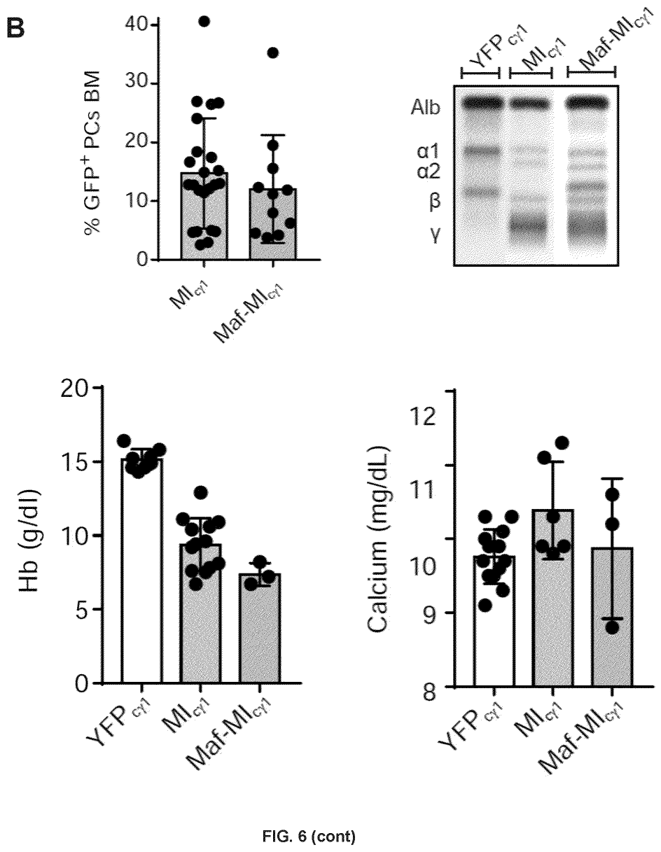

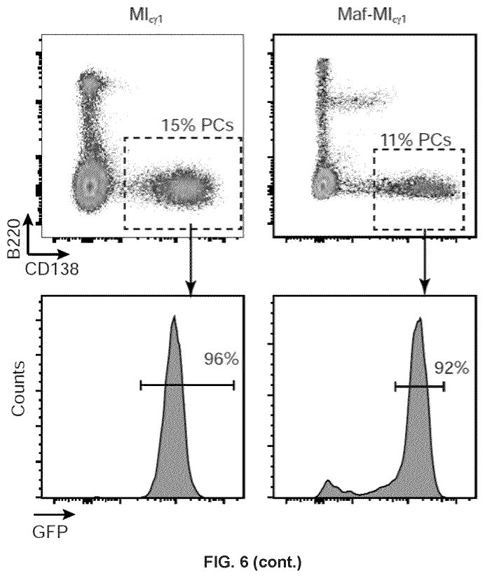

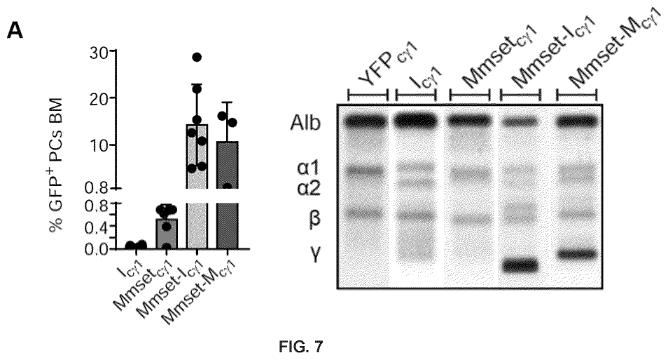

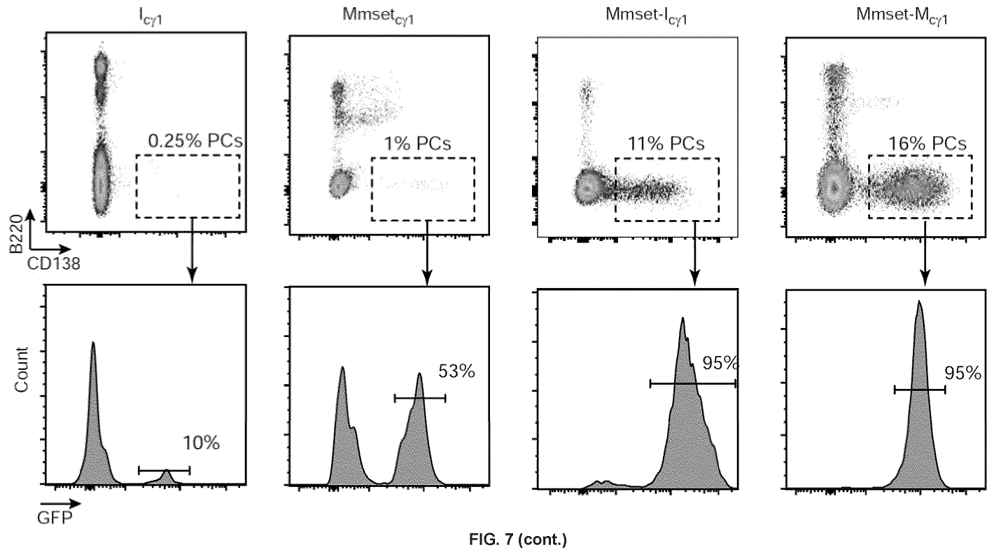

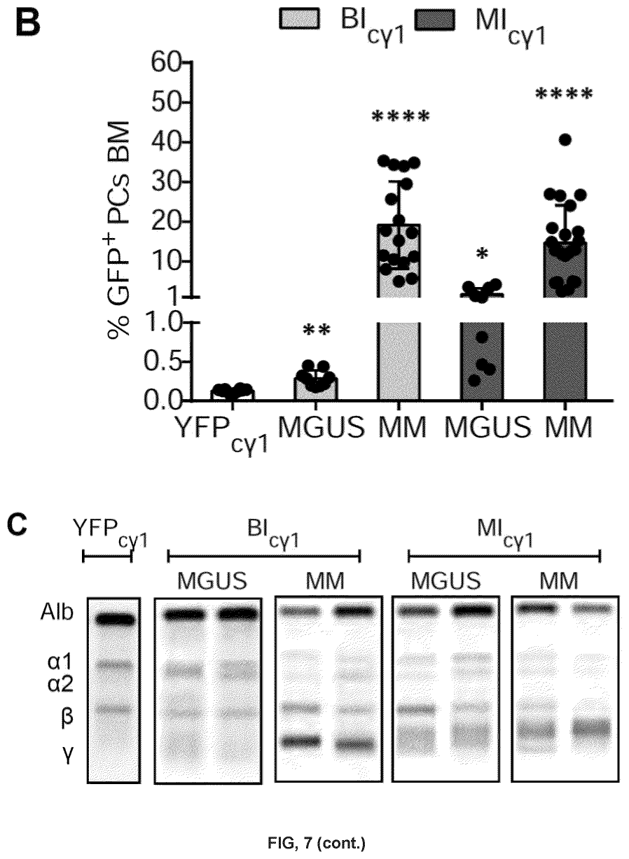

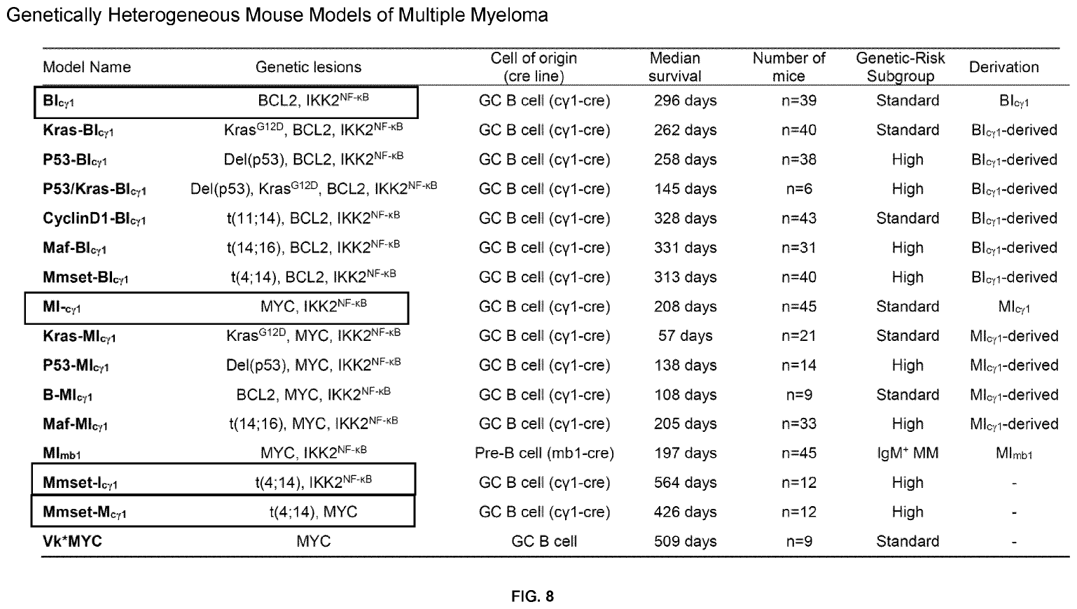

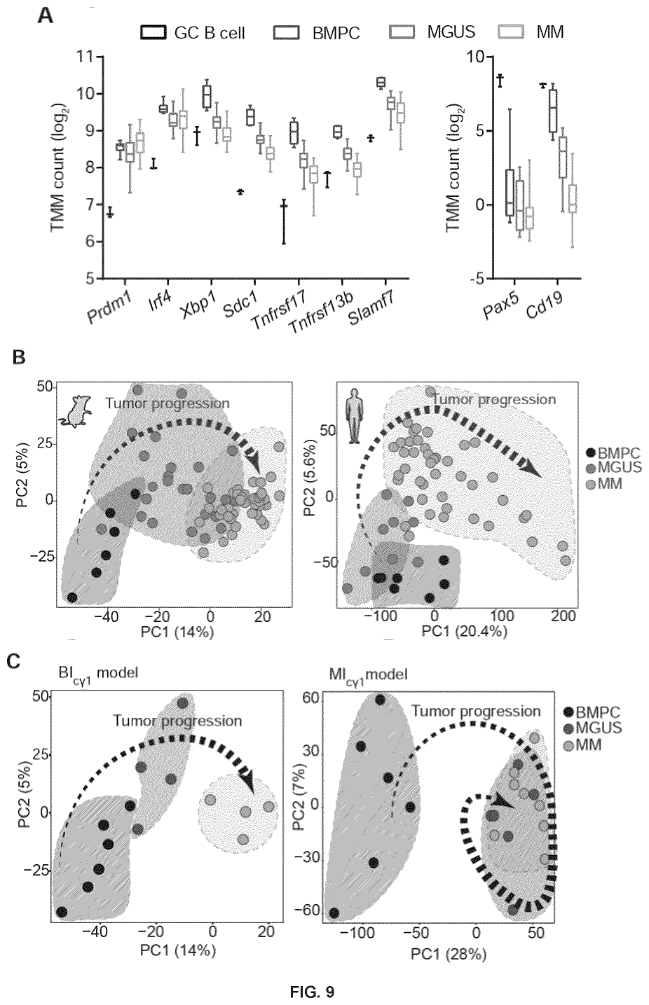

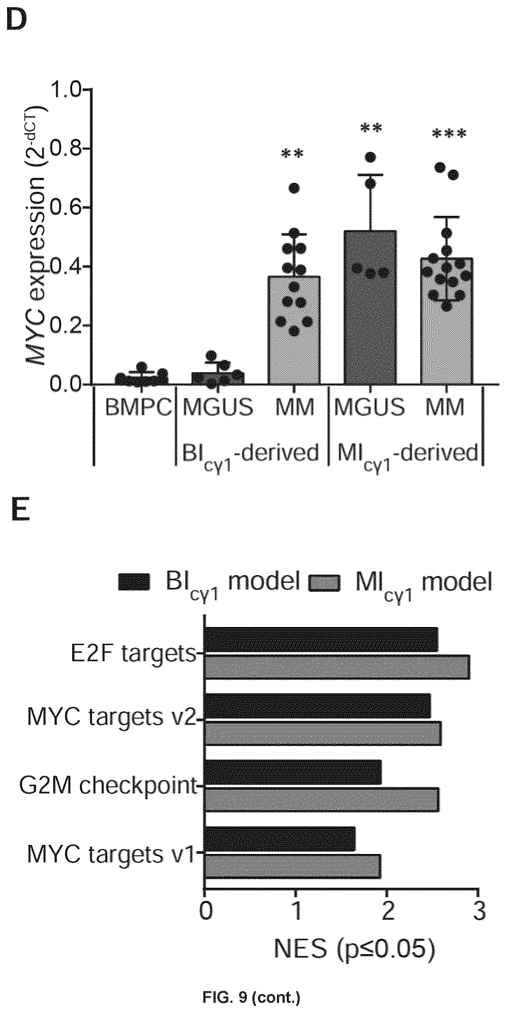

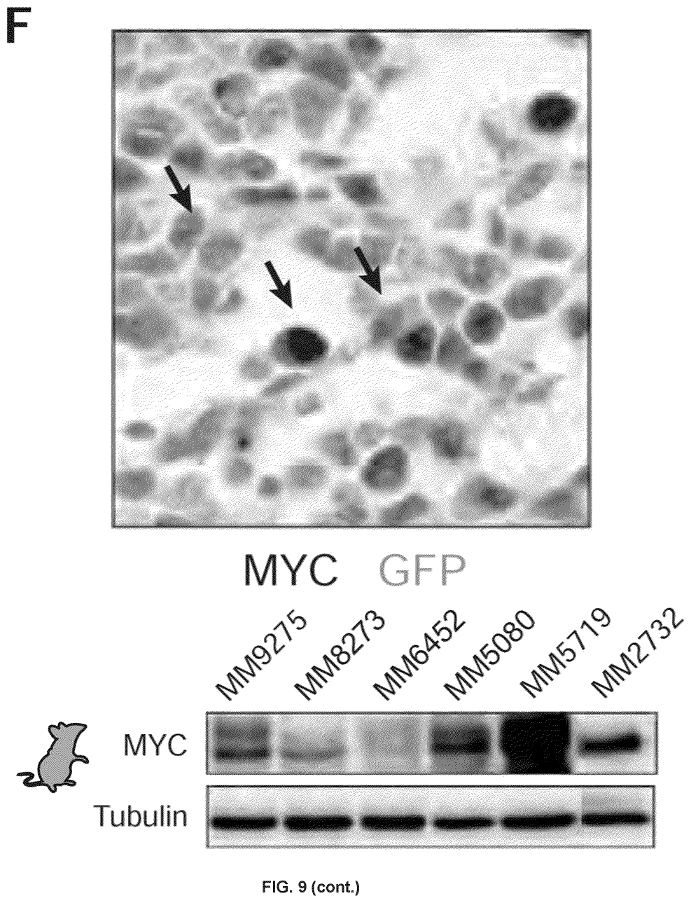

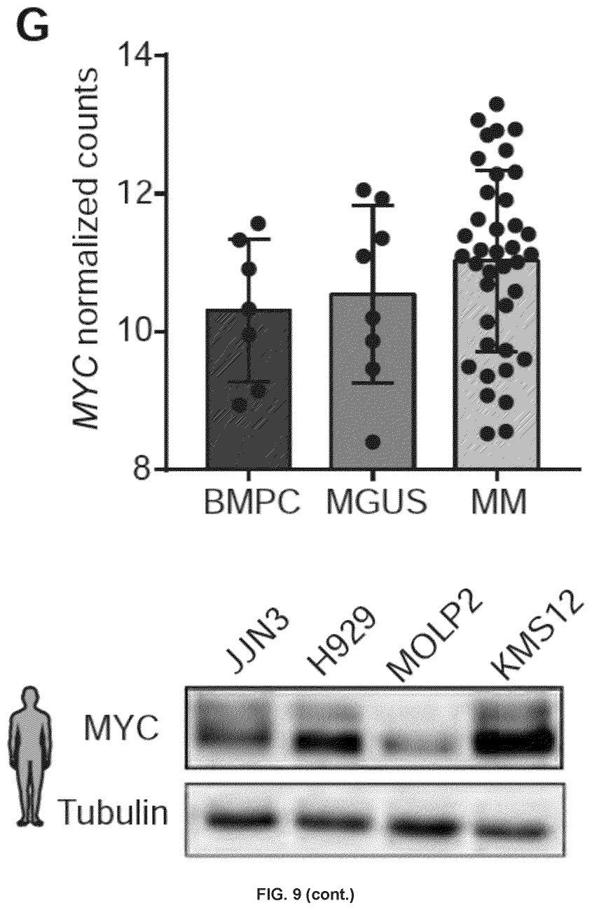

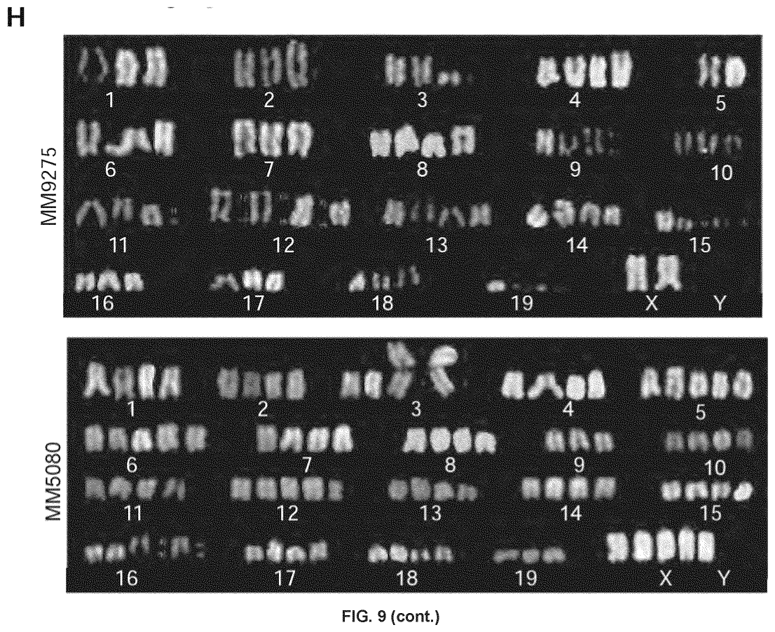

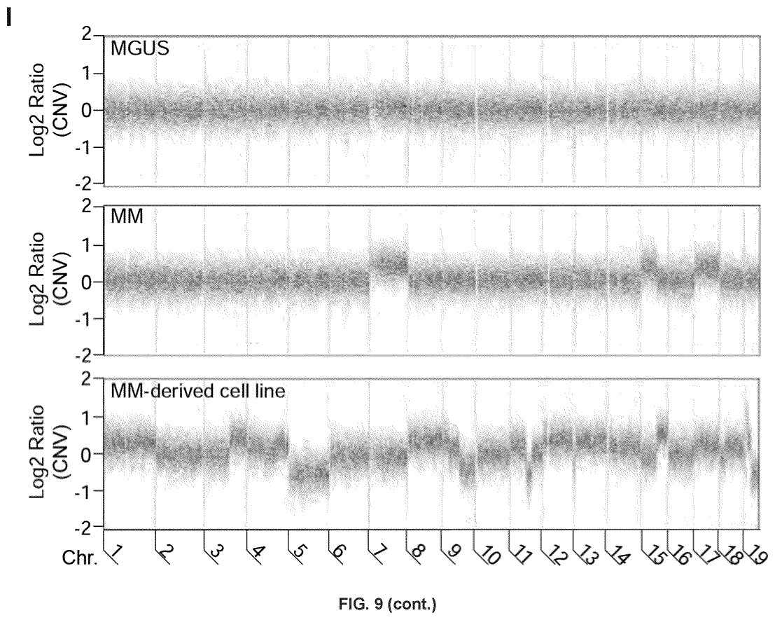

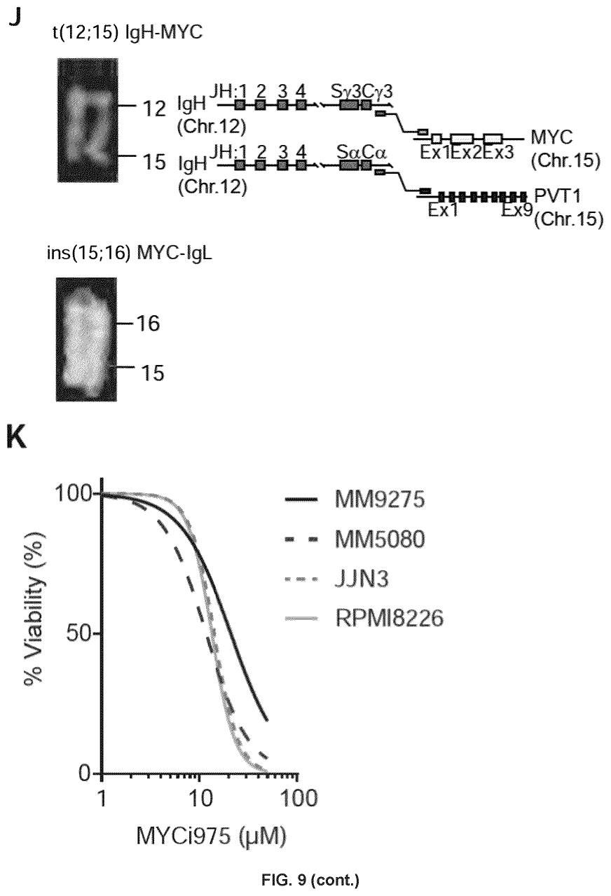

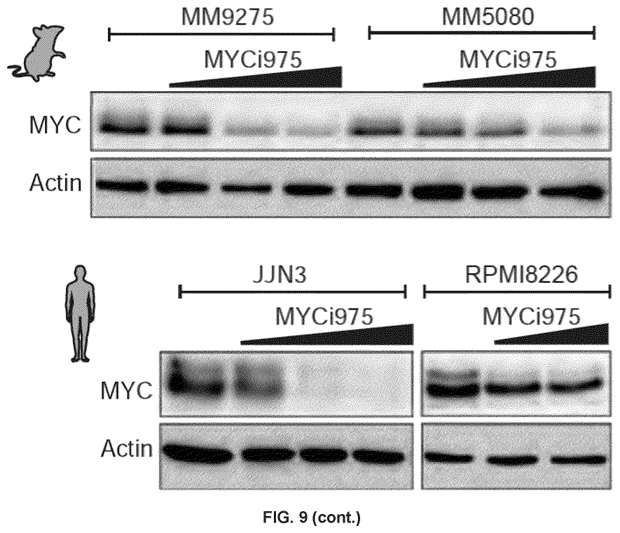

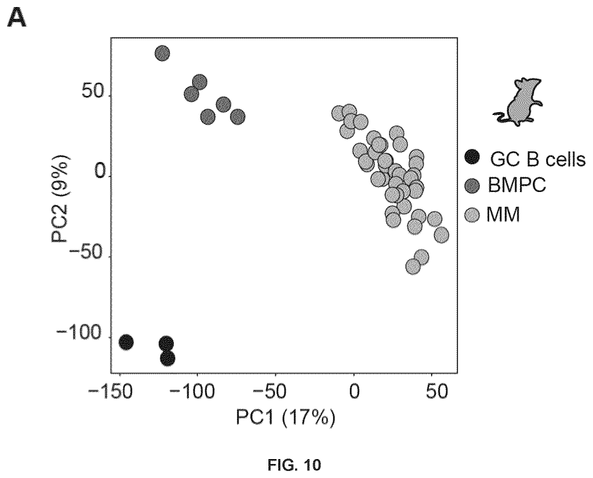

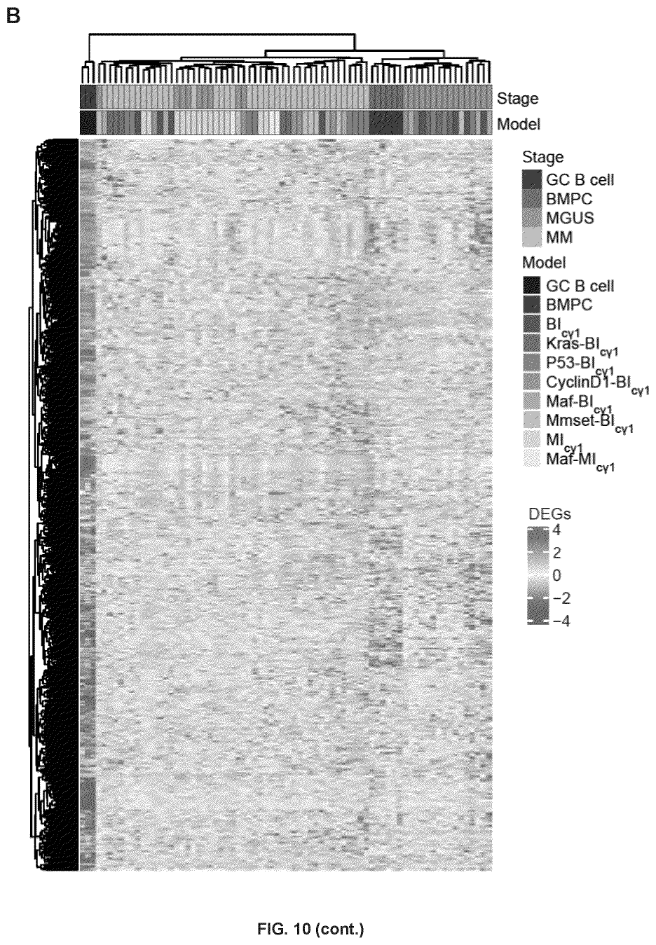

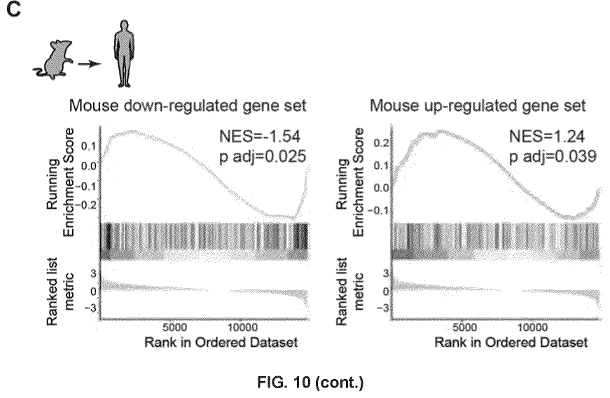

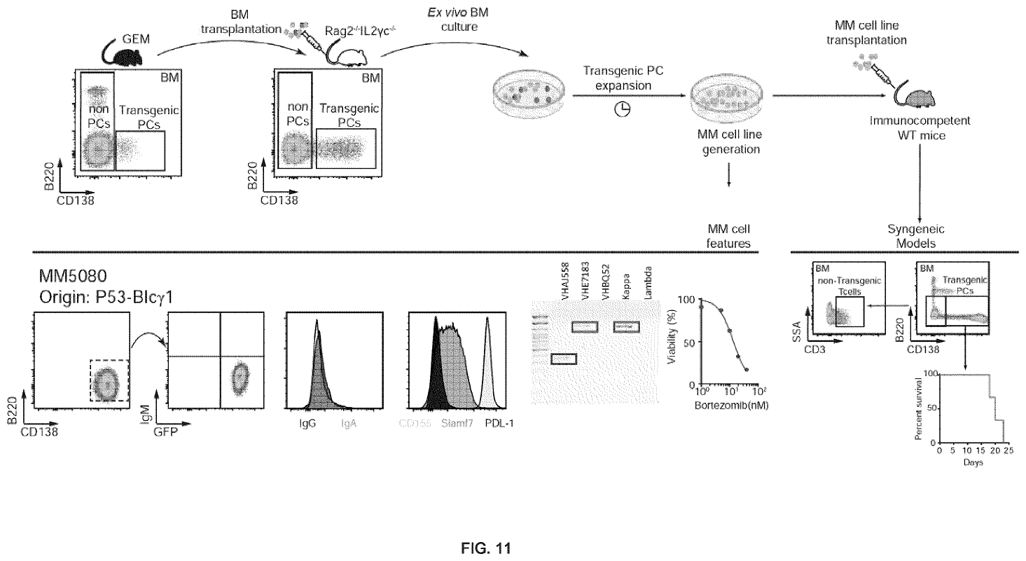

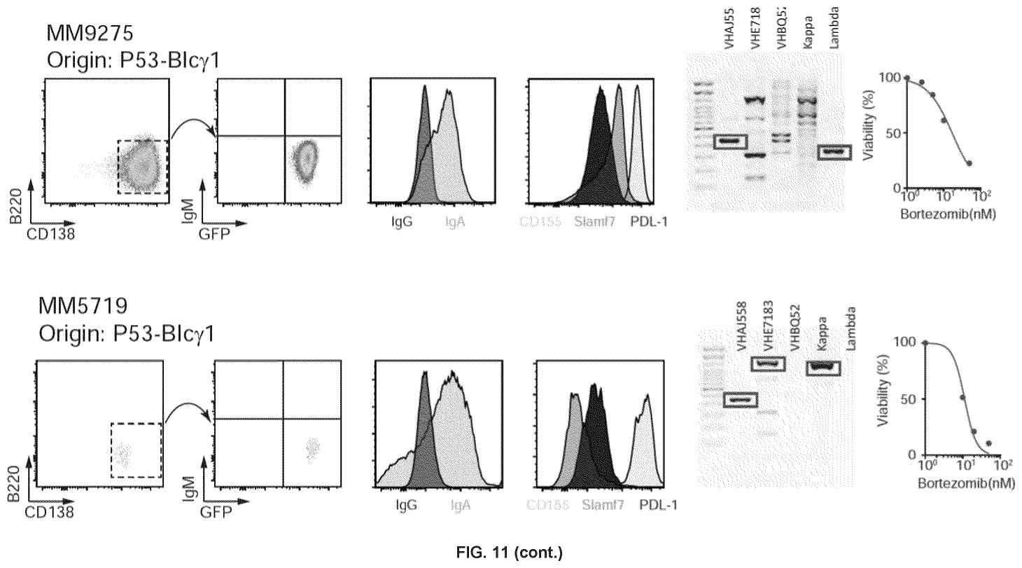

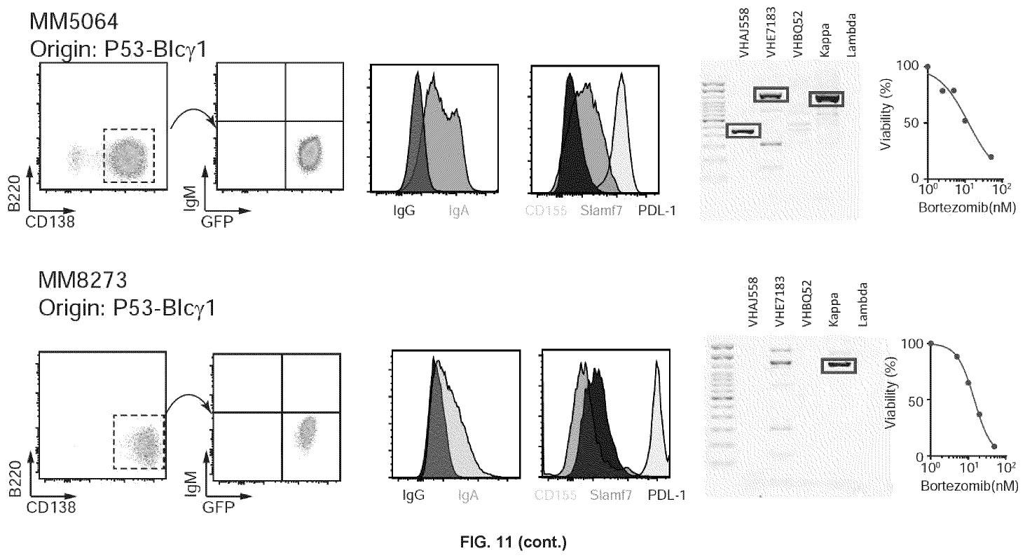

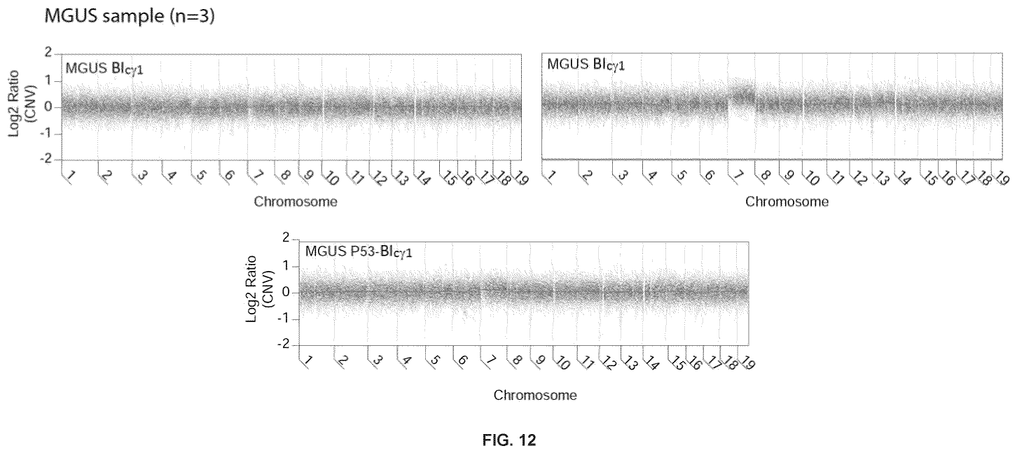

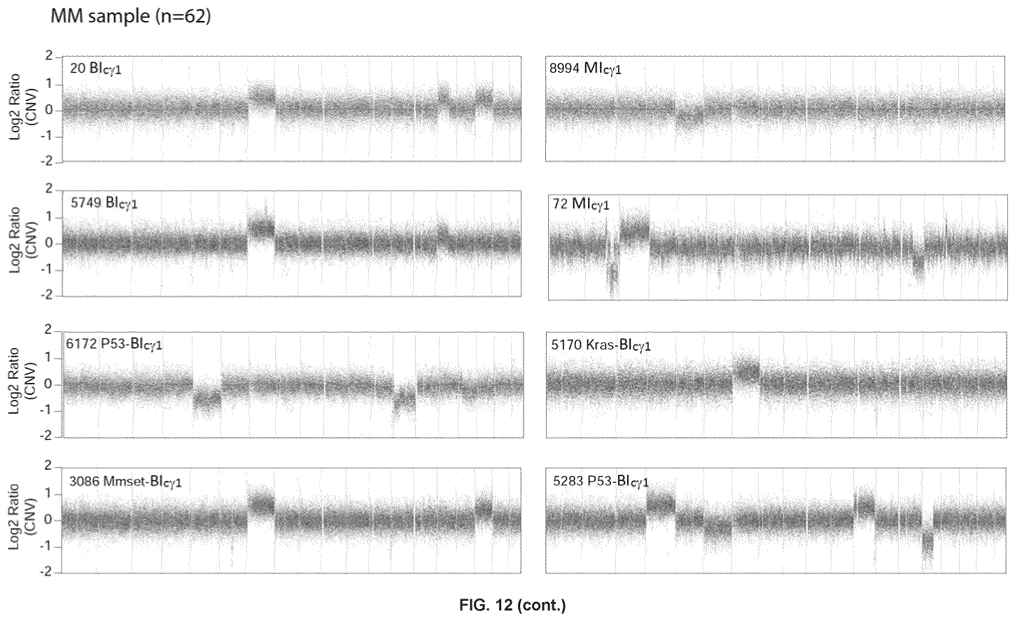

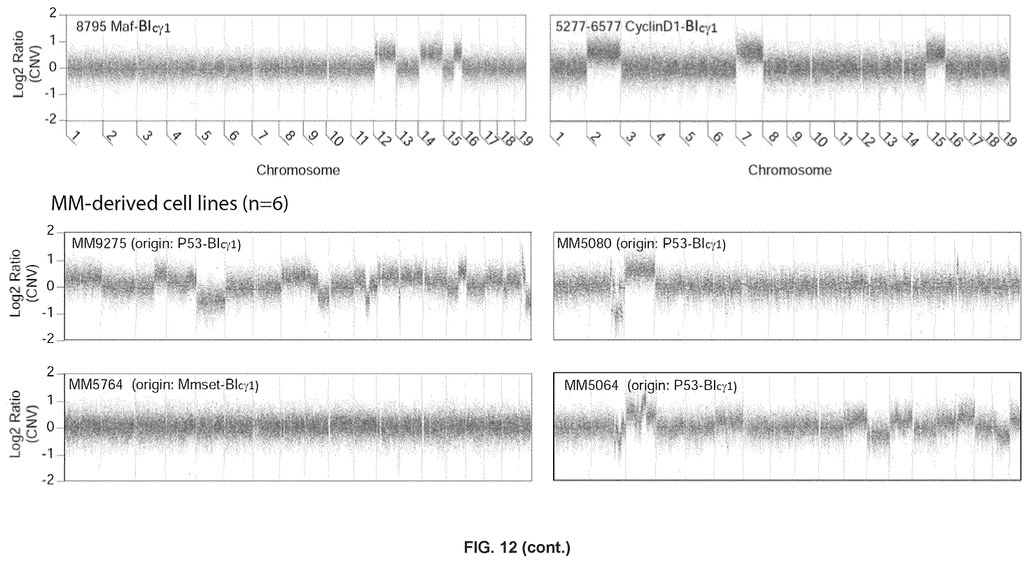

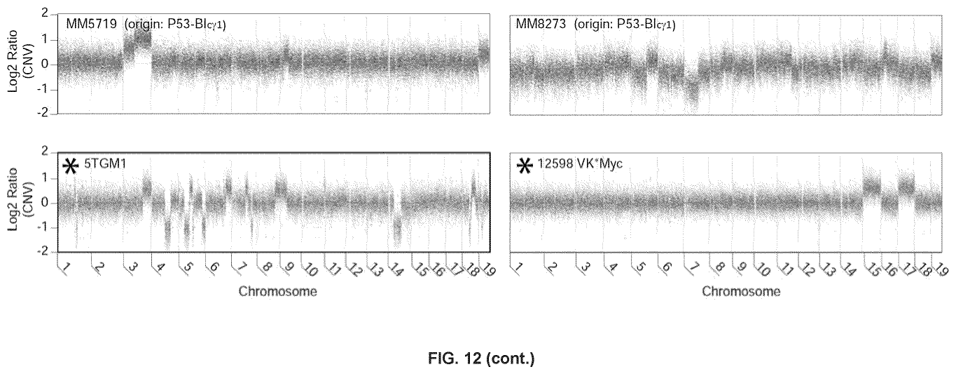

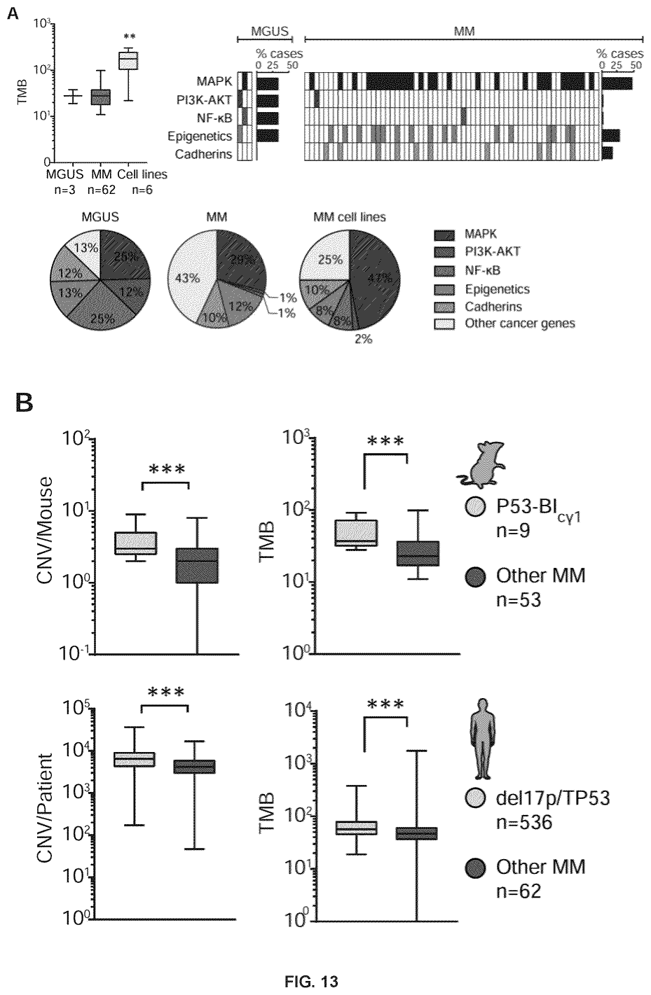

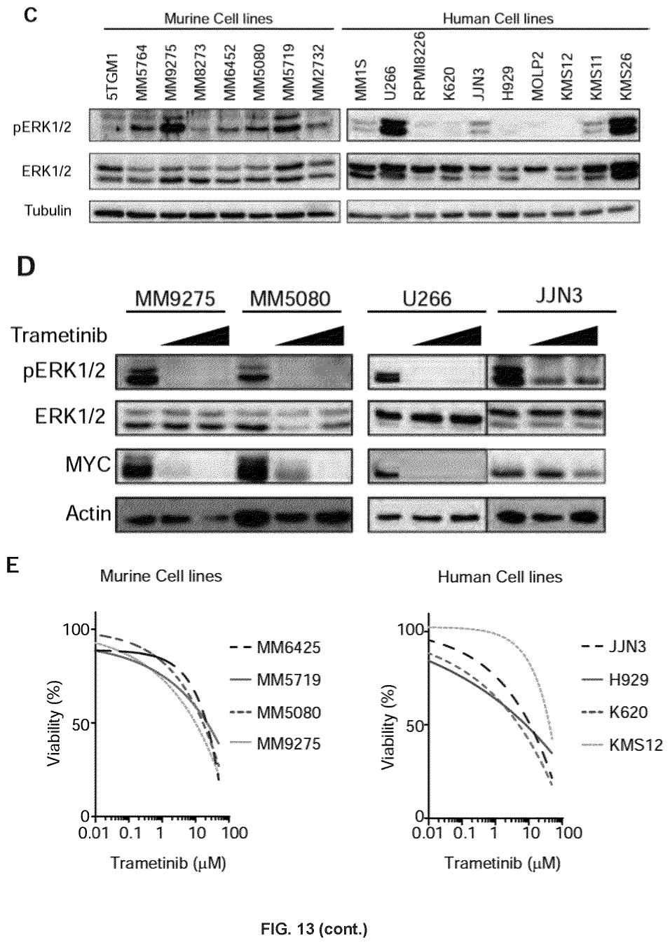

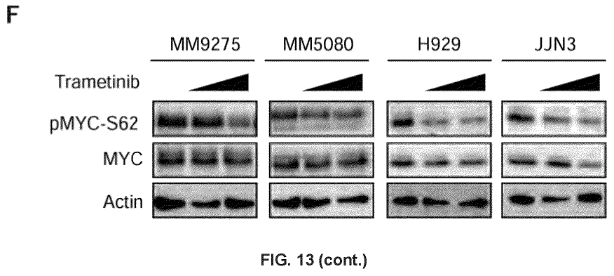

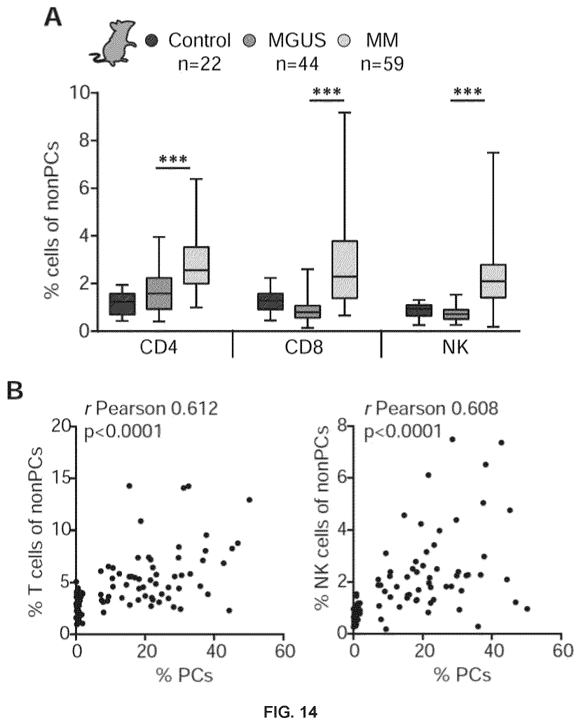

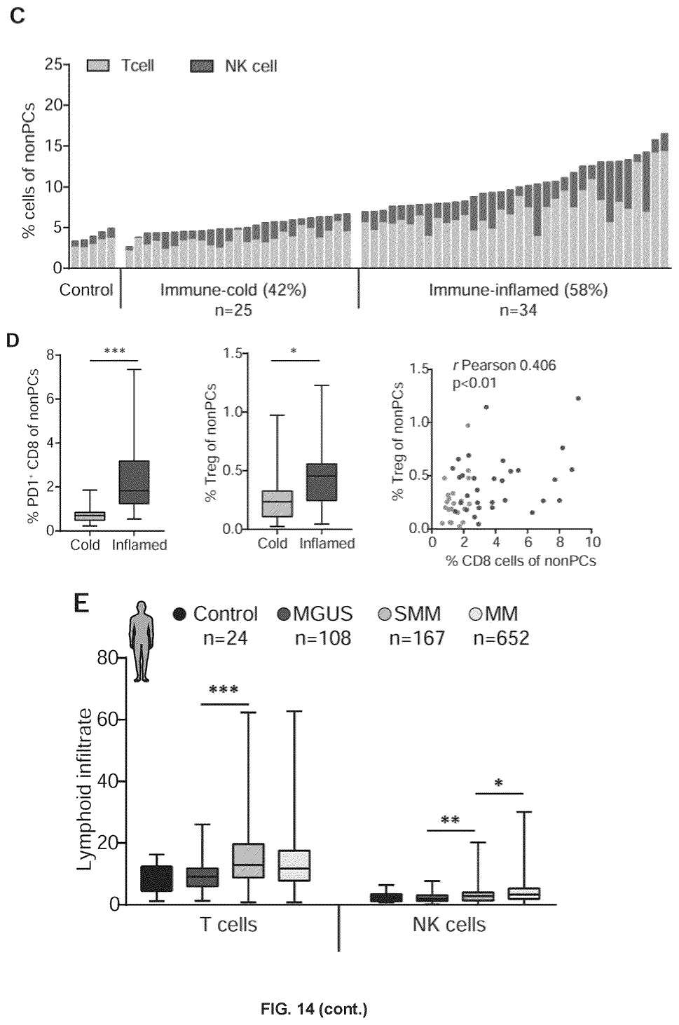

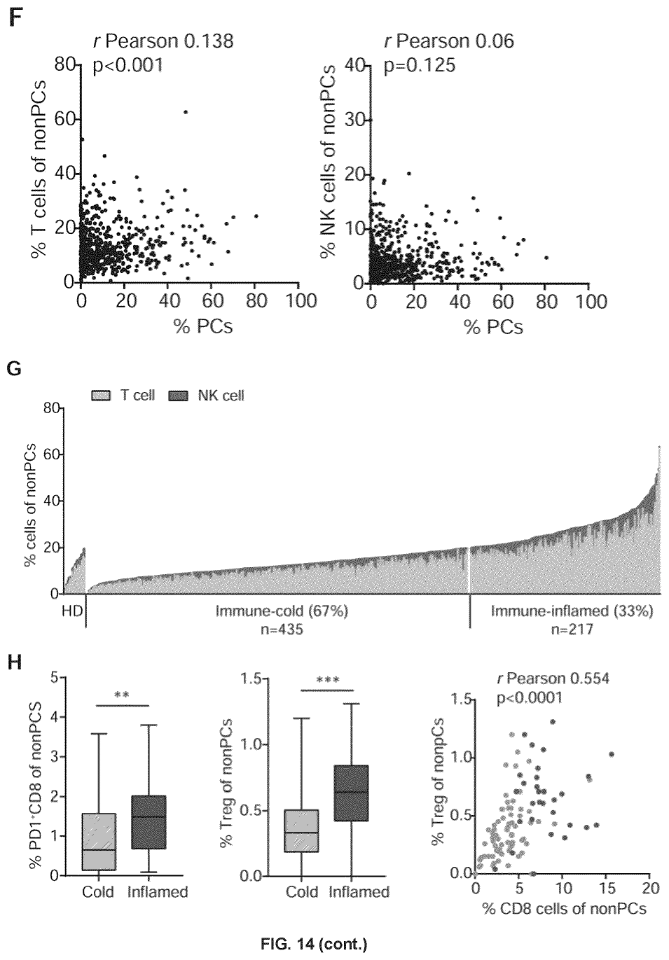

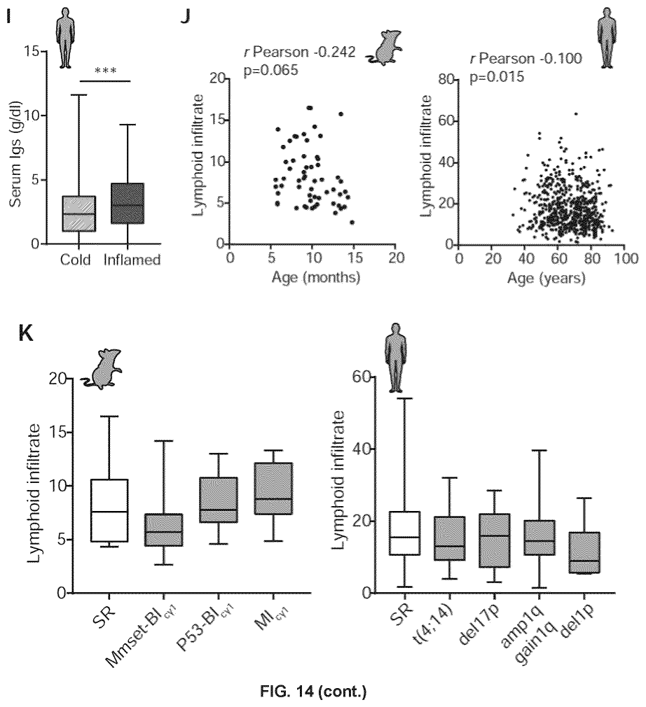

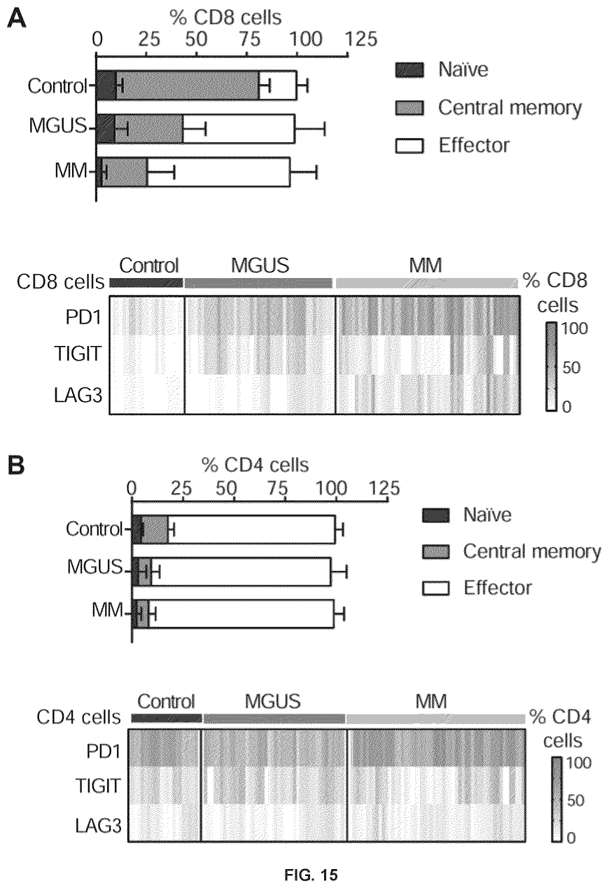

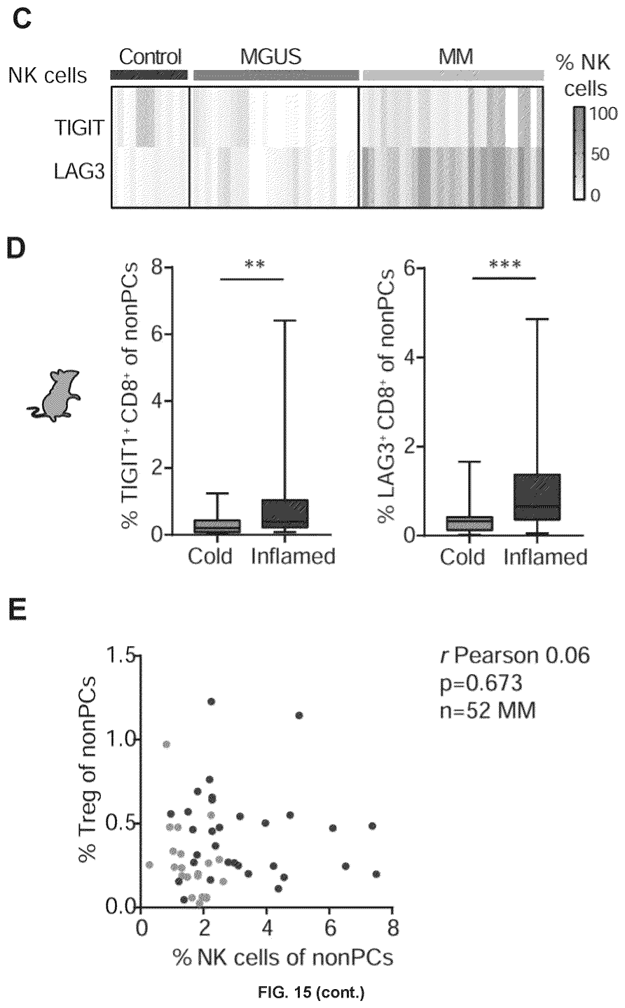

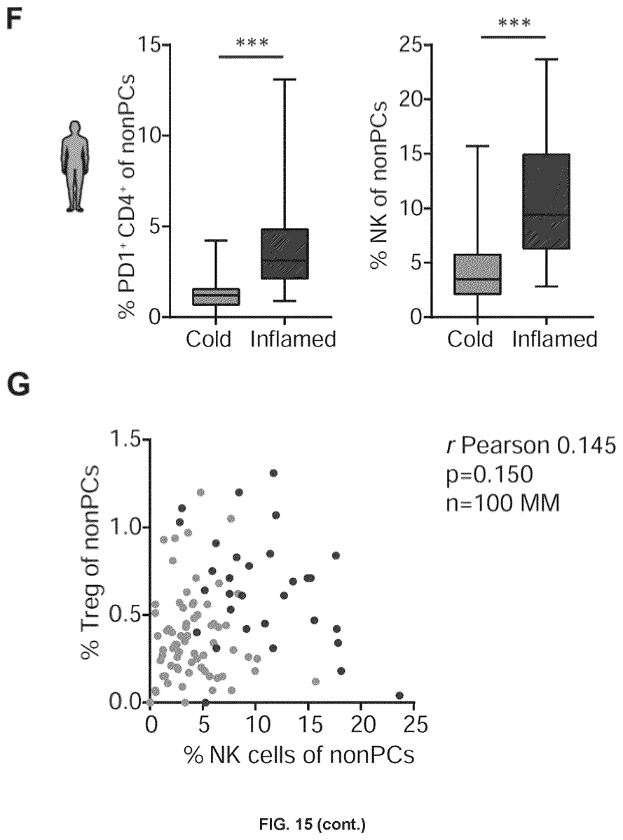

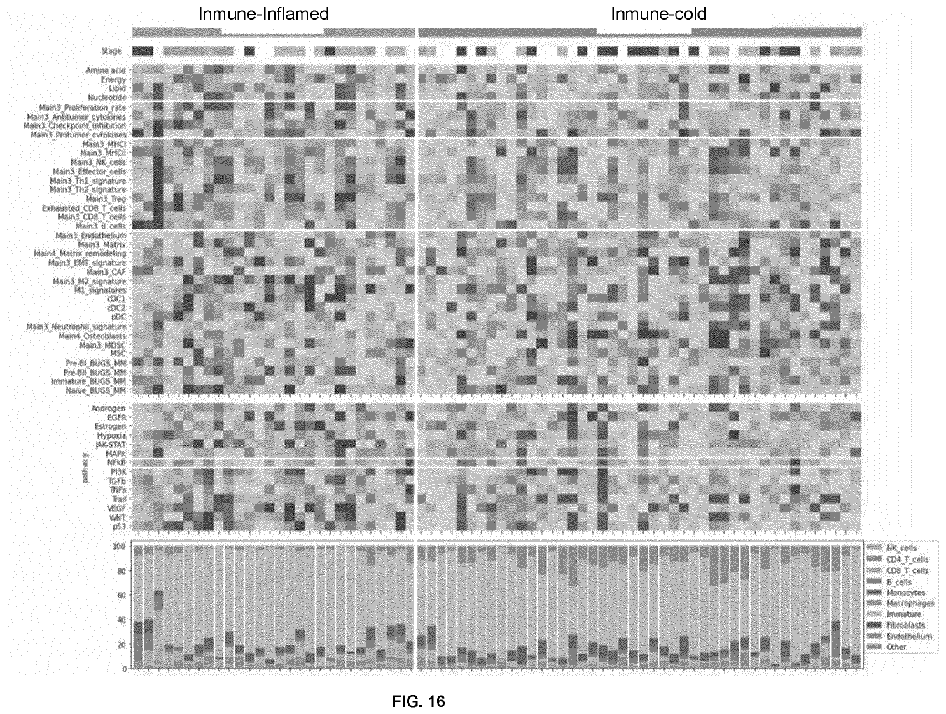

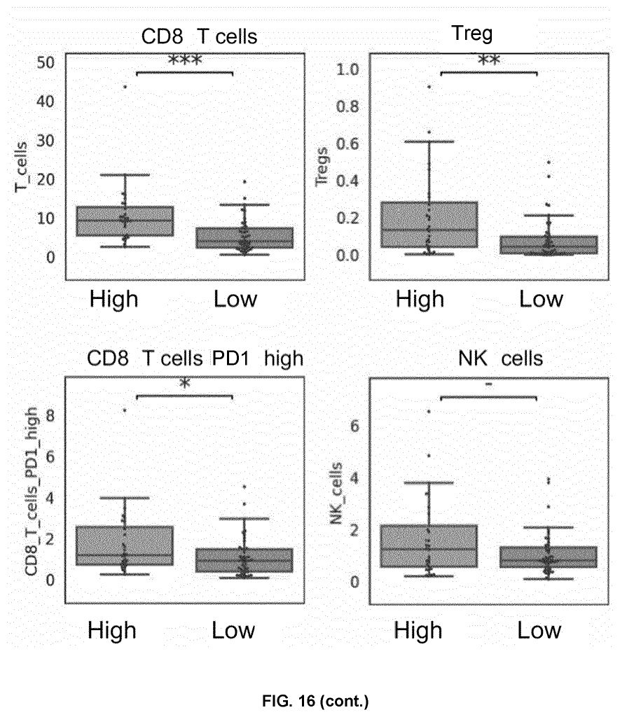

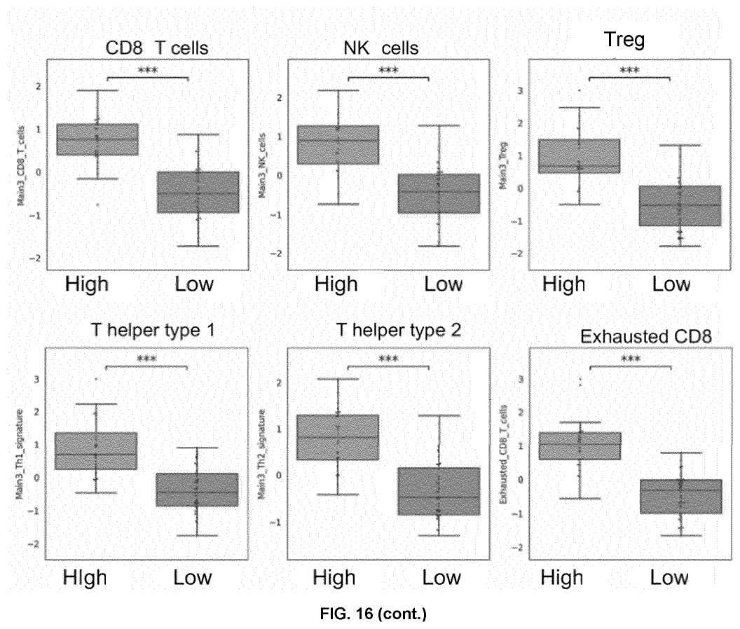

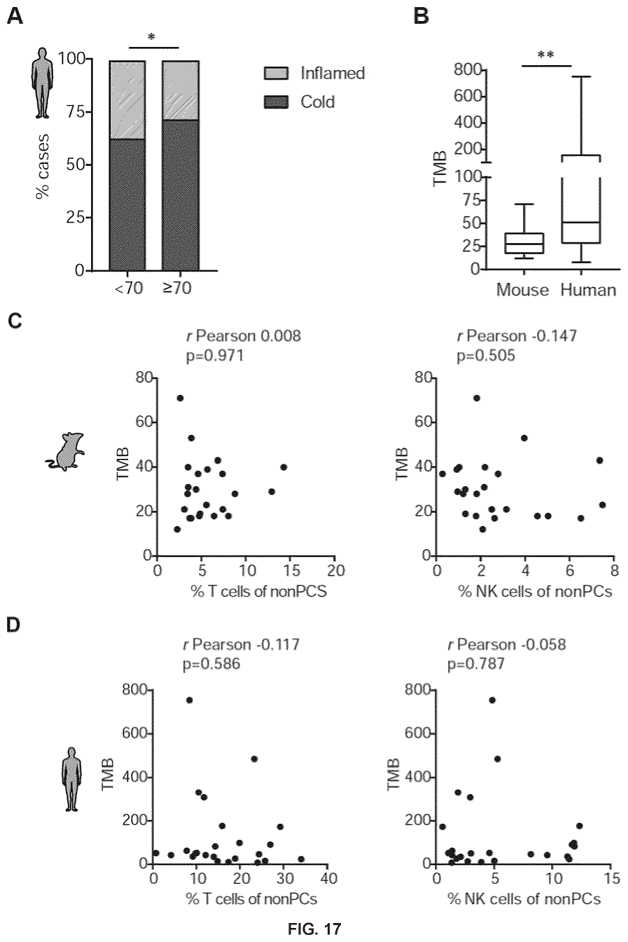

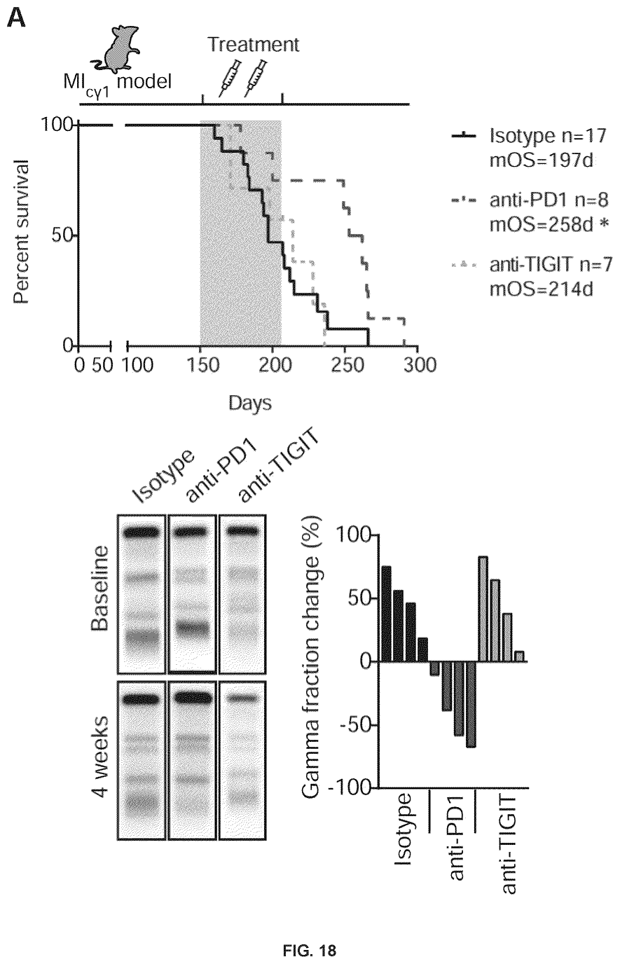

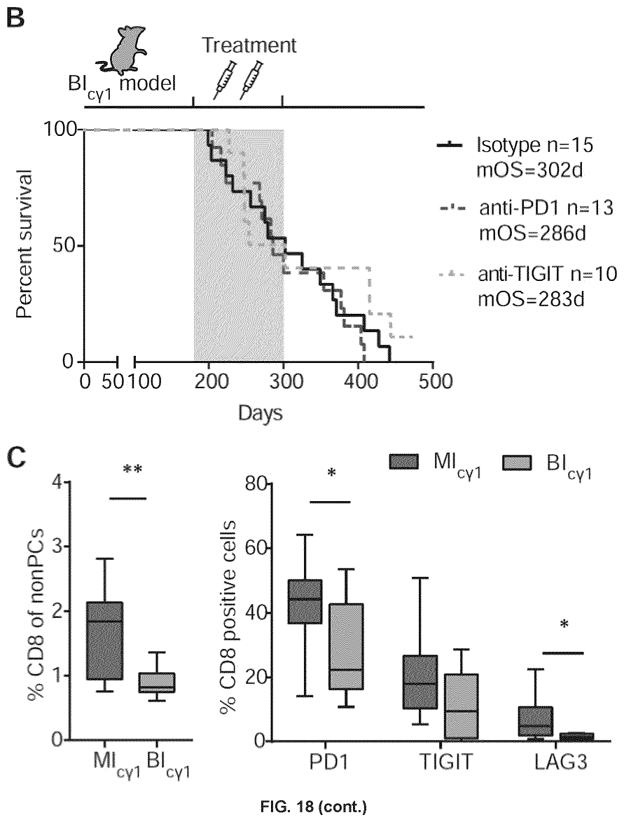

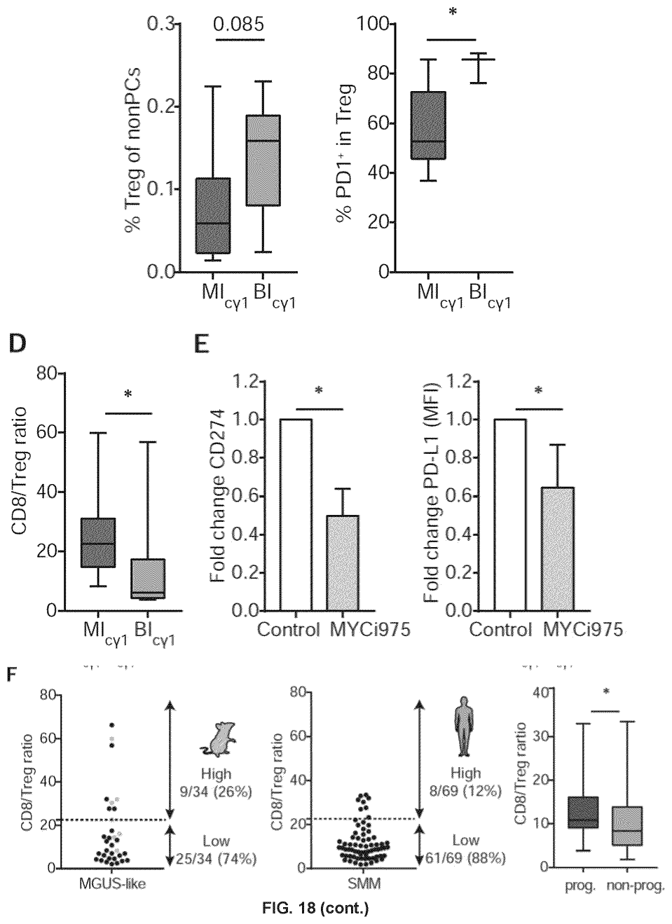

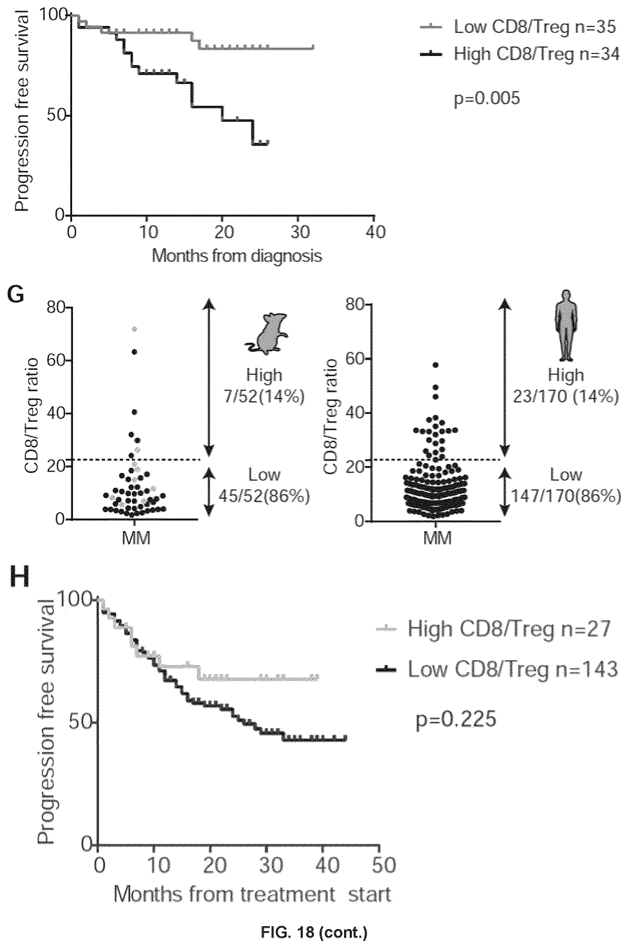

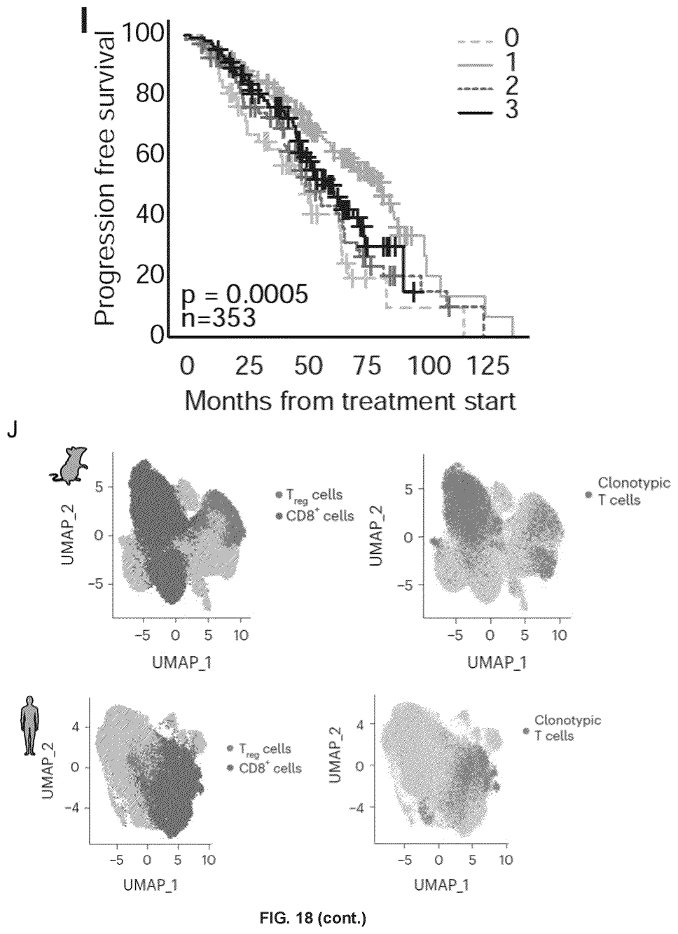

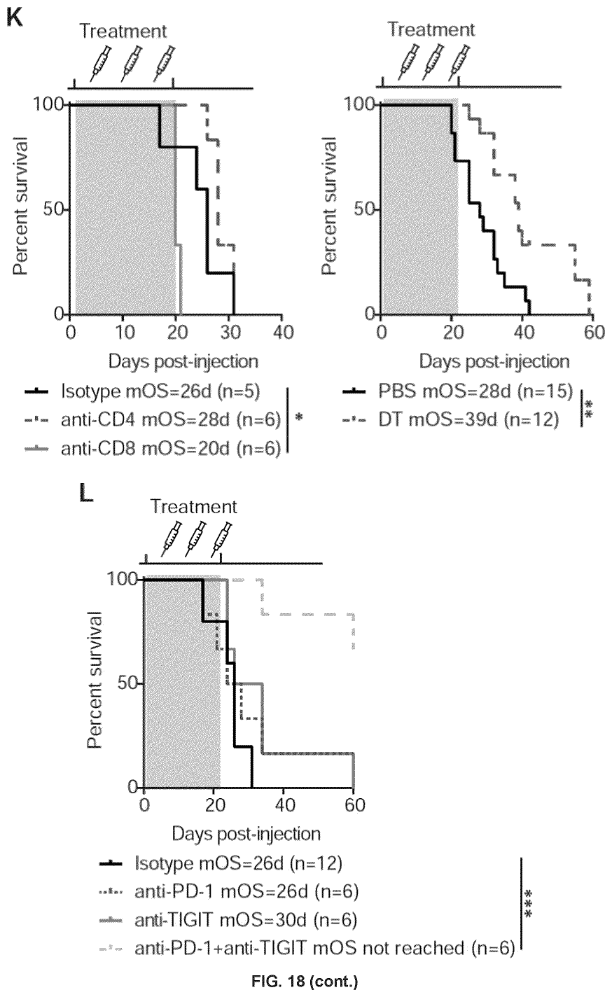

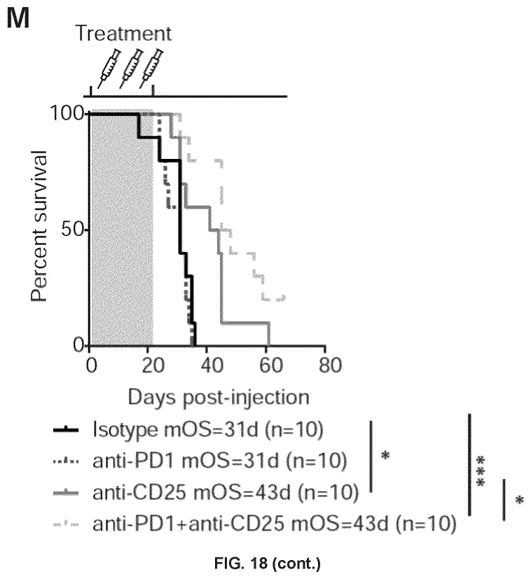

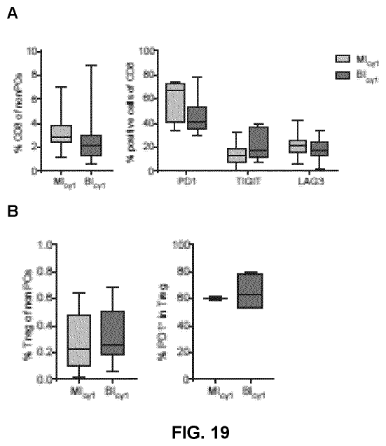

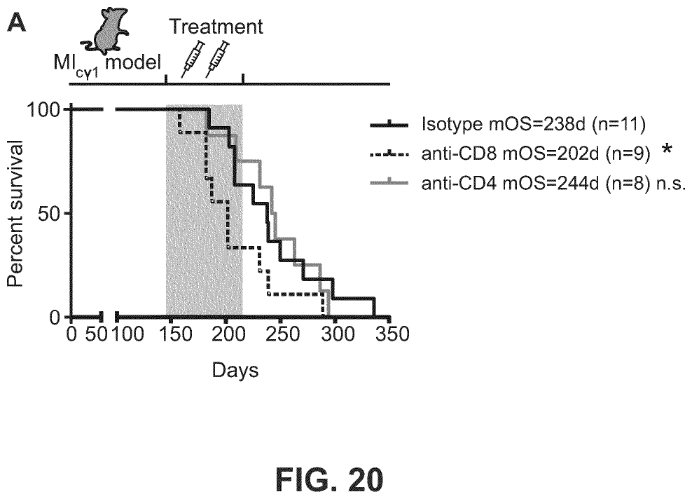

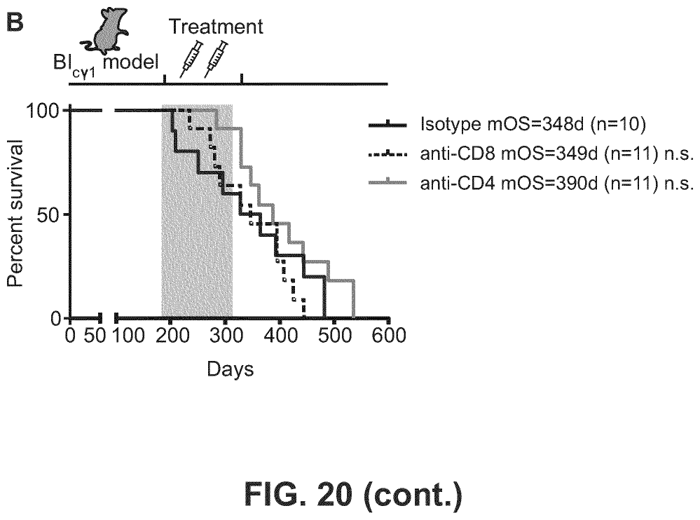

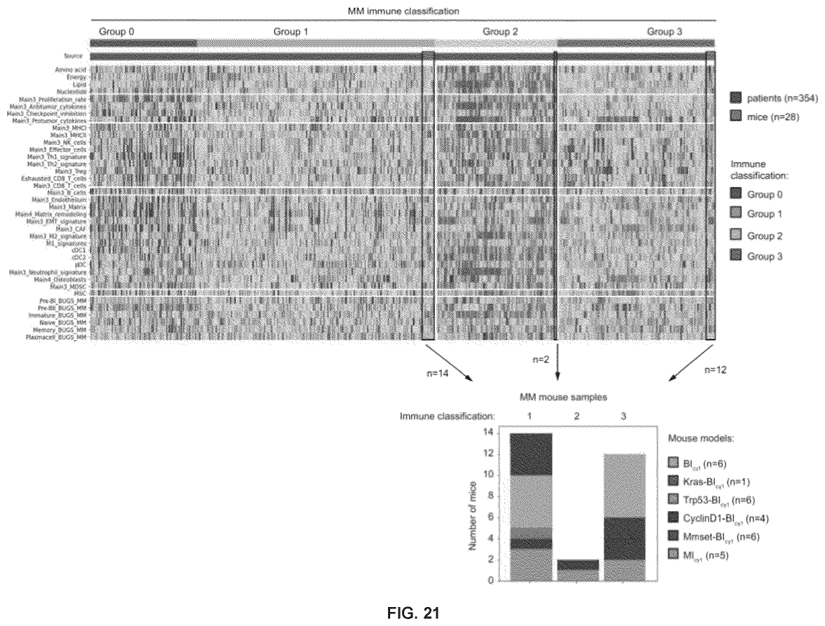

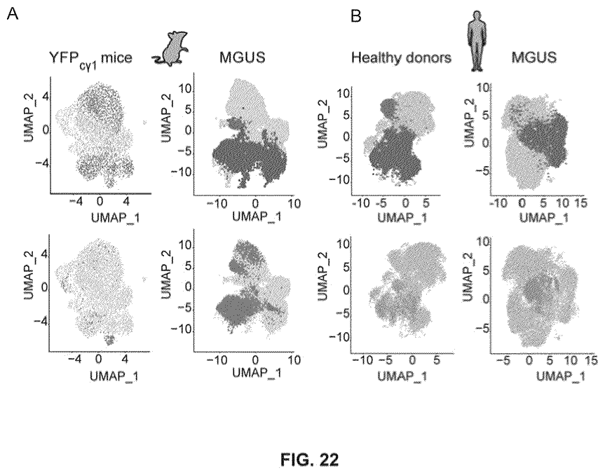

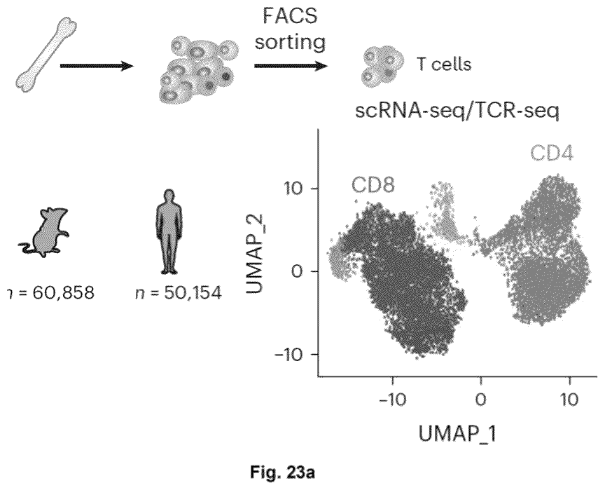

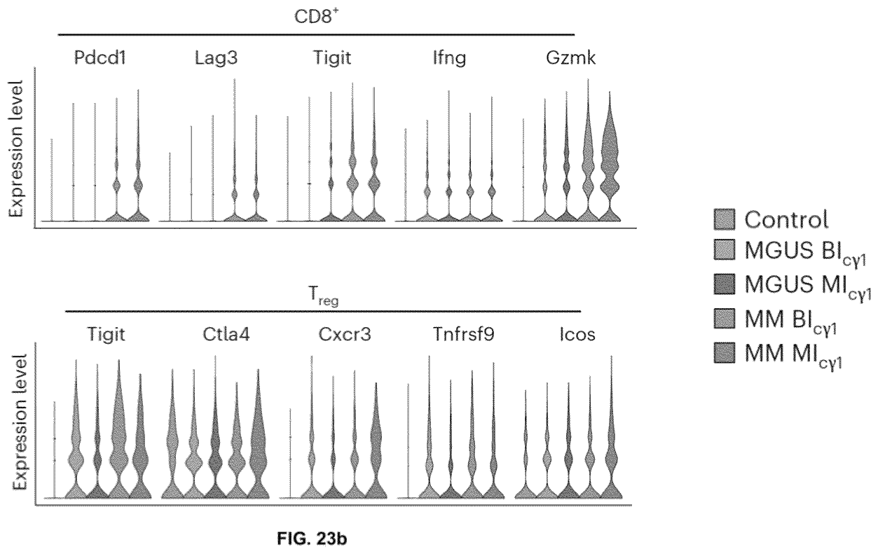

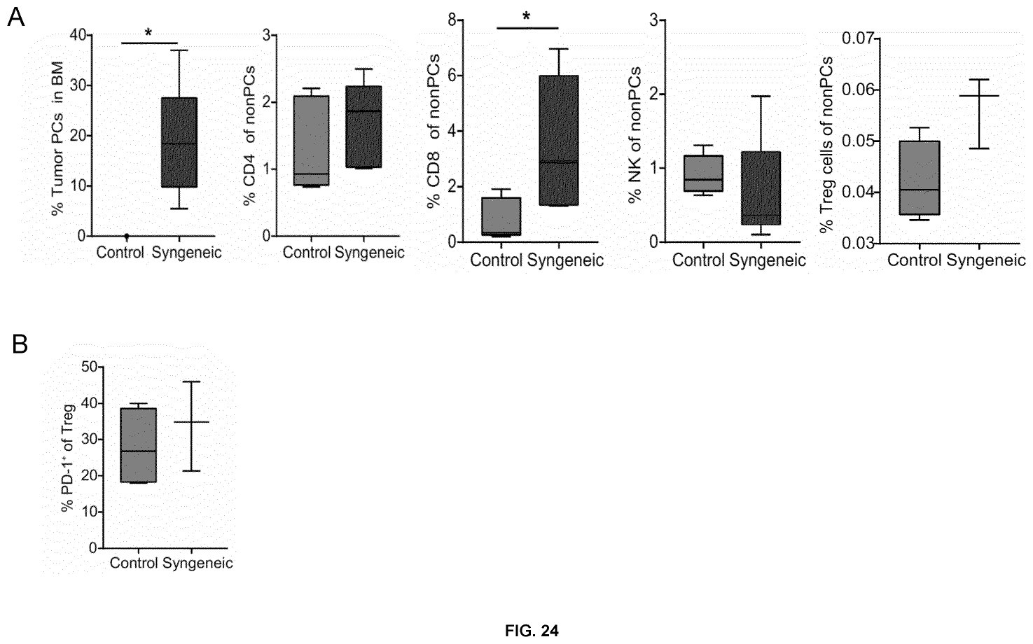

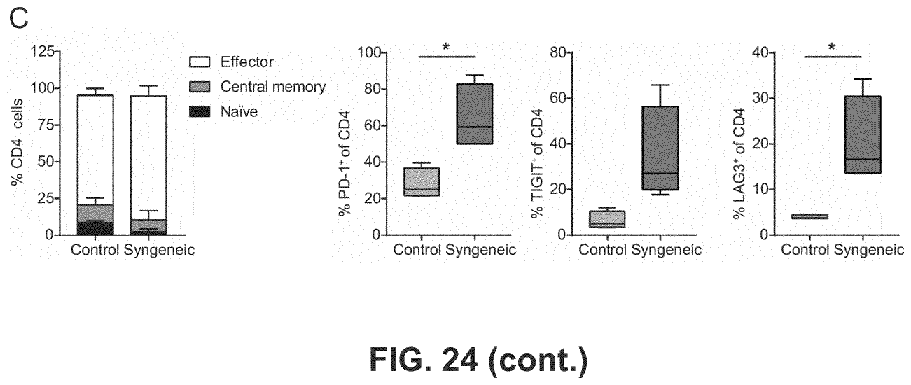

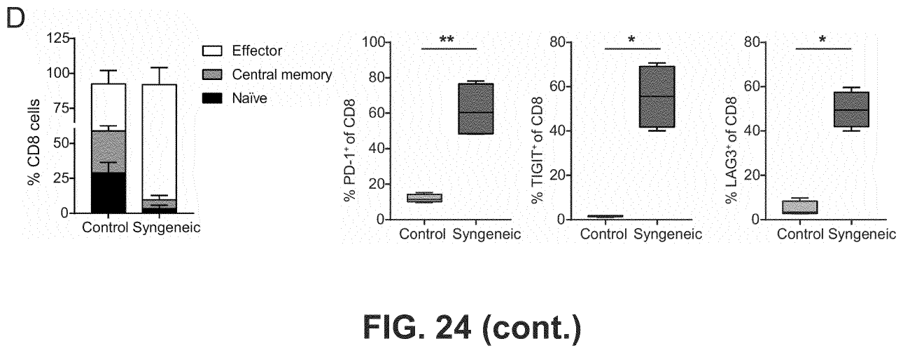

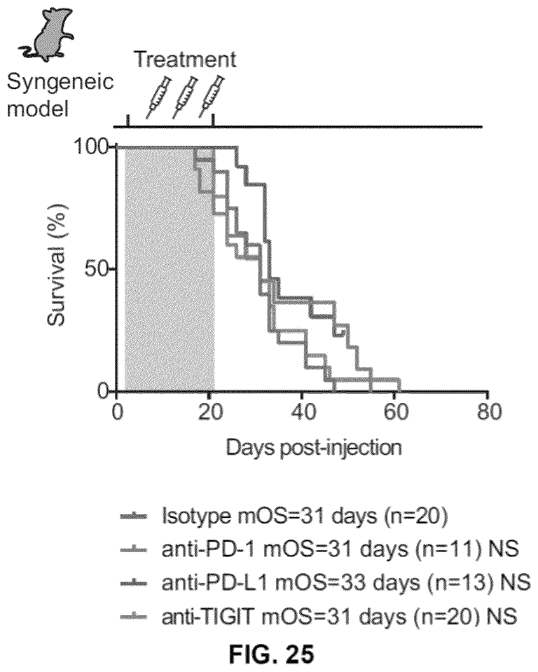

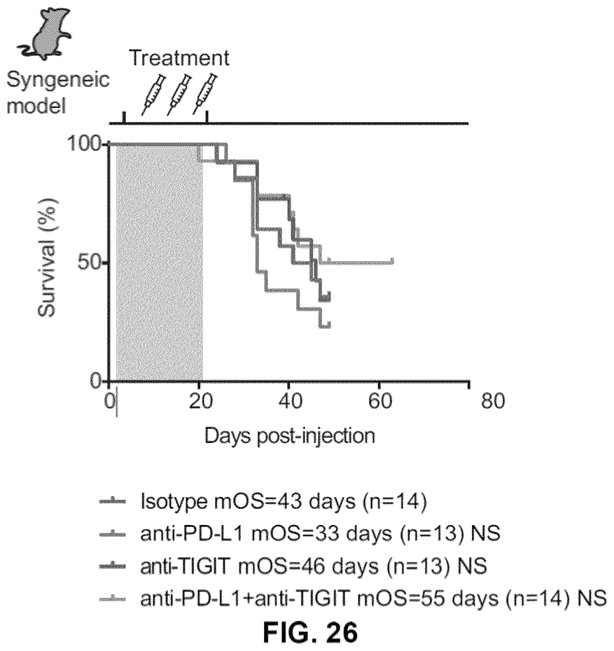

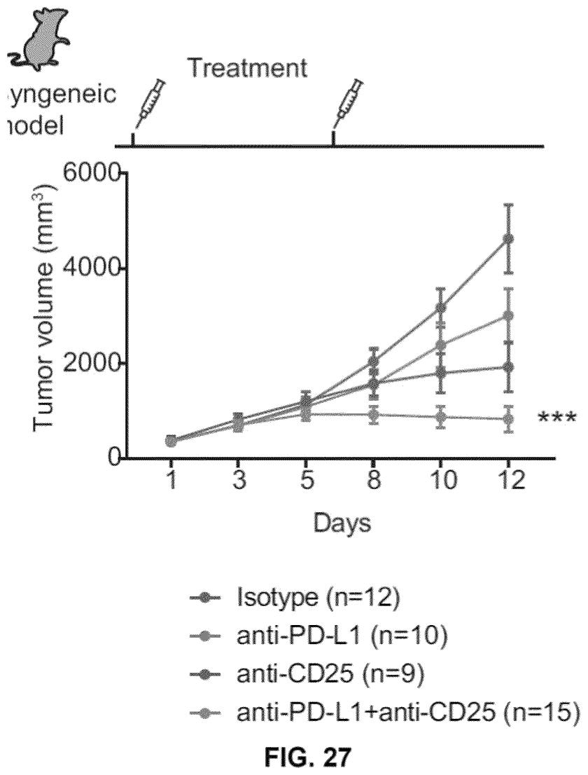

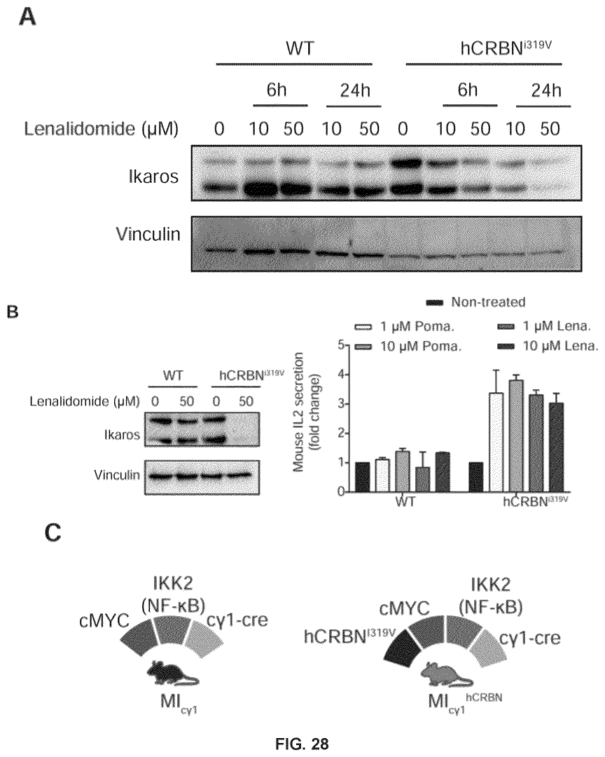

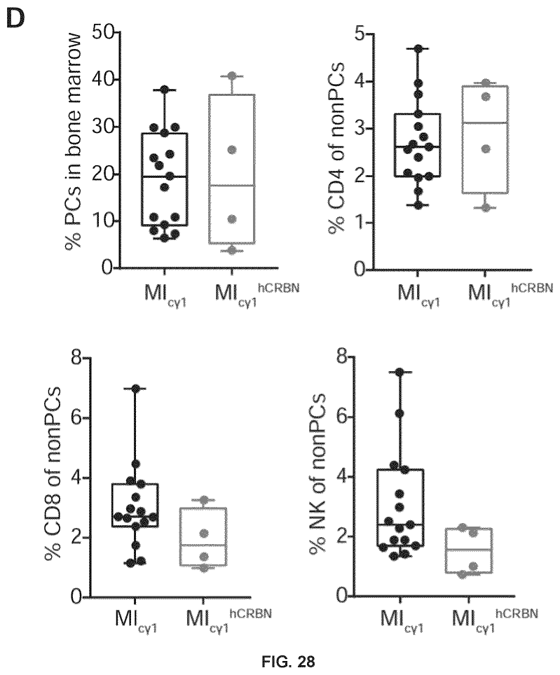

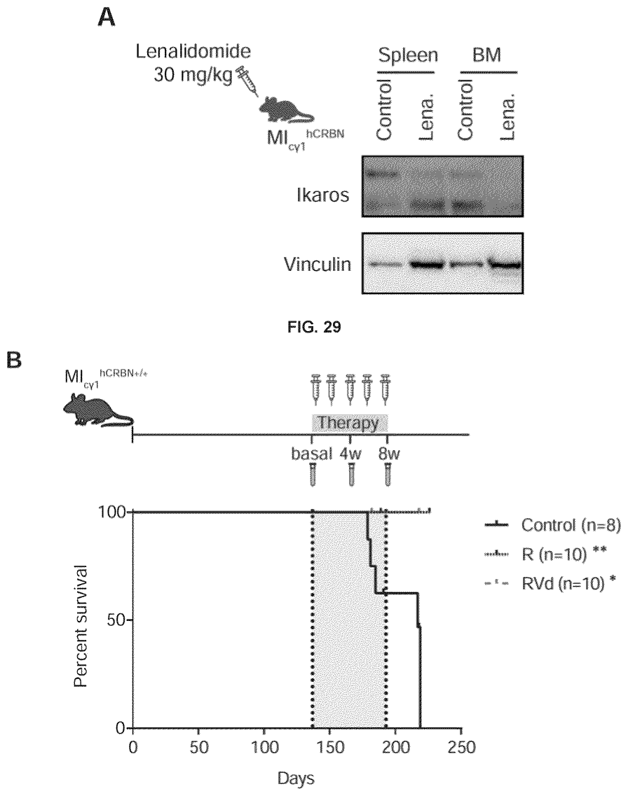

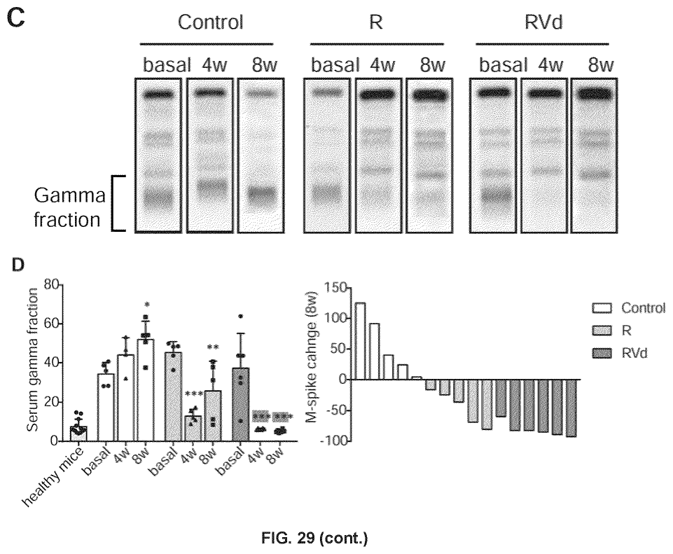

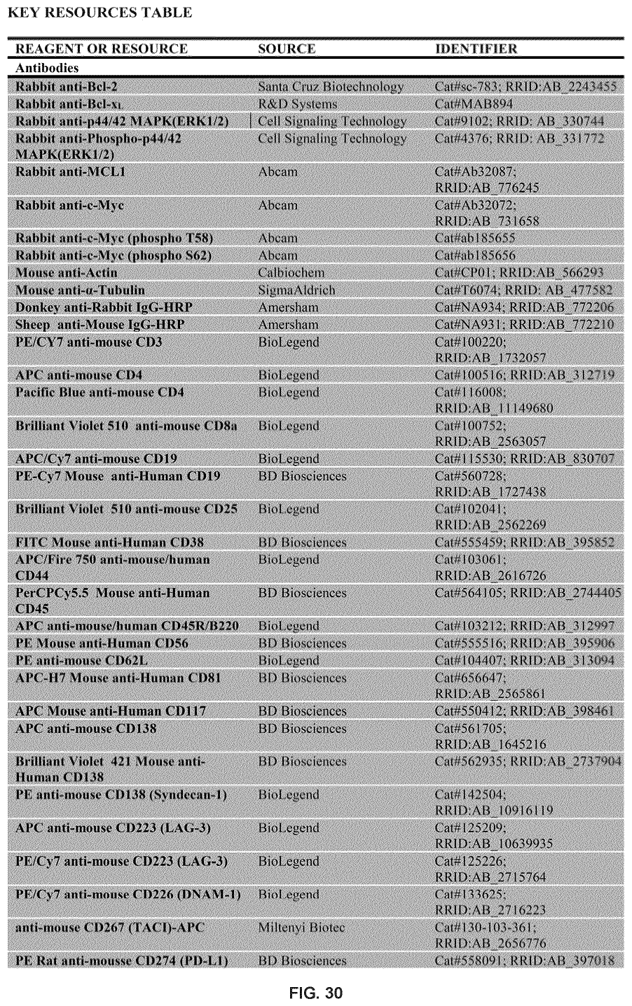

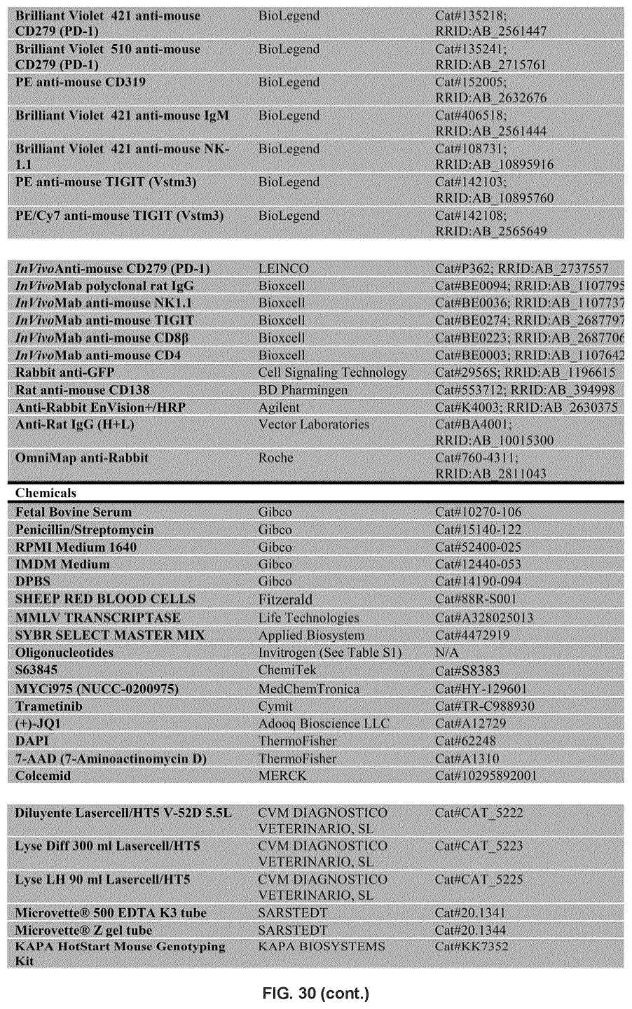

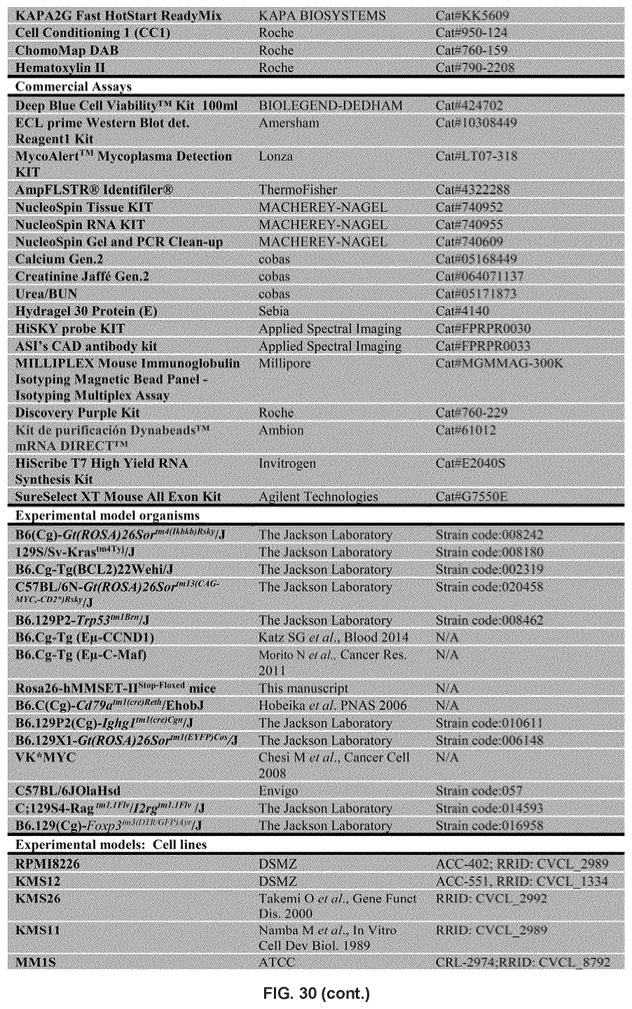

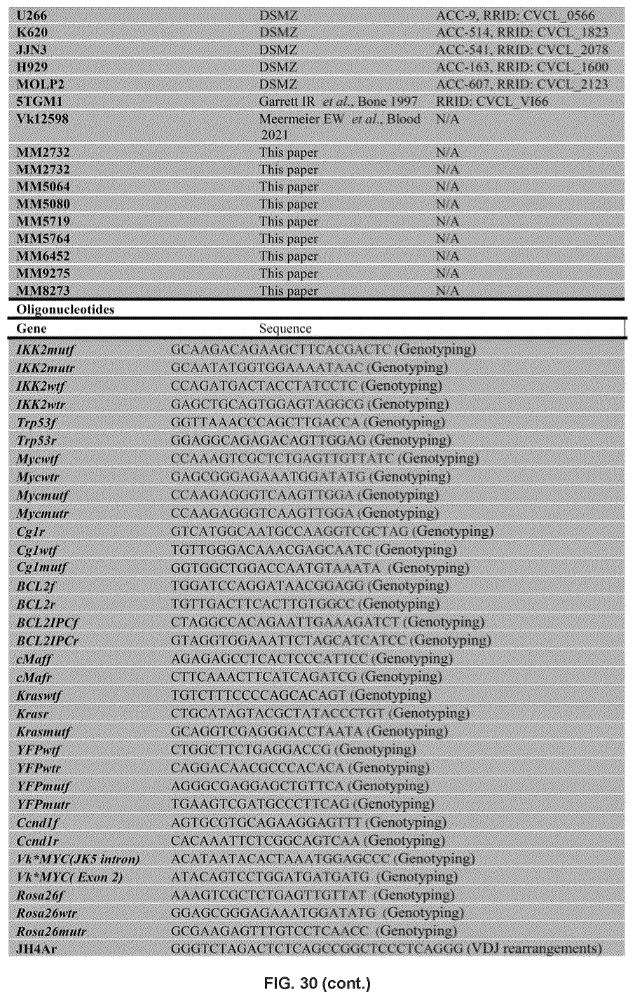

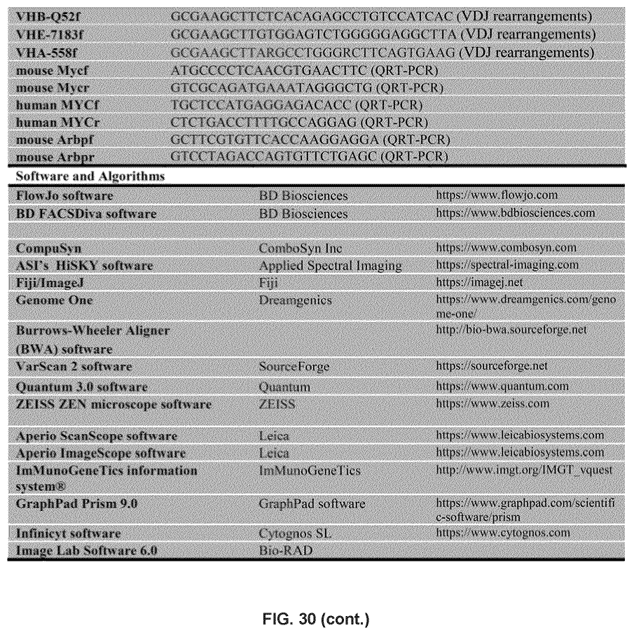

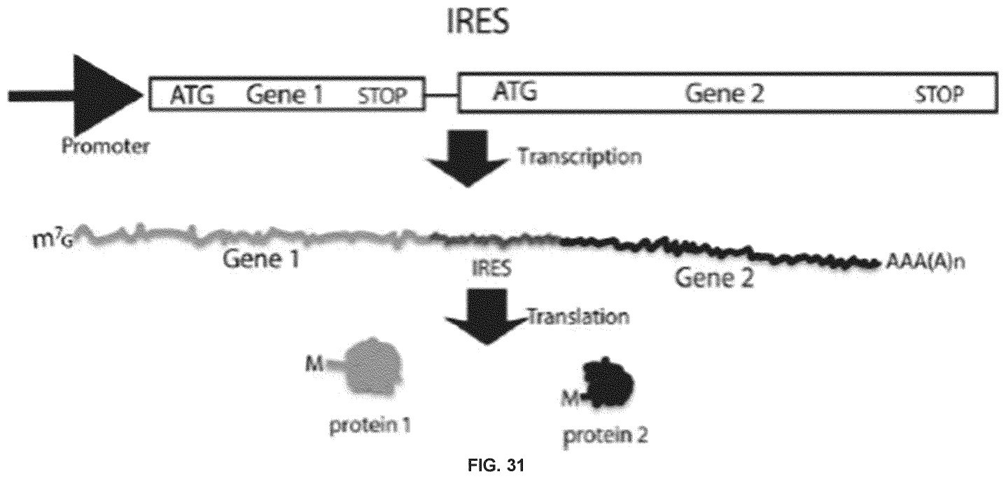

The results showed that:
Among the 14 mouse strains with two genetic lesions that were activated in combination at mature (n=12) or immature B (n=2) cells thorough cre-cγ1 or mb1/Cd79a-cre alleles, respectively, and were followed up for at least 1 year, only four developed tumors that fulfilled the criteria of classical MM. In addition, one strain developed atypical MM with IgM secretion (MM IgM), three developed B or T-cell leukemias/lymphomas, and the remaining 6 did not develop any tumor.
   Among the four models that developed classical MM, one model termed BIcγ1 was generated by the activation of a mouse IKK2 transgene by the cγ1 allele at germinal center B cells and the activation of a human BCL2 transgene by a Eμ promoter at immature B lymphocytes. The remaining models termed MIcγ1, MMSET-Icγ1 and MMSET-Mcγ1 were generated by the activation of the pair of the genetic lesions from mature germinal center B cells through the cγ1 recombinase.
   The four models that developed classical MM with secretion of IgG or IgA were driven by the cre-cγ1, while the atypical MM with IgM secretion was generated with the activation of IKK2 and MYC by the mb1/Cd79a-cre recombinase at immature B cells, which correspond to less than 1% of the cases observed in patients. Note that the activation of the same transgenes, IKK2 and MYC, from germinal center B cells by the cγ1 recombinase led to the development of classical MM with secretion of IgG or IgA.
Among the 14 models generated with the combination of 3 (n=13) or 4 (n=1) genetic lesions, triggered by a cγ1 (n=13) or mb1/Cd79a (n=1) cre-recombinase, 10 developed classical MM while the remaining 4 strains developed B or T-cell leukemias/lymphomas.
Nine strains developed lethal tumors classified as mature B-cell lymphoma or acute lymphoblastic leukemia (FIG. 3). Three of the remaining lines exhibited fully penetrant PC tumors in the BM, which shortened median overall survival (OS) to below 12 months of age (FIG. 1b).

Two of these mouse lines were termed MImb1 and MIcγ1, as they carry MYC and IKK2 expression by mb1-cre or cγ1-cre alleles, respectively, which indicates that NF-κB activation accelerated MYC-driven MM development compared to Vk*MYC mice (median OS, 197 and 208 days vs. 509 days; p<0.001). The third mouse line was termed BIcγ1, as this carries BCL2 and IKK2 expression by the cγ1-cre allele, and exhibited a median OS of 296 days, which indicates that apoptosis restriction in cells with NF-κB signaling was sufficient for transformation. BM tumors in the three different lines were composed of >10% GFP⁺CD138⁺B220⁻sIgM⁻ PCs, which morphologically resembled human MM cells; they also expressed typical MM markers including acid phosphatase, BCMA, SLAMF7, and TACI, secreted Igs into the serum, and exhibited clonal IgHV gene rearrangements (FIG. 1c-f and FIG. 4a-b). In addition, mice presented with common CRAB-like clinical features (hypercalcemia, Renal disease, Anemia, and Bone disease) (FIG. 4c). However, while the BIcγ1 and MIcγ1 strains predominantly secreted IgG or IgA, the MImb1 mice derived from immature pre-B cells presented with IgM-secreting MM (FIG. 1g and FIG. 4d). Genetic studies in patients with IgM MM, which corresponds to less than 1% of MM cases, showed a pre-germinal B lymphocyte origin, which is matched by the MImb1 model. In contrast, BIcγ1 and MIcγ1 mice developed class-switched MM from GC B lymphocytes that fulfill the diagnostic criteria of human disease, which implicates these cells in the origin of typical MM.

To build MM genetic heterogeneity, BIcγ1 and MIcγ1 strains were crossed with lines carrying additional MM genetic changes, including the common KrasG12D mutation and the high-risk P53 deletion. Both genetic abnormalities shortened the time to MM development in BIcγ1 and MIcγ1 mice, inducing a BM disease composed of GFP⁺CD138⁺B220⁻sIgM⁻ PCs that secreted IgG or IgA, and was classified as MM (FIG. 1h-i and FIG. 5a-b). Likewise, concomitant KrasG12D and P53 deletion in BIcγ1 mice rapidly induced BM and extramedullary PC tumors (FIG. 5a). These experimental results mimic data from patients with SMM, which indicates that MAPK-Ras mutations and P53 inactivation accelerate the onset of clinically active MM. Then we explored whether apoptosis restriction could influence MM development in MIcγ1 mice. To this end, transgenic BCL2 expression was added to the MIcγ1 strain, which yielded marked acceleration of MM onset (FIG. 1i and FIG. 5c). We further expanded the genetic heterogeneity by adding the three Ig chromosomal translocations used to stratify MM into genetic-risk groups. To achieve this, BIcγ1 mice were crossed with the Eu-CCND1, Eu-MAF or the newly generated Rosa26-hMMSET-IIStop-Floxed mouse lines, representative of standard-risk t(11;14) or the high-risk translocations t(14;16) and t(4;14), respectively. BIcγ1 mice carrying any of these three transgenes mouse lines carrying standard-risk t(11;14) or the two high-risk translocations t(14;16) and t(4;14). BIcγ1 mice carrying any of these three translocations developed BM tumors classified as typical MM, all of which exhibited overlapping survival curves (FIG. 1h and FIG. 6a). Similarly, a strain derived from MIcγ1 mice with additional t(14;16) developed MM and exhibited a similar survival to that of MIcγ1 mice (FIG. 1i and FIG. 6b). These experimental findings indicate that while Ig translocations contribute to MM development, they do not accelerate MM onset in mice. Likewise, SMM patients carrying t(11;14), t(14;16), or t(4;14) are not at increased risk of progression to active MM compared with those without Ig translocations. On the other hand, dysregulation of MMSET clearly contributed to MM initiation, as Rosa26-hMMSET-IIStop-Floxed mice crossed with lines carrying either IKK2NF-κB activation or MYC expression drove MM development (FIG. 1j and FIG. 7a). Taken together, we have developed a panel of fifteen mouse models fully encompassing MM genetic heterogeneity, including the standard- and high-risk genetic subgroups (FIG. 8).

### MM is Preceded by MGUS and SMM-Like Precursor States

We next determined whether, like in patients, precursor disease was present before the onset of symptomatic MM. In BIcγ1 and BIcγ1-derived mice, lethal MM was uniformly preceded by an MGUS-like stage from 6 months of age, characterized by minimal BM infiltration of oligoclonal GFP⁺CD138⁺B220⁻sIgM⁻ PCs that moderately secreted class-switched Igs into the serum (FIG. 1k-l and FIG. 7b). The number of PCs, the degree of IgHV clonality, and the levels of Igs increased over time and demarcated an SMM-like asymptomatic stage with >10% of clonal PCs, which eventually transformed into MM in 4 to 6 months. In contrast, MIcγ1 and MIcγ1-derived mice exhibited prominent MGUS-like disease in BM from 4-5 months of age that rapidly transformed into aggressive MM within several weeks (FIG. 1k-l and FIG. 7b,c). Thus, pre-malignant stages precede clinically evident MM in genetically heterogeneous mice. However, MIcγ1-derived models exhibited a rapid MGUS to MM transition, while the BIcγ1-derived strains were characterized by a longer time to progression, which in humans corresponds to the 10-30 years required by human MGUS cells undergoing MM transformation. In summary, our genetically diverse mice recapitulate the natural history and clinical evolution of human disease, including models of early and late MM progression from precursor states (FIG. 8).

### MYC Activation is a Unifying Feature Across MM Genetic Subgroups

RNA sequencing (RNA-seq) of MGUS and MM cells from MIcγ1 and BIcγ1-derived mice defined a common transcriptional signature with respect to normal BM PCs, including the upregulation of PC genes (i.e. Prdm1, Irf4, Xbp1, Sdc1 encoding CD138, Tnfrsf17 encoding BCMA, Tnfrsf13b encoding TACI, and Slamf7) and the downregulation of B-cell genes (i.e. Pax5 and CD19) (FIG. 9a). To compare murine tumors with human disease, RNA-seq was applied to malignant PCs from newly diagnosed MGUS and MM patients to define a human transcriptional signature with respect to normal BM PCs. Using principal component analysis (PCA), murine and human MGUS cells were mapped in between PCs and MM cells, which is indicative of a similar evolving transcriptional trajectory (FIG. 9b and FIG. 10a-b). Additionally, gene set enrichment analysis (GSEA) showed enrichment of transcriptionally deregulated mouse genes in the human MM expression signatures (FIG. 10c). These data indicate that mouse and human MM share a common transcriptional profile. We then characterized the transcriptional changes underlying the transition of MGUS into MM in the mouse models with different times to progression. BIcγ1-derived mice exhibited a linear transcriptional evolution as BM PCs progressed to MGUS cells and then to MM cells, concordant with the late progression. In contrast, MGUS and MM cells from MIcγ1 mice clustered closely and exhibited a reduced number of differentially expressed genes, concordant with the rapid progression (FIG. 9c). Comparative analyses of these two transcriptional patterns of progression revealed that the MYC oncogene was highly expressed in MM cells compared with MGUS cells in the BIcγ1-derived models, while transgenic MYC expression was already high in MGUS cells from MIcγ1 mice and remained stable during MM progression (FIG. 9d). GSEA of the MM transcriptomes found “MYC target genes” among the top hallmarks in both BIcγ1-derived and MIcγ1-derived models (FIG. 9e). Accordingly, MYC protein expression was detected in primary BM GFP⁺ MM cells and MM-derived cell lines established from primary MM samples, including early and late progressors (FIG. 9f, and FIG. 11). These results demonstrate the acquisition of endogenous MYC expression during MM progression in BIcγ1-derived models, while early activation of transgenic MYC in MIcγ1 mice accelerates MM progression. Likewise, in patients, MYC expression levels in MGUS cells were similar to those in BM PCs and were increased in MM cells (FIG. 9g), which is in agreement with previous studies, and confirms that MYC regulates time to progression into MM.

Genetic characterization of murine MM cells revealed hyperdiploid karyotypes with recurrent chromosomal gains and losses as well as complex structural rearrangements (FIG. 9h-i). These included human-like translocations between MYC and the IgH or IgL genes in 11 of 62(18%) primary MM samples and 3 of 6(50%) MM-derived cell lines (FIG. 9j). However, MYC chromosomal changes were not observed in MGUS cells, indicating that these were acquired during MM progression, as reported in patients (FIG. 12). We then evaluated the oncogenic function of MYC in genetically diverse MM-derived cell lines. Selective targeting of MYC with the small molecule MYCi975 induced dose-dependent MYC protein reduction, which decreased viability of murine and human MM cells (FIG. 9k). Therefore, MYC activation is a unifying feature in genetically heterogeneous MM, which distinguishes cases with early and late progression from precursor stages.

### A Common MAPK-MYC Genetic Axis is Amenable to Targeted Therapy

Quantification of the tumor mutation burden (TMB) by whole-exome sequencing (WES) revealed 28 somatic mutations per tumor in murine MGUS samples, 31 in MM samples, and 172 in MM243 derived cell lines. These included typical mutations in the FAM46C gene and in genes coding epigenetic modulators and cadherins (FIG. 13a). Mutations in genes in the NF-κB pathway were observed in one of 31 MM samples, which indicates that moderate activation of NF-κB signaling by a heterozygous IKK2 allele is enough for the development of precursor stages, which progress into MM without additional changes in the pathway. In clear contrast, mutations in genes in the MAPK pathway were observed in 29 of 62(47%) mice at the MM stage; these rates are similar to those observed in MM patients 4, which suggests that mutations in this signaling cascade accumulate during MM progression. Analysis of the genomic characteristics among MM from the models of early and late progression revealed that MIcγ1 mice exhibited normal karyotypes without MYC translocations, while BIcγ1 mice with P53 deletion exhibited higher numbers of chromosomal abnormalities and TMB compared with the strains without P53 deletion (FIG. 13b). Concordantly, among 599 MM patients in the CoMMpass study (NCT01454297), those carrying del (17p) and/or P53 mutations exhibited higher copy number changes and TMB compared with the remaining patients (FIG. 13b), indicating that P53-driven genetic instability promotes MYC rearrangements during MM progression.

We next asked whether the acquired MAPK mutations were analogous in mouse and human MM. Of the 34 MAPK genes with mutations in murine MM, 19 (56%) were recurrently mutated in patients in the CoMMpass study. Accordingly, Western blot analyses identified consistent phosphorylation of the protein kinase ERK, a surrogate of MAPK activation, in murine and human MM-derived cell lines (FIG. 13c). Moreover, targeting MAPK signaling with trametinib, a MEK-ERK inhibitor approved for BRAF-mutated melanoma, reversed ERK phosphorylation and reduced mouse and human MM cell growth, which indicates shared MAPK activation (FIG. 13d-e).

Given that mutations in MAPK pathway and MYC activation are acquired during MM development, we investigated whether MAPK signaling could modulate MYC expression. Although trametinib did not consistently change MYC gene expression at the RNA level, MEK inhibition decreased phosphorylation of MYC at Ser62, which induced dose-dependent MYC degradation (FIG. 13f). These results indicate that while P53 inactivation triggers transcriptional MYC activation through chromosomal changes, constitutive MAPK signaling stabilizes MYC protein during MM development. These data are in accordance to the P53/Kras-BIcγ1 mouse model (see FIG. 1h), which showed that simultaneous P53 loss and K-rasG12D cooperated to accelerate MM onset.

### Immunological Features of the Bone Marrow Microenvironment in Multiple Myeloma

Immune surveillance restricts clinical progression in MGUS and SMM patients for extended periods. To give further insights from the models, sequential changes in the BM immune microenvironment were determined by multi-parametric flow cytometry in mice with different genotypes at different disease stages. A linear increase in the number of T lymphocytes and NK cells were observed during progression, which correlated with PC expansion (FIG. 14a-b). CD8⁺ T cells acquired a CD44⁺CD62L⁻ effector phenotype and sequentially expressed the exhaustion markers PD-1, TIGIT and LAG3, while NK cells also exhibited activated phenotypes (FIG. 15a-c). Due to the continuous range of T and NK cell infiltration observed in the BM microenvironment in different mouse strains, we used a classification of immune phenotypes in solid tumors to assign MM cases to immune-cold and inflamed subgroups (FIG. 14c). Immune-cold cases (25 of 59, 42%) exhibited a lymphoid cell infiltrate that resembled the BM immune microenvironment of healthy mice, while the immune-inflamed subgroup (34 of 59, 58%) was characterized by more abundant lymphoid cells, primarily tumor-reactive CD8⁺ T lymphocytes with exhausted phenotypes (FIG. 14d and FIG. 15d). In addition, immune-inflamed cases contained higher number of immunosuppressive CD4⁺CD25⁺Foxp3⁺ regulatory T (T_{reg}) cells. The burden of activated CD8⁺ T lymphocytes, but not of NK cells, correlated with the number of T_{reg} cells, which suggests that T-cell cytotoxic and immunosuppressive states interact during murine MM development (FIG. 14d and FIG. 15e).

To explore similarities with human disease, we examined the BM immune microenvironment in primary samples from patients newly diagnosed with MGUS (n=108), SMM (n=167), or MM (n=652) by multi-parametric flow cytometry. A progressive increase in T and NK cell populations was observed during the progressive MM stages, which correlated with MM cell burden (FIGS. 14e-f). According to the cancer immune-phenotype classification described above, the cohort of MM patients was divided into those with immune-cold and those with inflamed tumors (FIG. 14g). Of 652 MM cases, 435 (67%) were classified as immune-cold, as they were characterized by T and NK cell infiltrates that matched those in healthy donors. In contrast, the remaining cases (33%) corresponded to immune-inflamed MM, which was defined by a higher number of tumor-reactive CD4⁺ and CD8⁺ T lymphocytes and NK cells (FIGS. 14g-h and FIG. 15f). Mimicking results in mice, the number of T_{reg} cells was higher in the immune-inflamed category and was correlated with the abundance of CD8⁺ T lymphocytes, but not with NK cells (FIG. 14h and FIG. 15g). The presence of the immune-cold and inflamed MM subgroups was validated in a previously reported clinical series of MM patients (FIG. 16). In summary, remodeling of the BM immune microenvironment during progression classifies murine and human disease into distinct immune categories.

Next, we investigated whether these categories were associated with MM biological and clinical characteristics. Immune-inflamed cases exhibited higher levels of monoclonal Ig in serum, as a surrogate of the increased MM cell burden (FIG. 14i). In addition, the BM immune phenotypes correlated with age, being the quantity of the BM infiltrating T and NK lymphocytes negatively correlated with increasing age (FIG. 14j and FIG. 17a). However, in mouse models and patients, the distribution of the immune-cold and inflamed phenotypes and the tumor-reactive lymphoid cell infiltrates were similar among the MM genetic subgroups, including the standard- and high-risk categories (FIG. 14k). Additionally, and contrary to other cancers, quantification of the TMB from WES analyses in MM cells did not reveal a correlation with BM immune features (FIG. 17b-d). In conclusion, MM immune categories correlate with the number of tumor cells and with aging, but not with the genetic-risk groups or the TMB.

### A CD8+ T Cell Versus Treg Cell Ratio Modulates Immunotherapy Responses

We then asked whether early and late MM progression in the MIcγ1 and BIcγ1 models could influence responses to immunotherapy, and particularly to immune checkpoint blockade (ICB) therapy. To this end, pre-clinical in vivo immunotherapy trials using monoclonal antibodies (moAbs) to inhibit the two immune checkpoint receptors PD-1 and TIGIT were performed. In MIcγ1 mice, anti-PD1 therapy started at MGUS stage and continued during 8 weeks significantly reduced tumor burden and delayed MM development in treated vs. untreated animals (mOS, 258 vs. 197 days; p<0.05) (FIG. 18a). In contrast, anti-PD-1 therapy in the BIcγ1 strain started at the MGUS stage did not induce responses in the treated cohort compared with control mice (mOS, 286 vs. 302 days; p=0.61) (FIG. 18b). Similar therapy strategies with the anti-TIGIT moAb did not yield therapeutic benefits in MIcγ1 or BIcγ1 mice (FIG. 18a-b). We investigated whether the composition of the BM microenvironment at precursor stages modulated ICB responses. MIcγ1 mice exhibited higher numbers of activated PD-1⁺TIGIT⁺LAG3⁺ CD8⁺ T lymphocytes compared with BIcγ1 mice, concordat with an immune-inflamed phenotype, but also showed a lower number of immunosuppressive CD4⁺PD-1⁺ T_{reg} cells (FIG. 18c and FIG. 19). Accordingly, the ratio of CD8⁺ T cells to T_{reg} cells was markedly higher in MIcγ1 than in BIcγ1 mice (median value of CD8/T_{reg} cell ratio, 22.5 vs. 6.1; p=0.019) (FIG. 18d). In this setting, in vivo depletion of CD8⁺ T lymphocytes, but not of CD4⁺ T cells, accelerated MM onset in MIcγ1 mice (FIG. 20a). In contrast, depletion of CD8⁺ T cells did not modify survival of BIcγ1 mice, but rather, survival was extended upon CD4⁺ T-cell depletion (FIG. 20b). These findings show that the abundance of tumor-reactive CD8⁺ T cells vs. the immunosuppressive T_{reg} cells characterized the rapid model of MM progression driven by MYC activation, which favored the activity of anti-PD-1 therapy. Because MYC can regulate the immune response by repressing PD-L1 transcription in tumor cells, we investigated this possibility in the mouse models. Pharmacological inhibition of MYC repressed PD-L1 expression at transcriptional and protein levels in MM cells from MIcγ1 mice (FIG. 18e). These results conclude that early MYC activation triggered PD-L1 expression in MM cells to evade cytotoxic CD8⁺ T-cell surveillance via PD-1 blockade, thereby explaining the selective efficacy of PD-1 inhibition.

To explore this concept in patients, flow cytometry analysis was carried out in the BM of 69 smoldering MM patients who were followed up without receiving treatment. Patients with a high CD8/T_{reg} cell ratio exhibited a shorter time to progression into active MM with respect to the cases with low ratios (median progression free survival—PFS—at 2 years, 38% vs. 88%; p=0.005) (FIG. 18f). These results indicate that a rapid progression in SMM occurs through the blockade of PD1⁺CD8⁺ T lymphocytes by the tumor cells irrespectively of T_{reg} cells, and suggest that SMM patients with high-risk of progression may benefit from anti-PD1 therapy. Then, the ratio of BM CD8⁺ T cells vs. T_{reg} cells was investigated in patients with clinically active MM. Among 170 cases, 23(14%) exhibited a higher T-cell ratio like in MIcγ1 mice, while the remaining patients (143 cases, 86%) showed lower ratios comparable to those in BIcγ1-derived mice (FIG. 18g). The presence of a high CD8⁺/T_{reg}-cell ratio predicting ICB responsiveness in only 14% of patients may provide a scientific rationale to the negative results of the anti-PD-1 moAb in past clinical trials. We then examined whether the BM T-cell ratio could influence clinical responses to standard-of-care therapy. Among 170 MM patients treated with lenalidomide and dexamethasone, those with a high BM CD8/T_{reg} cell ratio showed better responses compared with those with low values (PFS, not reached vs. 26 months; p=0.225) (FIG. 18h). In an additional clinical series of MM patients, those patients with a BM immune microenvironment overlapping with that of MIcγ1 mice exhibited superior outcome under lenalidomide/thalidomide-containing regimens compared with the remaining cases (median PFS, 85 months vs. 62 months; p=0.0005) (FIG. 18i and FIG. 21). These findings demonstrate that the time to progression from precursor stages into MM shapes the BM immune microenvironment, which in turn influences clinical immunotherapy outcomes.

### Targeting the Multiple Myeloma Immune Microenvironment

To directly compare the BM immune portraits in mouse and human MM, we performed bioinformatic deconvolution of bulk RNA-seq data to reconstruct the tumor microenvironment (TME) in samples from newly diagnosed MM patients and from mice of different genotypes developing MM. Integrative studies classified the TME of MM patients and mice into distinct overlapping immune subgroups, allowing all the 28 mouse samples to be matched to 307 (87%) of the 354 human MM samples.

To explore the functional interaction between T cell subsets in the TME, single-cell RNA-seq coupled with T cell antigen receptor (TCR) sequencing (scRNA-seq/TCR-seq) was conducted in BM CD3+ T lymphocytes from mice (n=60,858 cells) and patients (n=50, 154 cells) at the MGUS and MM stages, along with BM T cells from mouse and human healthy controls (FIGS. 18j, 22 and 23a). In MIcγ1 and BIcγ1 mice, markers of exhaustion/activation (Pdcd1, Tigit, Lag3) and cytotoxicity (Ifng, Gzma, Gzmb, Gzmk) were similarly expressed by CD8+ T cells at MM states, but these were barely detected in MGUS samples. Treg cells from both mouse models also expressed markers of an activated/immunosuppressive state, including Tigit, Ctl4, Cxcr3, Tnfrsf9 (encoding Cd137), Icos and Tnfrsf4 (encoding OX40). Intriguingly, such a Treg cell-activated phenotype was already evident in MGUS samples and maintained in the MM stage in both MIcγ1 and BIcγ1 mice (FIG. 23b). In patients, such early activation of T_{reg} cells was also evidenced at MGUS and MM states, in contrast to the phenotype of CD8+ T lymphocytes, which was minimally activated/exhausted at the MGUS state and became fully exhausted at the MM state). In this setting, frequent clonotypic TCR sequences were found among CD8+ T cells in mice and patients, which were already present at the MGUS stage, suggesting a tumor antigen-driven function. In contrast, the number of clonal TCR sequences was markedly lower in Treg cells. Functional ex vivo assays in mouse cells demonstrated the immunosuppressive capacity of Treg cells over CD8+ T lymphocytes, while the latter exhibited MM cell-specific immune recognition. Further, by applying major histocompatibility complex (MHC)-binding predictive algorithms to nonsynonymous single-nucleotide variations (SNVs) identified by exome sequencing data from two mouse MM cell lines, we identified potential neoantigens with high binding capacity to MHC class I and/or class II molecules, a fraction of which were functionally validated as having specific T cell immunogenicity

We next explored whether disturbing the balance between T-cell cytotoxicity and immunosuppression would affect the response to ICB. To this end, anti-PD-1-resistant syngeneic transplants were established by intravenous injection of BIcγ1-derived MM cell lines into immunocompetent mouse recipients. Mice from one of the syngeneic models accumulated MM cells in the BM, along with abundant PD-1⁺TIGIT⁺LAG3⁺CD8⁺ T cells and a high number of PD-1⁺T_{reg} cells (FIG. 24). In this context, no response to moAbs inhibiting PD-1, PD-L1 and TIGIT was observed, in accord to the distribution of T-cell subsets in the BM microenvironment (FIG. 25). Depletion of CD8⁺ T cells markedly accelerated MM onset, while genetic depletion of T_{reg} cells in vivo in Foxp3-GFP-DTR mice delayed MM development, suggesting a role of CD8⁺ T-cells and T_{reg} cells in the control of MM cells (FIG. 18k). Accordingly, mitigating CD8⁺ T-cell exhaustion via TIGIT co-inhibition led to responses to both PD-1 and PD-L1 blockade, achieving durable MM responses (FIG. 18I and FIG. 26). Moreover, depletion of T_{reg} cells with an anti-CD25 moAb extended survival of mice, and enhanced efficacy of anti-PD-1 and anti-PD-L1 treatments, leading to long term responses (FIG. 18m and FIG. 27). Collectively, these data reinforce the notion that the BM CD8/T_{reg} cell ratio predicts ICB responsiveness, and provides a potential biomarker to optimize MM immunotherapy in the clinic.

### Humanized Multiple Myeloma Mouse Models

An additional step being carried out is the generation of new models of MM carrying humanized genes that encode for proteins that are targeted by different drugs or compounds in pre-clinical or clinical development. These include the following target proteins: CRBN, CD38, BCMA, SLAMF7, GPRC5D and FcRH5. To generate humanized models of MM with the corresponding humanized protein, we have manipulated the genome of mouse cells to substitute the mouse gene sequence by the human gene sequence, with the expectation that the mouse cells will now express the human protein. Depending on the case, the full open reading frame of the gene, or only the part encoding for a selected domain has been substituted. As an example (shown in FIG. 28), the mouse CRBN gene (which encodes for the protein CRBN, which is the target of immunomodulatory drugs including lenalidomide, pomalidomide and thalidomide) was replaced by the human CRBN gene. In this case, only a small fraction of the mouse gene encoding for one amino acid was replaced by the human gene sequence, leading to the generation of the I391V variant, whereby the original mouse isoleucine (I) will be replaced by a valine (V) at codon 391 (the generation of these mice is described in Fink et al. Blood 2018 Oct. 4; 132(14):1535-1544; PMID: 30064974). Accordingly, mouse cells from the hCRBNI391V mice are response to lenalidomide, as shown by the dose-dependent degradation of Ikaros protein (a typical CRBN target) upon treatment, which is not observed in wild-type cells. Similar effects are also observed in T cells from hCRBNI391V mice, which show increase of IL2 secretion upon lenalidomide exposure, which is not observed in wild-type cells.

When crossing hCRBNI391V with MIcγ1 mice, the result in the new MIcγ1 hCRBN mouse strain, which develops MM and shows responses to treatment with lenalidomide in vitro (FIG. 29a). In addition, MIcγ1 hCRBN mice exhibit significant prolonged overall survival of mice with respect to untreated MIcγ1 hCRBN mice or to MIcγ1 mice without hCRBN treated with lenalidomide in vivo (FIG. 29b-d). Following this example, we have now designed and are already generating other new mice for substituting mouse CD38, BCMA, SLAMF7, GPRC5D and FcRH5 gene sequences by the human corresponding gene sequences.

In addition, we have designed new humanized models of drug targets in the immune cells of the tumor microenvironment, particularly in T cells, including CD3, CD28, 4-1-BB1, PD-1 and PD-L1. These molecules are currently being used and/or are being evaluated as immunotherapy targets for the treatment of different cancers including MM, particularly with mono and bi-specific antibodies and CAR T cell therapies. Following a similar strategy as reported above for humanized CRBN, gene sequences of the target genes in the mouse genome will be replaced by the human gene sequence, either the full open reading frame or a portion of the sequence, generally including the extracellular part of the protein. In the case of CD3, as an example, the mouse gene fragment encoding for the CD38 chain will be replaced by the human gene fragment encoding for the same protein segment. The huCD3ε mice will be then crossed to MIcγ1 mice to generate the new MIcγ1 huCD3ε mouse strain, which will develop MM and will allow testing monoclonal antibodies in clinical use against human CD3.

All the resources and reagents used in this study are listed in the Key Resources Table shown in FIG. 30.

Bi-cistronic vectors: To accelerate the process of generating genetically diverse mice, we have designed bi-cistronic vectors that are composed of two different transgenes cloned together, which are regulated by a common promoter (FIG. 31). Four bi-cistronic vectors have been designed, which are framed in FIG. 8 in red, and correspond to BI, MI, Mmset-I and Mmset-M mice. To this end we have started the genetic manipulation of mouse cells to insert the bi-cistronic vectors with the two cloned genes into the permissive Rosa26 mouse locus at chromosome 6. Once generated, mice will be crossed with cγ1-cre or mb1-cre mice, which will activate the double transgenes in the corresponding target cells, thereby inducing the development of MM. It is expected that the disease in these mice will be similar to that observed in the original mice resulting from conventional breeding.

As an example, BCL2 and IKK2 transgenes will be cloned together in the bi-cistronic vector carrying a common promoter, and inserted into the Rosa26 loci to generate BI_{cis} mice. Then, BI_{cis} mice will be crossed with cγ1-cre mice to generate BI_{cis}-cγ1 mice, which will induce MM development as in the original BIcγ1 mice, but in a shorter period of time as a consequence of reducing the breeding that will also diminish the costs. Likewise, MIcis, Mmset-I_{cis} and Mmset-M_{cis} mice will be generated by inserting the corresponding bicistronic vector into the Rosa26 locus.

1.-92. (canceled)

93. A genetically engineered mouse comprising:
(i) a conditionally activatable transgene encoding a constitutively active form of the IKK2 protein and a transgene encoding the human BCL2 protein,
(ii) a conditionally activatable transgene encoding a constitutively active form of the IKK2 protein and a conditionally activatable transgene encoding the MYC protein,
(iii) a conditionally activatable transgene encoding a constitutively active form of the IKK2 protein and a conditionally activatable transgene encoding the MMSET-II protein,
(iv) a conditionally activatable transgene comprising the MYC protein coding sequence and a conditionally activatable transgene encoding the MMSET-II protein, or
(v) a conditionally activatable transgene encoding the human MMSET-II protein.

94. The genetically engineered mouse according to claim 93 wherein when in relation to the genetically engineered mouse (i) it further comprises at least one of the following:
a conditionally activatable transgene encoding the K-Ras^{G12D} protein,
a conditionally inactivatable Trp53 gene,
a transgene encoding the Cyclin-D1 protein,
a transgene encoding the MAF protein, or
a conditionally activatable transgene encoding the MYC protein,
or wherein when in relation to the genetically engineered mouse (ii) further comprises at least one of the following:
a conditionally activatable transgene encoding the K-Ras^{G12D} protein,
a conditionally inactivatable Trp53 gene,
a transgene encoding the BCL2 protein, or
a transgene encoding the MAF protein.

95. The genetically engineered mouse according to claim 93 wherein the one or more transgenes that are conditionally activatable comprise a region comprising a transcription terminator site flanked by recombinase target sites, said site being located at a position which causes premature termination of the transcription.

96. The genetically engineered mouse according to claim 95 wherein the transcription terminator site is a polyA sequence.

97. The genetically engineered mouse according to claim 93 additionally comprising within its genome a sequence encoding for a recombinase which is specific for the recombinase target sites flanking the transcription terminator sequence within the conditionally activatable transgene or transgenes.

98. The genetically engineered mouse according to claim 97 wherein the recombinase is the Cre recombinase and the recombinase target sites are loxP sites.

99. The genetically engineered mouse according to claim 93 wherein the sequence encoding the recombinase is placed under the operative control of a promoter specific for immature pre-B cells or for mature germinal center (GC) B lymphocytes; preferably for mature germinal center (GC) B lymphocytes.

100. The genetically engineered mouse according to claim 93(i) wherein the conditionally activatable transgene encoding a constitutively active form of the IKK2 protein comprises a region comprising a transcription terminator site, said region being located at a position which causes premature termination of the transcription, wherein said transcription terminator site is flanked by recombinase target sites, and wherein the genetically engineered mouse additionally comprises within its genome a sequence encoding for a recombinase which is specific for the recombinase target sites flanking the transcription terminator sequence within the conditionally activatable transgene encoding a constitutively active form of the IKK2 protein and wherein the sequence encoding the recombinase is placed under the operative control of a promoter specific for mature germinal center (GC) B lymphocytes.

101. The genetically engineered mouse according to claim 99 wherein the placing of the recombinase coding sequence under the control of a promoter specific of B lymphocytes is achieved by inserting the recombinase coding sequence into the cγ1 locus or into the mb1 locus, or wherein the recombinase coding sequence is under the control of the Cgamma1 protein (Cγ1-cre) promoter or of the Cd79a protein (mb1-cre) promoter.

102. The genetically engineered mouse according to claim 93 wherein the transgene encoding the BCL2 protein is under the control of the Eμ immunoglobulin heavy chain enhancer and the SV40 promoter.

103. The genetically engineered mouse according to claim 93 wherein the constitutively active form of the IKK2 protein contains the S177E and S181E mutations.

104. The genetically engineered mouse according to claim 93 wherein the transgene encoding a constitutively active form of the IKK2 protein is under the operative control of the Rosa26 promoter.

105. The genetically engineered mouse according to claim 93 wherein the conditionally activatable transgene encoding the MYC protein is inserted in the Rosa26 locus.

106. The genetically engineered mouse according to claim 93 wherein the MYC protein is the human MYC protein.

107. The genetically engineered mouse according to claim 93 wherein the conditionally activatable transgene encoding the MYC protein is under the control of the CAG promoter.

108. The genetically engineered mouse according to claim 94 wherein the conditionally activatable transgene encoding the K-Ras^{G12D} protein is located in the endogenous Kras locus replacing the wild-type Kras gene and under the operative control of the Kras promoter.

109. The genetically engineered mouse according to claim 94 wherein the conditionally inactivatable Trp53 gene is replacing the endogenous Trp53 gene, wherein the conditionally inactivatable Trp53 gene comprises recombinase target sites flanking a region in the gene which is required for the expression of a functional Trp53 protein and wherein conditionally inactivatable Trp53 gene is found under the operative control of the Trp53 promoter.

110. The genetically engineered mouse according to claim 109 wherein the region which is flanked by recombinase target sites comprises exons 2 to 10 of the Trp53 gene.

111. The genetically engineered mouse according to claim 93 wherein one or more of the conditionally activatable transgenes further comprise a reporter gene that is co-expressed upon activation of the transgene.

112. The genetically engineered mouse according to claim 94, wherein the transgene encoding the MAF protein is under the operative control of the Eμ immunoglobulin heavy chain enhancer and the VH promoter and/or wherein the transgene encoding the Cyclin-D1 protein is under the operative control of the Eμ immunoglobulin heavy chain enhancer and the VH promoter.

113. The genetically engineered mouse according to claim 93 wherein the conditionally activatable transgene encoding the MMSET-II protein is under the operative control of the CAG promoter.

114. The genetically engineered mouse according to claim 94, additionally comprising within its genome a sequence encoding a recombinase which is specific for the recombinase target sites flanking the transcription terminator sequence within the conditionally activatable transgene or transgenes and/or specific for the recombinase target sites present in the Trp53 gene region which is required for the expression of a functional Trp53 protein.

115. The genetically engineered mouse according to claim 93 selected from the group consisting of:
(i) a genetically engineered mouse that comprises a conditionally activatable transgene encoding a constitutively active form of the IKK2 protein and a transgene encoding the human BCL2 protein,
(ii) a genetically engineered mouse that comprises a conditionally activatable transgene encoding a constitutively active form of the IKK2 protein, a transgene encoding the human BCL2 protein and a conditionally activatable transgene encoding the K-RasG12D protein,
(iii) a genetically engineered mouse that comprises a conditionally activatable transgene encoding a constitutively active form of the IKK2 protein, a transgene encoding the human BCL2 protein and a transgene comprising a conditionally inactivatable sequence encoding the p53 polypeptide,
(iv) a genetically engineered mouse that comprises a conditionally activatable transgene encoding a constitutively active form of the IKK2 protein, a transgene encoding the human BCL2 protein, a transgene comprising a conditionally inactivatable sequence encoding the p53 polypeptide and a conditionally activatable transgene encoding the K-RasG12D protein,
(v) a genetically engineered mouse that comprises a conditionally activatable transgene encoding a constitutively active form of the IKK2 protein, a transgene encoding the human BCL2 protein and a transgene encoding the Cyclin-D1 protein,
(vi) a genetically engineered mouse that comprises conditionally activatable transgene encoding a constitutively active form of the IKK2 protein, a transgene encoding the human BCL2 protein and a transgene encoding the MAF protein.
(vii) a genetically engineered mouse that comprises a conditionally activatable transgene encoding a constitutively active form of the IKK2, a transgene encoding the human BCL2 protein and a conditionally activatable transgene encoding the MMSET-II protein
(viii) a genetically engineered mouse that comprises a conditionally activatable transgene encoding a constitutively active form of the IKK2, a transgene encoding the human BCL2 protein and a transgene encoding the MYC protein,
(ix) a genetically engineered mouse that comprises a conditionally activatable transgene encoding a constitutively active form of the IKK2 protein and a transgene encoding the MYC protein,
(x) a genetically engineered mouse that comprises conditionally activatable transgene encoding a constitutively active form of the IKK2 protein, a transgene encoding the MYC protein and a conditionally activatable transgene encoding the K-RasG12D protein,
(xi) a genetically engineered mouse that comprises conditionally activatable transgene encoding a constitutively active form of the IKK2 protein, a transgene encoding the MYC protein and a transgene comprising a conditionally inactivatable sequence encoding the p53 polypeptide,
(xii) a genetically engineered mouse that comprises conditionally activatable transgene encoding a constitutively active form of the IKK2 protein and a conditionally activatable transgene encoding the MMSET-II protein, and
(xiii) a genetically engineered mouse that comprises a transgene encoding the MYC protein and conditionally activatable transgene encoding the MMSET-II protein.

116. The genetically engineered mouse according to claim 115 wherein:
the mouse as defined in any of (i) to (viii) or (x) to (xiii) contains the recombinase coding sequence under the control of the Cgamma1 protein (Cγ1-cre) promoter or
the mouse according to (ix) contains the recombinase coding sequence under the control of the Cd79a gene promoter,
the mouse as defined in any of (i) to (viii) contains the transgene encoding the BCL2 protein under the control of the Eμ immunoglobulin heavy chain enhancer and the SV40 promoter.

117. The genetically engineered mouse according to claim 112 wherein:
the mouse as defined in any of (i) to (viii) or (x) to (xiii) contains the recombinase coding sequence under the control of the Cgamma1 protein (Cγ1-cre) promoter or
the mouse according to (ix) contains the recombinase coding sequence under the control of the Cd79a gene promoter,
the mouse as defined in any of (i) to (viii) contains the transgene encoding the BCL2 protein under the control of the Eμ immunoglobulin heavy chain enhancer and the SV40 promoter.

118. The genetically engineered mouse according to claim 93 further comprising a transgene encoding a human or humanized protein which confers sensitivity to an anti-cell blood cancer therapy, said transgene being inserted in the locus of the non-human mouse orthologue.

119. The genetically engineered mouse according to claim 118 wherein the protein which confers sensitivity to an anti-cell blood cancer therapy is Crbn^{I391V}.

120. The genetically engineered mouse according to claim 118 wherein the protein which confers sensitivity to an anti-cell blood cancer therapy is a protein which is expressed in the surface of malignant plasma cells or in the surface of cells of the immune system.

121. The genetically engineered mouse according to claim 120 wherein the protein which is expressed in the surface of malignant plasma cells is BCMA, SLAMF7, CD38, FcFR5 or GPRC5D or the protein which is expressed in the surface of the cells of the immune system is CD3, CD28 or CD137.

122. The genetically engineered mouse according to claim 118 wherein the protein which confers sensitivity to an anti-cell blood cancer therapy is an immune checkpoint and the anti-cell blood cancer therapy is a therapy based on an immune checkpoint inhibitor.

123. The genetically engineered mouse according to claim 122 wherein the immune checkpoint is 4-1-BB1, PD-1 and PD-L1.

124. The genetically engineered mouse according to claim 95 suffering from human-like multiple myeloma (MM).

125. A method for the induction of multiple myeloma (MM) in a genetically engineered mouse according to claim 95 comprising maintaining the mouse under conditions adequate for the development of multiple myeloma.

126. A method for the screening for a candidate substance for the treatment of a MM, which method comprises the steps of:
(i) providing a genetically engineered mouse according to claim 96 and inducing in said mouse the proliferation of plasma cells until the presence of MM is detected or providing an mouse suffering from MM according to claim 113;
(ii) administering said candidate substance to said mouse;
(iii) determining the effect of the candidate substance on the MM,
wherein an increased effect of the candidate compound on the MM with respect to the effect observed in a mouse treated with a control substance indicates that the candidate compound is suitable for the treatment of MM.
